(19) United States
(12) Patent Application Publication
(10) Pub. No.: US 2006/0078929 A1
(43) Pub. Date: Apr. 13, 2006

(54) Device for the amplification and detection of nucleic acids

(75) Inventors: Ralf Bickel, Jena (DE); Thomas Ellinger, Jena (DE); Eugen Ermantraut, Jena (DE); Thomas Kaiser, Jena (DE); Torsten Schulz, Jena (DE); Thomas Ullrich, Jena (DE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Clondiag Chip Technologies GmbH, Jena (DE)

(21) Appl. No.: 11/241,671

(22) Filed: Sep. 30, 2005

Related U.S. Application Data
(63) Continuation of application No. PCT/EP04/03532, filed on Oct. 14, 2004.

(30) Foreign Application Priority Data
Apr. 2, 2003 (DE) 103 15 074.9

(51) Int. Cl. C40B 40/08; G06F 19/00; C12M 1/34

(52) U.S. Cl. 435/6; 435/287.2; 702/20

(57) ABSTRACT

The present invention relates to a device for the amplification and for the detection of nucleic acids comprising a temperature controlling and/or regulating unit; a reaction chamber containing a support with a detection area, on which a compound library is immobilized, wherein the temperature in the reaction chamber can be controlled and/or regulated by means of the temperature controlling and regulating unit; and an optical system, by means of which the time-dependent behavior of precipitate formations on the detection area is detectable. Methods of using the device are also disclosed.

### CROSS-REFERENCE TO RELATED APPLICATIONS

The present application is a continuation of International Application PCT/EP2004/003532, filed Oct. 14, 2004, published in German, and which claims priority from German Application No. 103 15 074.9, filed Apr. 2, 2003. The disclosures of which are incorporated herein by reference.

### BACKGROUND OF THE INVENTION

The invention relates to devices and methods for the amplification of nucleic acids and for the detection of specific interactions between molecular target and probe molecules.

Biomedical tests are often based on the detection of an interaction between a molecule, which is present in known amount and position (the molecular probe), and an unknown molecule to be detected or unknown molecules to be detected (the molecular target molecules). In modern tests, the probes are laid out in the form of a substance library on supports, the so-called microarrays or chips, so that a sample can be analyzed simultaneously at various probes in a parallel manner (see for example D. J. Lockhart, E. A. Winzeler, Genomics, gene expression and DNA arrays; Nature 2000, 405, 827-836). Herein, the probes are usually immobilized on a suitable matrix, as for example described in WO 00/12575 (see for example U.S. Pat. No. 5,412,087, WO 98/36827), or synthetically produced (see for example U.S. Pat. No. 5,143,854) in a predetermined manner for the preparation of the microarrays.

The detection of an interaction between the probe and the target molecule is usually conducted as follows: Subsequently to fixing the probe or the probes to a specific matrix in the form of a microarray in a predetermined manner, the targets are contacted with the probes in a solution and are incubated under defined conditions. As a result of the incubation, a specific interaction occurs between probe and target. The bond occurring herein is significantly more stable than the bond of target molecules to probes, which are not specific for the target molecule. In order to remove target molecules, which have not specifically been bound, the system is washed with corresponding solutions or is heated.

The detection of the specific interaction between a target and its probe can be performed by means of a variety of methods, which normally depend on the type of the marker, which has been inserted into target molecules before, during or after the interaction of the target molecule with the microarray. Typically, such markers are fluorescent groups, so that specific target/probe interactions can be read out fluorescence optically with high local resolution and, compared to other conventional detection methods, in particular mass-sensitive methods, with low effort (A. Marshall, J. Hodgson, DNA chips: An array of possibilities, Nature Biotechnology 1998, 16, 27-31; G. Ramsay, DNA Chips: State of the art, Nature Biotechnology 1998, 16, 40-44).

Depending on the substance library immobilized on the microarray and the chemical nature of the target molecules, interactions between nucleic acids and nucleic acids, between proteins and proteins, and between nucleic acids and proteins can be examined by means of this test principle (for survey see F. Lottspeich, H. Zorbas, 1998, Bioanalytik, Spektrum Akademischer Verlag, Heidelberg/Berlin, Germany).

Herein, antibody libraries, receptor libraries, peptide libraries, and nucleic acid libraries are considered as substance libraries, which can be immobilized on microarrays or chips.

The nucleic acid libraries play the most important role by far. These are microarrays, on which deoxyribonucleic acid (DNA) molecules or ribonucleic acid (RNA) molecules are immobilized.

It is a prerequisite for binding a target molecule, which is for example labeled with a fluorescence group, in the form of a DNA or RNA molecule to a nucleic acid of the microarray that both the target molecule and the probe molecule are present in the form of a single-stranded nucleic acid. An efficient and specific hybridization can only occur between such molecules. Single-stranded nucleic acid target and nucleic acid probe molecules are normally obtained by means of heat denaturation and optimal selection of parameters like temperature, ionic strength, and concentration of helix-destabilizing molecules. Therefore, it is warranted that only probes having sequences of almost perfect complementarity, i.e., closely corresponding to one another, remain paired with the target sequence (A. A. Leitch, T. Schwarzacher, D. Jackson, I. J. Leitch, 1994, In vitro Hybridisierung, Spektrum Akade-mischer Verlag, Heidelberg/Berlin/Oxford).

A typical example for the use of microarrays in biological test methods is the detection of microorganisms in samples in biomedical diagnostics. Herein, it is taken advantage of the fact that the genes for ribosomal RNA (rRNA) are spread ubiquitously and have sequence portions, which are characteristic for the respective species. Said species-characteristic sequences are laid out on a microarray in the form of single-stranded DNA oligonucleotides. The target DNA molecules to be examined are first isolated from the sample to be examined and are attached to markers, for example fluorescence markers. Subsequently, the labeled target DNA molecules are incubated in a solution together with the probes laid out on the microarray; unspecifically occurring interactions are removed by means of corresponding washing steps and specific interactions are detected by means of fluorescence optical evaluation. In this manner, it is possible to detect, for example, various microorganisms in a sample simultaneously by means of one single test. In this test method, the number of detectable microorganisms theoretically depends only on the number of specific probes, which have been laid out on the microarray.

A variety of methods and technical systems, some of which are also commercially available, are described for the detection of molecular interactions with the aid of microarrays or probe arrays on solid surfaces.

Classical systems for the detection of molecular interactions are based on the comparison of the fluorescence intensities of spectrally excited target molecules labeled with fluorophores. Fluorescence is the capacity of particular molecules to emit their own light when excited by light of a particular wavelength. In this case, a characteristic absorption and emission behavior ensues. In analysis, a proportional increase of the fluorescence signal is assumed as labeled molecule density on the functionalized surface increases, for example due to increasing efficiency of the molecular interaction between target and probe molecules.

In particular, quantitative detection of fluorescence signals is performed by means of modified methods of fluorescence microscopy. Herein, the light having the absorption wavelength is separated from the light having the emission wavelength by means of filters or dichroites and the measurement signal is imaged on suitable detectors, like for example two-dimensional CCD arrays, by means of optical elements like objectives and lenses. In general, analysis is performed by means of digital image processing.

The technical solutions known hitherto vary regarding their optical setup and the components used. Problems and limitations of such setups result from the signal noise (the background), which is essentially determined by effects like bleaching and quenching of the colorants used, autofluorescence of the media, assembling elements, and optical components as well as by dispersions, reflections, and secondary light sources within the optical setup.

Resulting therefrom is a high technical effort for the setup of highly sensitive fluorescence detectors for the qualitative and quantitative comparison of probe arrays. In particular, for screening with medium and high throughputs, specially adapted detection systems are necessary, which exhibit a certain degree of automation.

For optimizing standard epifluorescence setups for reading out molecular arrays, CCD-based detectors are known, which implement the excitation of the fluorophores in the dark field by means of incident light or transmitted light for the discrimination of optical effects like dispersion and reflections (see for example C. E. Hooper et al., Quantitative Photon Imaging in the Life Sciences Using Intensified CCD Cameras, Journal of Bioluminescence and Chemiluminescence (1990), S. 337-344). Herein, the imaging of the arrays is performed either in an exposure or by means of rasterizing using higher resolution optics. The use of multispectral light sources allows a comparatively easy access to different fluorophores by means of using different excitation filters (combinations). However, it is a disadvantage that autofluorescence and system-related optical effects like the illumination homogeneity above the array necessitate complicated illumination optics and filter systems.

Further methods for the quantitative detection of fluorescence signals are based on the confocal fluorescence microscopy. In a typically confocal layout, the object in the focal plane of the objective is illuminated by a point light source. The light reflected by the object is mirrored in the direction of an optical point light detector by means of a beam splitter and is detected. Herein, point light source, object and point light detector are located on exactly optically conjugated planes. Therefore, light coming from outside the focal plane is not focused sharply at the detector and for this reason is not even recorded. Thus, object parts being located above or below the focal plane are blanked. The advantage of the confocal method lies in this elimination of the disturbing portion of dispersed light outside the focal plane. The total image is generated by means of directing the light point across the object in lines. This is also referred to as scanning. The image data records recorded in this rasterizing procedure are subsequently combined to form a 2D or 3D image.

It is the task of the scanning unit to direct the light beam in a rasterizing manner across the object located under the microscope. To this end, there are principally four possibilities:

a) The object is directed past the stationary laser on a movable table. Herein, the laser is in inoperative position.

b) The object is movable and is directed past the stationary laser. Herein also, the laser is in inoperative position.

c) The movable laser beam is directed past the object. Herein, the object is in inoperative position.

d) The laser beam is moved in one axis, the object is moved in the other axis.

Confocal scanning systems, as for example described in U.S. Pat. No. 5,304,810, are based on the selection of the fluorescence signals along the optical axis by means of two pinholes. This results in a high adjustment complexity for the samples and the establishment of an effective autofocus system, respectively. Such systems are highly complex regarding their technical implementation. Necessary components like lasers, pinholes, optionally cooled detectors, like for example PMT, avalanche diodes or CCD, complex and highly exact mechanical translation elements and optical systems have to be optimally coordinated demanding considerable effort (see for example U.S. Pat. No. 5,459,325; U.S. Pat. No. 5,192,980; U.S. Pat. No. 5,834,758). The degree of miniaturization and the price are limited by the variety and functionality of the components.

At this point in time, analyses based on probe arrays are generally read out fluorescence optically (see for example A. Marshall und J. Hodgson, DNA Chips: An array of possibilities, Nature Biotechnology, 16, 1998, 27-31; G. Ramsay, DNA Chips: State of the Art, Nature Biotechnology, 16, Jan. 1998, 40-44). However, the disadvantages of the above-described detection devices and methods are the high-level signal background, which leads to limited exactitude, the partially considerable technical effort, as well as the high costs, which are associated with the detection methods.

A variety of, in particular, confocal systems are known, which are suitable for the detection of lowly integrated substance libraries in array format, which are installed in fluidic chambers (see for example U.S. Pat. No. 5,324,633, U.S. Pat. No. 6,027,880, U.S. Pat. No. 5,585,639, WO 00/12759).

The above-described methods and systems can only be adapted for the detection of highly integrated molecular arrays, which are, in particular, installed in fluidic systems, in a very limited way, in particular due to the dispersions, reflections, and optical aberrations occurring therein. Furthermore, in such highly integrated arrays, great demands are made concerning the spatial resolution, which could up to now not be implemented technically, however.

Thus, there is a need for highly integrated arrays, wherein the interaction between probes and targets can be detected qualitatively and/or quantitatively with great exactitude and at comparatively low technical effort.

The increase of selectivity and the access to alternative components motivate the establishment of alternative imaging technologies like fluorescence polarization and time-resolved fluorescence for assays bound to solid bodies. Such solutions, in particular for highly integrated arrays, only exist in the form of concepts up to now, however. The effect of twisting the polarization axis by means of fluorophores excited in a polarized manner is used for quantification in the microwell format. Furthermore, there are approaches to set up inexpensive systems having a high throughput (HTS systems) by means of using correspondingly modified polymer foils as polarization filters (see I. Gryczcynski et al., Polarisation sensing with visual detection, Anal. Chem. 1999, 71, 1241-1251). However, the presently available light quantities and detectors makes an implementation for microarrays difficult. Such a setup would necessitate the integration of light sources (for example lasers, LED, high pressure lamps), polarization filters (possibly coated polymer foils), and detectors (CCD, CMOS camera); a corresponding solution is hitherto unknown.

More recent developments utilize the fluorescence of inorganic materials, like lanthanides (M. Kwiatowski et al., Solid-phase synthesis of chelate-labelled oligonucleotides: application in triple-color ligase-mediated gene analysis, Nucleic Acids Research, 1994, 22, 13) and quantum dots (M. P. Bruchez et. al., Semiconductor nanocrystals as fluorescent biological labels, Science 1998, 281, 2013). The utilization of the specific fluorescence lifetime of fluorophores in the range of nanoseconds for their selective quantification is very complex and is not used commercially despite the specificity in this locally resolved application. Colorants exhibiting long emission time within a range of microseconds, like lanthanide chelates, necessitate a conversion of the colorants to a mobile phase, so that a locally resolved detection is not possible.

The use of microparticles, which are known from their use in television tubes (see F. van de Rijke et al., Up-Converting Phosphors: A New Reporter Technology for Nucleic Acid Microarrays, European EC Meeting on Cytogenetics (2000) Bari, Italy), as biological markers is of great potential concerning sensitivity and miniaturizability of the setup of the detection technics, especially since light sources from the field of data transmission are used for the excitation (980 nm diode laser). However, for the detection of target/probe interactions on arrays, this technology is commercially not available at present. A detector would comprise components for light emission (for example laser, LED, high pressure lamps), a system for modulating the excitation and detection light (for example chopper discs, electronic shutters) and detecting the time-delayed signal (for example CCD, CMOS camera). In general, the low compatibility of the particles with biological samples is a basic problem.

Optical setups for the detection of samples labeled by means of gold beads and their visualization by means of silver amplification are described in WO 00/72018. The devices described therein are only suitable for detection in static measurement, however. In static measurement, subsequently to the interaction of the targets with the probes laid out on the probe array and subsequently to the beginning of the reaction leading to precipitation on those array elements, where an interaction has occurred, an image is recorded and assigned to the measured concentrations of gray values, which depend on the degree of precipitation formation.

It was described in the International Patent Application WO 02/02180 that this static measurement procedure leads to satisfactory values only within a very narrow range of concentrations and is therefore problematic also for the evaluation of the specificity of interactions, as the precipitation formation does largely not occur in a linear manner. In particular, the time-dependent behavior of the precipitation formation comprises an exponential increase of precipitation formation over time as well as a subsequent saturation level. Only gray values from in the range of the exponential increase of the precipitation formation over time allow a correlation with the amount of targets bound, while the saturation level, which is reached with precipitation formation on an array element after a certain time, which is depending on the respective probe target interaction and is therefore different for each array element, is opposed to a quantification after completion of the precipitation formation reaction. It is not possible to design the experiment parameters in such a way that it can be ensured without any doubt that the saturation level is reached on none of the array elements, because the reaction speed largely depends on temperature, light, salt concentration, pH, and other factors.

In the case of recording only one image, i.e., in static measurement, it can therefore not be ensured that the precipitation formation reactions on all array elements are within the exponential range of the dependency of precipitation formation on time. Therefore, signal intensities, for example gray values, which are obtained from those array elements, where the precipitation reaction already is at saturation level, in comparison with signal intensities of array elements, which are still within the exponential range of the precipitation formation, are represented in a falsified manner.

In order to overcome the above-described disadvantages, a method for the qualitative and/or quantitative detection of targets in a sample by means of molecular interactions between probes and targets on probe arrays was provided in WO 02/02810, wherein the time-dependent behavior of precipitation formation at the array elements is detected in the form of signal intensities, i.e., dynamic measurement is performed. On the basis of a curve function describing the precipitation formation as a function of time, a value quantifying the interaction between probe and target on an array element and therefore the amount of targets bound is assigned to each array element.

Such dynamic measurement requires the recording of image series under, for example, particular thermal conditions or in a particular phase of a procedure, for example in the presence of particular solutions at the time of the recording. This requires a complex cooperation of the individual components of a highly integrated array, in particular in the case of uses in the field of genotyping.

Furthermore, in many tests in biomedical diagnostics, there arises the problem that the target molecules are at first not present in an amount sufficient for a detection and therefore first have to be amplified from the sample before the actual test procedure. Typically, the amplification of DNA molecules is performed by means of the polymerase chain reaction (PCR). For the amplification of RNA, the RNA molecules have to be converted to respectively complementary DNA (cDNA) by means of reverse transcription. Said cDNA can then also be amplified by means of PCR. PCR is a standard laboratory method (like for example in Sambrook et al. (2001) Molecular Cloning: A laboratory manual, 3rd edition, Cold Spring Harbor, N.Y., Cold Spring Harbor Laboratory Press).

The amplification of DNA by means of PCR is comparatively fast, allows a high sample throughput in small setup volumes by means of miniaturized methods, and is efficient in operation by means of automation.

However, a characterization of nucleic acids by means of mere amplification is not possible. It is rather necessary to use analysis methods like nucleic acid sequence determinations, hybridization, and/or electrophoretic separation and isolation methods for the characterization of the PCR products subsequently to the amplification.

In general, devices and methods for the amplification of nucleic acids and their detection should be designed in such a way that as few interventions by the experimenter as possible are necessary. The advantages of methods allowing a amplification of nucleic acids and their detection, and in the course of which the experimenter has to intervene only minimally, are obvious. On the one hand, contaminations are avoided. On the other hand, the reproducibility of such methods is substantially increased, as they are accessible to automation. This is also extremely important considering the approval of diagnostic methods.

At present, there are a multiplicity of methods for the amplification of nucleic acids and their detection, in which first the target material is amplified by means of PCR amplification and subsequently the identity or the genetic state of the target sequences is determined by means of hybridization against a probe array. In general, the amplification of the nucleic acid molecules and/or the target molecules to be detected is necessary in order to have at one's disposal amounts sufficient for a qualitative and quantitative detection within the scope of the hybridization.

Both the PCR amplification of nucleic acids and their detection by means of hybridization are subject to several elementary problems. In the same manner, this applies to methods combining a PCR amplification of nucleic acids and their detection by means of hybridization.

One of the problems arising in methods combining PCR and hybridization is based on the double-strandedness of the target molecules. Assuming a double-stranded template molecule, classical PCR amplification reactions usually produce double-stranded DNA molecules. These can only hybridize with the probes of the probe array after previous denaturation. During the hybridization reaction, the very rapid formation of double strands in the solution with the hybridization competes against the immobilized probes of the probe array. The intensity of the hybridization signals, and therefore the quantitative and qualitative evaluation of the results of the method, is strongly limited by this competition reaction.

In addition, there are the problems based on the hybridization reaction per se and on the probes and targets made to hybridize, respectively. PCR products used as targets for array hybridization reactions normally have a length of at least about 60 base pairs. This corresponds to the sum of the lengths of the forward and reverse primers used for the PCR reaction as well as to the region, which is amplified by the PCR and which exhibits complementarity to the probe on the array. Single-stranded molecules of this length are seldom present in solution in an unstructured form, i.e., linearly stretched, but have more or less stable secondary structures like e.g., hairpins or other helical structures. If these secondary structures affect the target region, which exhibits complementarity to the probe, the formation of said secondary structures prevents an efficient hybridization of the target to the probe. Therefore, the formation of secondary structures can also inhibit an efficient hybridization and complicate, if not prevent, a quantitative and qualitative evaluation of the results of the method.

Thus, there is furthermore a need for devices, which allow the performance of PCR and analysis reaction, like for example a hybridization reaction, in one reaction chamber.

If detectable markers, for example in the form of fluorescence labeled primers, are inserted into the nucleic acid molecules to be detected or target molecules to be detected in a method, which combines a PCR amplification and its detection by means of hybridization, a washing step is usually performed before the actual detection. Such a washing step serves the removal of the unconverted primers, which are present in great abundance compared to the amplification product, as well as of such nucleotides equipped with a fluorescence marker, which do not participate in the detection reaction and/or do not specifically hybridize with the nucleic acid probes of the microarray. In this manner, the high-level signal background caused by these molecules shall be decreased. However, such an additional procedure step considerably slows down the detection method. Furthermore, the detectable signal is considerably decreased also for those nucleic acids to be detected, which specifically hybridize with the nucleic acid probes of the microarray. The latter is largely based on the fact that the equilibrium between those targets bound by means of hybridization and those situated in solution does not exist anymore after the washing step. Nucleic acids, which had already hybridized with the nucleic acids on the array, are displaced from the binding site by the washing and are therefore washed away together with the molecules in the solution. Altogether, there only remains a detectable signal, if the washing or rinsing step of the molecules in solution is performed faster than the displacement of the nucleic acids already hybridized.

Therefore, there is a need for highly integrated arrays, wherein the interaction between probes and targets can be detected qualitatively and/or quantitatively with great accuracy and with comparatively low technical effort.

Furthermore, there is a need for devices, which allow the performance of PCR and analysis reaction, like for example a hybridization reaction, in one reaction chamber.

Furthermore, there is a need for highly integrated arrays, wherein the quantitative interaction between probes and targets can be detected with great accuracy by means of detecting the time-dependent behavior of a precipitation formation, like for example by means of detecting samples labeled with gold beads and their visualization by means of silver amplification.

It is therefore a problem underlying the present invention to overcome the above-mentioned problems of the art, which in particular arise due to the lack of compatibility of the assay with the test system.

In particular, it is a problem underlying the present invention to provide a method and/or a device, wherein molecular interactions between probes and targets on probe arrays can be qualitatively and/or quantitatively detected with great accuracy and high sensitivity as well as in an easy-to-do and cost-efficient manner.

Furthermore, it is a problem underlying the present invention to provide methods and/or devices for the amplification and for the qualitative and quantitative detection of nucleic acids, wherein interventions by the experimenter can be minimized.

It is a further problem underlying the present invention to provide methods and devices for the amplification and for the qualitative and quantitative detection of nucleic acids, wherein a high signal-to-noise ratio in the detection of interactions on the microarray is ensured without impairing the hybridization between the nucleic acids to be detected and the nucleic acid probes on the array.

It is a further problem underlying the present invention to provide a device, by means of which a high dynamic resolution in detection is achieved, i.e., the detection of weak probe target interactions among strong signals remains is ensured.

Furthermore, it is a problem underlying the present invention to provide a device, which allows an almost simultaneous amplification and characterization of nucleic acids at a high throughput rate.

### SUMMARY OF THE INVENTION

These and further problems underlying the present invention are solved by means of providing the embodiments characterized in the patent claims.

In particular, a method for the amplification and for the qualitative and quantitative detection of nucleic acids in a sample is provided within the scope of the present invention, comprising the following steps:

a) inserting the sample into a reaction chamber, which is formed between a chamber support and a microarray, wherein the microarray comprises a substrate with nucleic acid probes immobilized on array elements thereon;

b) amplifying the nucleic acids to be detected in the reaction chamber by means of a cyclic amplification reaction;

c) detecting a hybridization between the nucleic acids to be detected and the nucleic acid probes immobilized on the substrate without removing those molecules from the reaction chamber, which are not hybridized with the nucleic acids immobilized on the substrate.

The detection can preferably be performed during the cyclic amplification reaction, i.e., during the course of one or more cycles of the amplification reaction, and/or after completion of the cyclic amplification reaction.

The method according to the present invention for the amplification of nucleic acids and their detection is designed in such a way, that as few interventions by the experimenter as possible are required. This offers the essential advantage that contaminations are thereby avoided. Furthermore, the reproducibility of the method according to the present invention is essentially increased compared to conventional methods, as the method according to the present invention is accessible to automation due to the minimization of external interventions. The above-mentioned advantages play an important role in terms of the approval of diagnostic methods.

Furthermore, according to the present invention, a device for the amplification and for the qualitative and quantitative detection of nucleic acids by means of a method as described above is provided comprising the following elements:

a) a temperature controlling and/or regulating unit;

b) a reaction chamber formed between a chamber support and a microarray, wherein the microarray comprises a substrate with nucleic acid probes immobilized on array elements thereon and wherein the temperature in the reaction chamber can be controlled and/or regulated by means of the temperature controlling and regulating unit;

wherein a hybridization between the nucleic acids to be detected and the nucleic acid probes immobilized on the substrate can be detected by means of the device without removing those molecules from the reaction chamber, which are not hybridized with the nucleic acids immobilized on the substrate.

Preferably, the reaction chamber of the device according to the present invention is developed as a capillary gap between the chamber support and the microarray.

In another aspect of the present invention, the problem is solved, according to the present invention, by providing a device for the amplification and detection of nucleic acids comprising at least one temperature controlling and/or regulating unit, a reaction chamber containing a detection area with a probe substance library immobilized thereon, as well as preferably an optical system, by means of which the time-dependent behavior of precipitation formations on the detection area can be detected.

A chip inside the reaction chamber, wherein the chip comprises a support with a detection area, whereon a substance library is immobilized, ensures the possibility of providing a very high probe density in the reaction chamber.

The electrocaloric control and/or regulation by means of the temperature controlling and/or regulating unit allows the setting of defined temperatures both during processing of the sample to be examined in the reaction chamber and during the detection of the hybridization events. Thus, both an improved control and an optimization of the detection reaction are ensured. Furthermore, the setting of defined temperatures by means of the temperature controlling and/or regulating unit allows the performance of complex reactions, like for example of amplification reactions by means of PCR.

The devices according to the present invention are further characterized in that it is possible to detect molecular interactions also in manual operation due to, inter alia, the preferably integrated optical system and/or the reader system. This is particularly advantageous in fields like that of medical diagnostics.

Due to the fact that the device according to the present invention in one aspect of the present invention preferably contains an integrated optical system, by means of which the time-dependent behavior of precipitation formations on the detection area is detectable, an exact detection of the relative quantitative amounts of nucleic acids bound to the substance libraries is ensured.

In general, the devices according to the present invention allow a performance of processing and/or conditioning reactions, which is almost simultaneous, time-efficient and exhibits a low fault liability as well as the chip-based characterization of nucleic acids. Herein, according to the present invention, a processing and/or conditioning reaction is understood to denote a reaction, whose reaction products can be characterized by means of chip-based experiments.

A device according to the present invention for the detection of molecular interactions in closed reaction chambers preferably consists of four principal functional elements (see FIG. 1). The mechanical, electrical, and fluidic recording of the reaction chamber is performed in a recording module (1). In the following, the reaction chamber is also referred to as microreactor. For the detection of the reaction results, an optical system (2) is provided. The processing of the reaction results to an analysis result can be performed in a controller (3). Optionally, the analysis result is made available for storage and/or further processing by means of suitable connecting elements (4).

A reaction chamber, which can be used as component of the device according to the present invention in an advantageous manner, is described in detail in the International Patent Application WO 01/02094, whose contents are hereby explicitly referred to.

The reaction chamber, which can optionally be identified by means of a bar code, is integrated in a fluidic recording module, where it can be filled with one or more reaction solutions. Optionally, the reaction chamber further has electrical contacts, whereby a thermal control and/or regulation of reactions in the reaction chamber, for example by means of integrated sensor and/or heating elements, is ensured. In particular, this is advantageous for the performance of thermally sensitive amplification reactions for DNA or RNA, hybridizations of DNA or RNA, or reactions for the enhancement of signals, like for example by means of metal precipitations at target molecules, which are correspondingly labeled and bound to the substance library.

The solutions optionally required for the performance of the amplification and detection reactions, like reaction and/or rinsing solutions, can be inserted into the reaction chamber via suitable connecting elements, like for example channels. Suitable controllers can be used for the supervision of the course of the reaction.

In one aspect of the present invention, the optical system ensures the imaging of the substance library during or after completion of the amplification and/or detection reactions on a suitable detector, which is for example implemented in the form of a two-dimensional, electrically readable detection element. In one embodiment of the device according to the present invention, the sample is illuminated by means of an illumination module or a light source of the optical system and the emerging signals are imaged in a filtered manner, according to the labels used.

In one aspect of the present invention, the optical system further ensures a kinetic, i.e., dynamic, recording of the reaction events. In particular, the optical system of the device according to the present invention is preferably suitable for recording the time-dependent behavior of a silver precipitation for enhancing the hybridization signals between gold-labeled target molecules and the substance library. The highly integrated setup of the device according to the present invention allows the transfer of several images during the course of the reaction for processing in a suitable data processing module or controller.

The alterations caused by the time-dependent behavior of the precipitation formation on the respective array elements can be evaluated in the device according to the present invention, as is described in detail, inter alia, in the International Patent Application WO 02/02810. The contents of the International Patent Application WO 02/02810 are hereby also explicitly referred to.

The device according to the present invention further ensures the transfer of the raw data or analysis results to external computers or computer networks, for example for storage of said data, via optionally existing electronic interfaces.

In another aspect of the present invention, a microarray comprising a substrate, whereon molecular probes are immobilized on predetermined regions, is provided. Herein, the substrate essentially comprises ceramic materials.

Finally, a further aspect relates to the use of a substrate essentially made of ceramic materials for manufacturing a microarray having molecular probes immobilized on predetermined regions of the substrate thereon.

### BRIEF DESCRIPTION OF THE DRAWINGS

For the purposes of illustrating the invention, there are shown in the drawings illustrative embodiments of various aspects of the invention. It is understood that these drawings depict only selected embodiments of the invention and are therefore not to be considered limiting of its scope.

FIG. 1: Schematic representation of a processing and detection device according to the present invention for the amplification and detection of nucleic acids.

FIG. 2: Schematic representation of a processing and detection device according to the present invention for the amplification and detection of nucleic acids having an external process controller and, depending on individualization of the chip, variably selectable detection systems.

FIG. 3: Schematic representation of a processing and detection device according to the present invention for the amplification and detection of nucleic acids without fluid processing unit.

FIG. 4: Incident-light arrangement of the device according to the present invention.

FIG. 5: Dark-field arrangement of the device according to the present invention.

FIG. 6: Representation of an image sequence for documenting the epitaxial growth of silver particles in the hybridization of gold-labeled target molecules with probe molecules immobilized on the detection area in the form of a substance library.

FIG. 7: Schematic representation of a processing and detection device according to the present invention for the amplification and detection of nucleic acids by means of electric detection of the kinetics of the silver precipitation at one individual planar array spot covered with probe molecules.

FIG. 8: Schematic representation of a processing and detection device according to the present invention for the amplification and detection of nucleic acids by means of electric detection of the kinetics of the silver precipitation at one individual array spot covered with probe molecules and having through holes.

FIG. 9: Process of the exponential amplification of a target with different initial concentrations of nucleic acids to be detected in the sample.

What is represented is the dependency of the concentration of the hybridized target nucleic acids on the number of cycles of the amplification reaction for different initial concentrations.

FIG. 10: Development of a hybridization signal with the use of an 8-bit detector in dependency on the number of amplification cycles and on the initial concentration of the target nucleic acids in solution as a result of the exponential amplification (see Example 4).

FIG. 11: Representation of the correlation between chamber thickness and the number of molecules labeled with a fluorescence marker, which are located in the supernatant immediately above the spot. The lines represent different concentrations of target molecules in solution (in pM), calculated for a volume of 10,000 μm² (corresponding to the area of a spot or array element), multiplied by the thickness of the chamber.

FIG. 12: Characteristic melting curve for different probes and specific course of duplex dissociation in dependency on temperature and the respective sequence (see Example 3).

FIG. 13: Schematic representation of the layout of probes on the array used in Example 5. Herein, one box represents four redundant probes.

FIG. 14: Probe array on a ceramic surface after hybridization and detection by means of enzymatic precipitation of an organic molecule (see Example 5). The layout of the array is illustrated in FIG. 13.

FIG. 15: Results of the hybridization of an oligonucleotide mixture against a probe array on a ceramic surface with subsequent detection by means of enzymatic precipitation of an organic molecule (see Example 5).

FIG. 16: Representation of an embodiment of the ceramic substrate according to the present invention for defined temperature direction in a device according to the present invention, a so-called assay processor.

The ceramic substrate and the assay processor have the following specification:

Dimensions: 12.0 mm×12.4 mm×0.635 mm

Support material: ceramic support

Sensor/heater structures: pt thin film structures

Passivation: glass-passivated

Connection: Pt paste, screen printed

Sensor: Pt 100, KI. A; TK=3850 ppm/K

Heater: 125 Ohm±8 Ohm

Likewise, sensors of the types Pt 1000, Pt 10 000 and so on are conceivable.

FIG. 17: Schematic representation of the assay processor shown in FIG. 16.

FIGS. 18 to 24: Schematic representations of preferred embodiments of the devices according to the present invention.

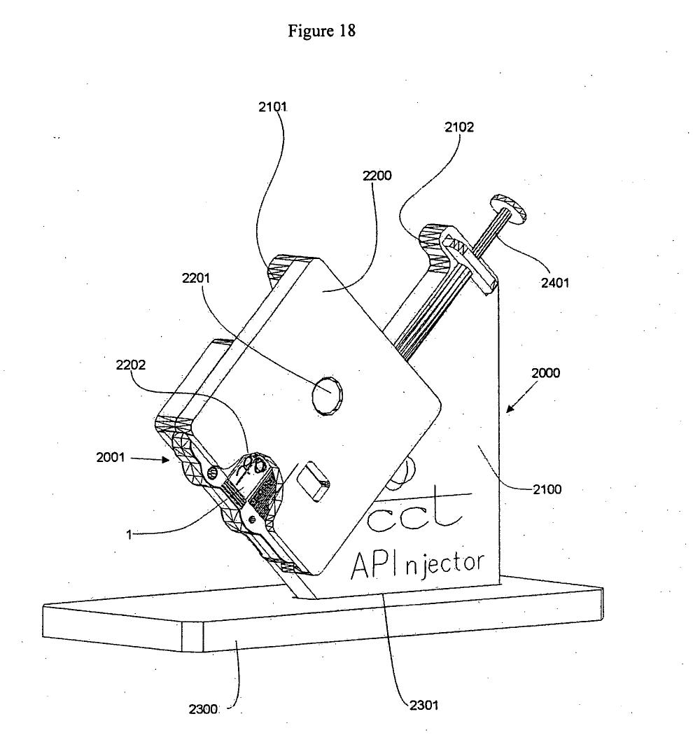

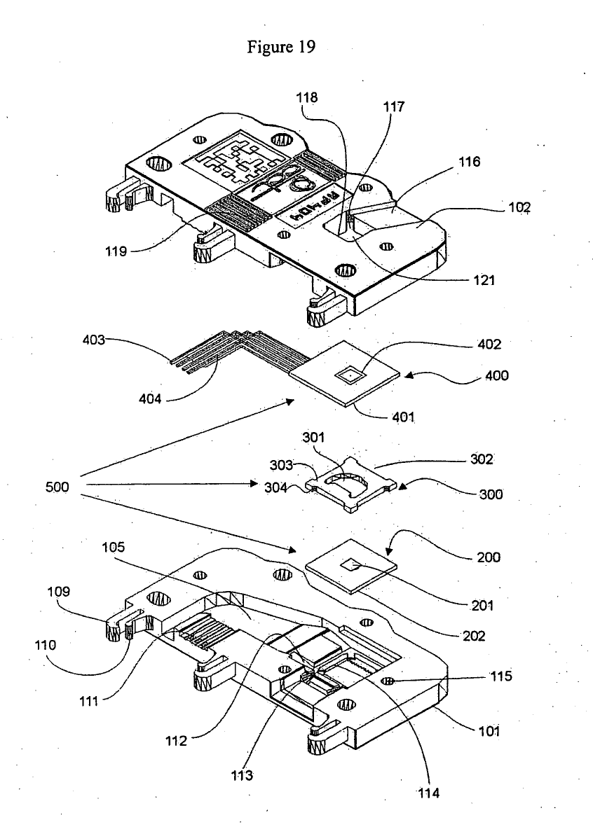

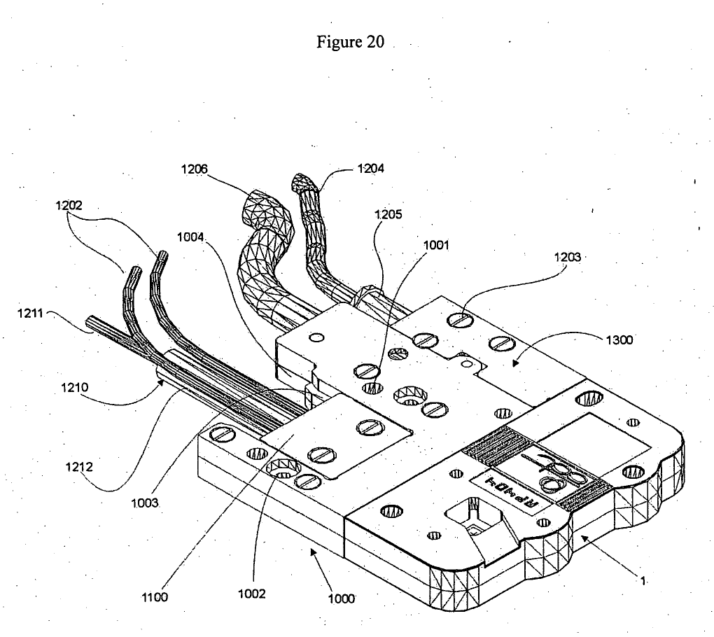

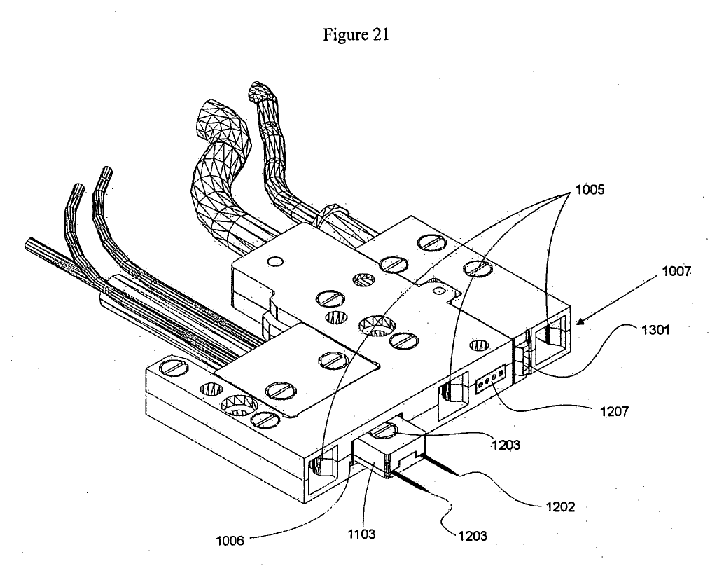

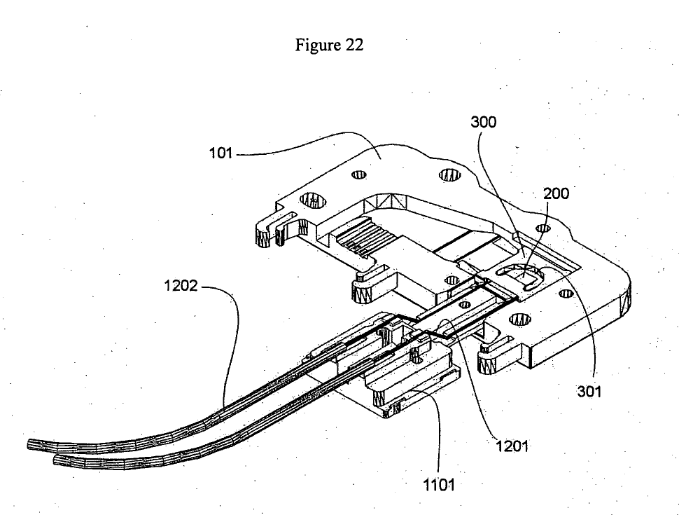

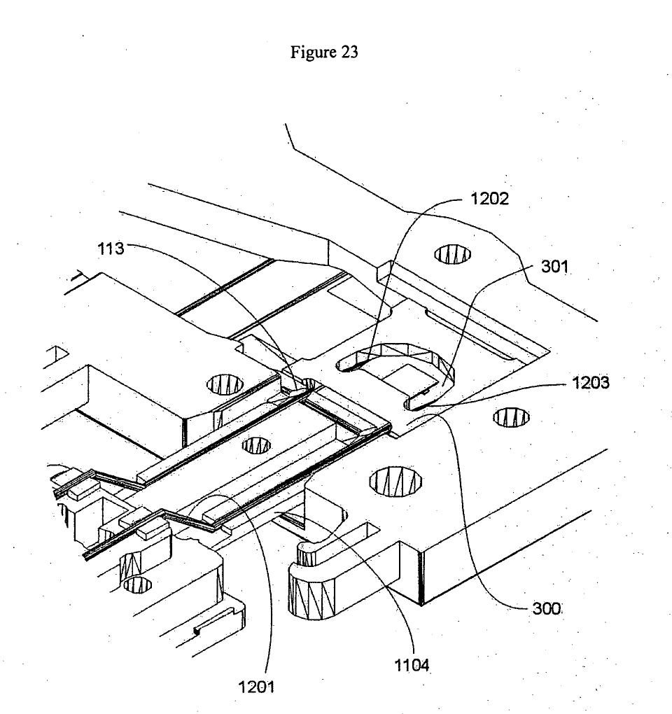

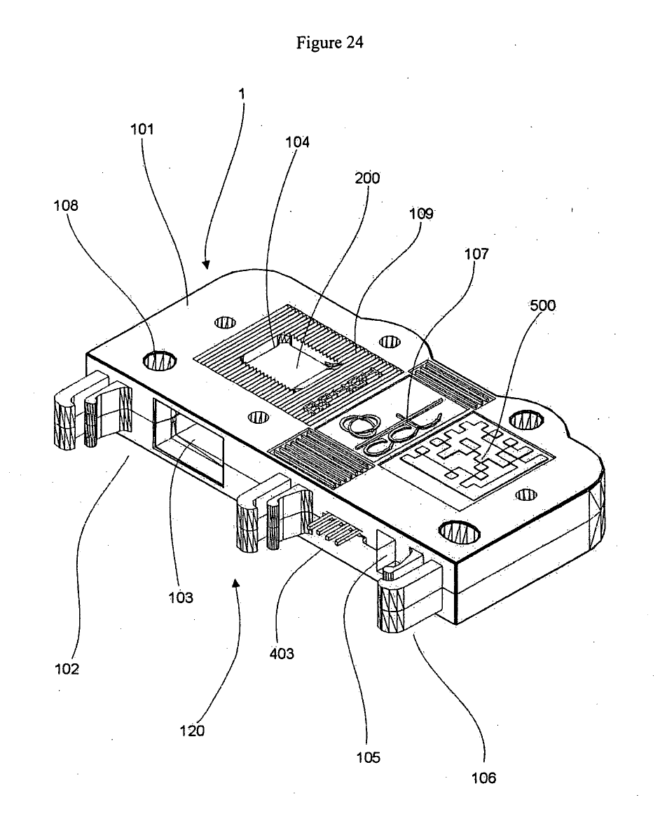

In the following, the list of reference numbers for FIGS. 18 to 24 is given:

1 cartridge

101 upper support element

102 lower support element

103 recess for retaining a catch/slide

104 window

105 medium connection for coolants

106 snap-fit

108 dowel pin holes

403 contacts for heating element

120 media connection side

109 anti-reflection structure

600 data matrix

117 cooling duct outlet

118 counter bearing

400 ground element with integrated heater/sensor substrate

401 support of the integrated heater/sensor substrate

402 optically transparent recess

403 contacts for heater/sensor substrate

404 contacts for heater/sensor substrate

300 sealing, elastic, repeatably puncturable intermediate element

301 enclosed recess in 300, defines reaction chamber

302 expansion bands

304 adjusting lugs

200 cover element

202 covering support element

201 substance library or chip bearing substance library

500 core assembly or chamber or reaction chamber or sample chamber

105 cooling duct

109 snap-fit

110 snap-fit

111 recess for heating/sensor substrate contacts (403)

112 coolant entrance

116 coolant outlet

117 coolant entrance

115 press fit

1000 connector

1100 slide

1103 guiding catch

1001 dowel pin holes

1002 mounting holes

1206 electric cable

1207 electric supply

1202 filling hoses

1210 radius rod

1211 screw thread

1212 damping

1204 coolant hose

1205 coolant connection

1300 cooling duct

1201 plunging hollow needles

2000 manual filling station

2106 quick-release connector

2401 disposable syringe

2404 hollow needle

2403 venting hollow needle

2402 tip of a hollow needle

2403 tip of a hollow needle

2100 basic body of manual filling station

2200 cover of manual filling station

2101 recessed grip

2102 recessed grip

### DETAILED DESCRIPTION

The following definitions, inter alia, are used for the description of the present invention:

Within the scope of the present invention, a probe, a probe molecule, or a molecular probe is understood to denote a molecule, which is used for the detection of other molecules due to a particular characteristic binding behavior and/or a particular reactivity. Each type of molecules, which can be coupled to solid surfaces and have a specific affinity, can be used as probes laid out on the array. In a preferred embodiment, these are biopolymers from the classes of peptides, proteins, nucleic acids, and/or analogs thereof. Particularly preferably, the probes are nucleic acids and/or nucleic acid analogs.

In particular, nucleic acid molecules of defined and known sequence, which are used for the detection of target molecules in hybridization methods, are referred to as probe. Both DNA and RNA molecules can be used as nucleic acids. For example, the nucleic acid probes or oligonucleotide probes can be oligonucleotides having a length of 10 to 100 bases, preferably of 15 to 50 bases, and particularly preferably of 20 to 30 bases. Typically, according to the present invention, the probes are single-stranded nucleic acid molecules or molecules of nucleic acid analogs, preferably single-stranded DNA molecules or RNA molecules having at least one sequence region, which is complementary to a sequence region of the target molecules. Depending on detection method and use, the probes can be immobilized on a solid support substrate, for example in the form of a microarray. Furthermore, depending on the detection method, they can be labeled radioactively or non-radioactively, so that they are detectable by means of the detection reaction conventional in the state of the art.

Within the scope of the present invention, a target or a target molecule is understood to denote a molecule to be detected by means of a molecular probe. In a preferred embodiment of the present invention, the targets to be detected are nucleic acids. However, the probe array according to the present invention can also be used for the detection of protein/probe interactions, antibody/probe interactions etc. in an analogous manner.

If, within the scope of the present invention, the targets are nucleic acids or nucleic acid molecules, which are detected by means of a hybridization against probes laid out on a probe array, said target molecules normally comprise sequences of a length of 40 to 10,000 bases, preferably of 60 to 2,000 bases, also preferably of 60 to 1,000 bases, particularly preferably of 60 to 500 bases and most preferably of 60 to 150 bases. Optionally, their sequence comprises the sequences of primers as well as the sequence regions of the template, which are defined by the primers. In particular, the target molecules can be single-stranded or double-stranded nucleic acid molecules, one or both strands of which are labeled radioactively or non-radioactively, so that they are detectable by means of a detection method conventional in the state of the art.

According to the present invention, a target sequence denotes the sequence region of the target, which is detected by means of hybridization with the probe. According to the present invention, said situation is also referred to as that region being addressed by the probe.

Within the scope of the present invention, a substance library is understood to denote a multiplicity of different probe molecules, preferably at least two to 1,000,000 different molecules, particularly preferably at least 10 to 10,000 different molecules and most preferably between 100 to 1,000 different molecules. In the case of special designs, a substance library can also comprise only at least 50 or less or at least 30,000 different molecules. Preferably, the substance library is laid out in the form of an array on a support inside the reaction chamber of the device according to the present invention.

Within the scope of the present invention, a probe array is understood to denote a layout of molecular probes or a substance library on a support, wherein the position of each probe is defined separately. Preferably, the array comprises defined sites and/or predetermined regions, so-called array elements, which are particularly preferably laid out in a particular pattern, wherein each array element usually comprises only one species of probes. Herein, the layout of the molecules or probes on the support can be generated by means of covalent or non-covalent interactions. A position within the layout, i.e., within the array, is usually referred to as spot. The probe array therefore forms the detection area.

Within the scope of the present invention, an array element, or a predetermined region, or a spot, or an array spot is understood to denote a particular area, which is determined for the deposition of a molecular probe, on a surface; the entirety of all occupied array elements is the probe array.

Within the scope of the present invention, a support element, or support, or substance library support, or substrate is understood to denote a solid body, on which the probe array is set up. The support, usually also referred to as substrate or matrix, can for example denote an object support or a wafer or ceramic materials.

The entirety of molecules laid out in array layout on the substrate or on the detection area, or of the substance library laid out in array layout on the substrate or on the detection area, and of the support or substrate is also often referred to as “chip”, “microarray”, “DNA chip”, “probe array” etc.

Within the scope of the present invention, a detection area is understood to denote the region of the substrate, where the substance library is laid out, preferably in array layout.

Within the scope of the present invention, a chamber support is understood to denote a solid body forming a base for the reaction chamber. Preferably, the chamber support is arranged on the side opposite of the substrate or of the substance library support. In an alternative embodiment of the device according to the present invention, the chamber support can also simultaneously be the substrate and/or the substance library support.

Within the scope of the present invention, a chamber body is understood to denote the solid body forming the reaction chamber. Usually, the substance library support or the chip is part of the chamber body, wherein the substance library support may consist of a different material than the remaining chamber body.

Within the scope of the present invention, a reaction chamber or reaction space is understood to denote the space formed between chamber support and microarray and preferably designed in the form of a capillary gap. The base of the reaction chamber or the reaction space is defined by the base of the array or of the chamber support, respectively. In particular, the distance between chamber body and matrix or microarray is referred to as thickness of the reaction space or reaction chamber. Within the scope of the present invention, a reaction space usually has only a small thickness, for example a thickness of at most 1 cm, preferably of at most 5 mm, particularly preferably of at most 3 mm and most preferably of at most 1 mm.

Within the scope of the present invention, a capillary gap is understood to denote a reaction space, which can be filled by means of capillary forces acting between the chamber support and the microarray. Usually, a capillary gap has a small thickness, for example of at most 1 mm, preferably of at most 750 μm and particularly preferably of at most 500 μm. According to the present invention, a thickness in the range of 10 to 300 μm, of 15 μm to 200 μm or of 25 μm to 150 μm is preferred as thickness of the capillary gap. In special embodiments of the present invention, the capillary gap has a thickness of 50 μm, 60 μm, 70 μm, 80 μm or 90 μm. According to the present invention, if the reaction space or the reaction chamber has a thickness of more than 2 mm, the reaction space or reaction chamber will not be referred to as a capillary gap anymore.

Within the scope of the present invention, a confocal fluorescence detection system is understood to denote a fluorescence detection system, wherein the object, i.e., the microarray, is illuminated in the focal plane of the objective by means of a point light source. Herein, point light source, object and point light detector are located on exactly optically conjugated planes. Examples for confocal systems are described in A. Diaspro, Confocal and 2-photon-microscopy: Foundations, Applications and Advances, Wiley-Liss, 2002.

Within the scope of the present invention, a fluorescence optical system imaging the entire volume of the reaction chamber is understood to denote a non-confocal fluorescence detection system, i.e., a fluorescence detection system, wherein the illumination by means of a point light source is not limited to the object, i.e., the microarray. Such a fluorescence detection system therefore has no focal limitation.

Conventional arrays or microarrays within the scope of the present invention comprise about 50 to 10,000, preferably 150 to 2,000 different species of probe molecules on a preferably square area of, for example, 1 mm to 4 mm×1 mm to 4 mm, preferably of 2 mm×2 mm. In further embodiments within the scope of the present invention, microarrays comprise about 50 to about 80,000, preferably about 100 to about 65,000, particularly preferably about 1,000 to about 10,000 different species of probe molecules on an area of several mm² to several cm², preferably about 1 mm² to 10 cm², particularly preferably 2 mm² to 1 cm² and most preferably about 4 mm² to 6.25 mm². For example, a conventional microarray has 100 to 65,000 different species of probe molecules on an area of 2 mm×2 mm.

Within the scope of the present invention, a microwell plate is understood to denote a layout of reaction vessels in a particular raster, which allows the automated performance of a multiplicity of biological, chemical, and lab-medical tests.

Within the scope of the present invention, a label or a marker is understood to denote a detectable unit, for example a fluorophor or an anchor group, whereto a detectable unit can be coupled.

Within the scope of the present invention, a sample or sample solution is the liquid to be analyzed with the nucleic acid molecules to be amplified and/or to be detected.

Within the scope of the present invention, a multiplication reaction or an amplification reaction usually comprises 10 to 50 or more amplification cycles, preferably about 20 to 40 cycles, particularly preferably about 30 cycles. Within the scope of the present invention, a cyclic amplification reaction preferably is a polymerase chain reaction (PCR).

Within the scope of the present invention, an amplification cycle denotes a single enhancement step of the cyclic amplification reaction. An enhancement step of the PCR is also referred to as PCR cycle.

Within the scope of the present invention, an amplification product denotes a product resulting from the enhancement or the multiplication or the amplification of the nucleic acid molecules to be amplified by means of the cyclic amplification reaction, preferably by means of the PCR. A nucleic acid molecule amplified by means of PCR is also referred to as PCR product.

Within the scope of the present invention, the denaturation temperature is understood to denote the temperature at which the double-stranded DNA is separated in the amplification cycle. Usually, the denaturation temperature, in particular in a PCR, is higher than 90° C., preferably about 95° C.

Within the scope of the present invention, the annealing temperature is understood to denote the temperature at which the primers hybridize to the nucleic acid to be detected. Usually, the annealing temperature, in particular in a PCR, lies within a range of 55° C. to 65° C. and preferably is about 60° C.

Within the scope of the present invention, the chain extension temperature or extension temperature is understood to denote the temperature at which the nucleic acid is synthesized by means of insertion of the monomer components. Usually, the extension temperature, in particular in a PCR, lies within a range of about 70° C. to about 75° C. and preferably is about 72° C.

Within the scope of the present invention, an oligonucleotide primer or primer denotes an oligonucleotide, which binds or hybridizes the DNA to be detected, also referred to as target DNA, wherein the synthesis of the complementary strand of the DNA to be detected in a cyclic amplification reaction starts from the binding site. In particular, primer denotes a short DNA or RNA oligonucleotide having preferably about 12 to 30 bases, which is complementary to a portion of a larger DNA or RNA molecule and has a free 3-OH group at its 3′-end. Due to said free 3′OH group, the primer can serve as substrate for any optional DNA or RNA polymerases, which synthesize nucleotides to the primer in 5′-3′direction. Herein, the sequence of the newly synthesized nucleotides is predetermined by that sequence of the template hybridized with the primer, which lies beyond the free 3′OH group of the primer. Primers of conventional length comprise between 12 and 50 nucleotides, preferably between 15 and 30 nucleotides.

A double-stranded nucleic acid molecule or a nucleic acid strand serving as template for the synthesis of complementary nucleic acid strands is usually referred to as template or template strand.

The formation of double-stranded nucleic acid molecules or duplex molecules from complementary single-stranded nucleic acid molecules is referred to as hybridization. Herein, the association preferably always occurs in pairs of A and T and G and C, respectively. Within the scope of hybridization, for example DNA-DNA duplexes, DNA-RNA duplexes, or RNA-RNA duplexes can be formed. By means of hybridization, duplexes with nucleic acid analogs can also be formed, like for example DNA-PNA duplexes, RNA-PNA duplexes, DNA-LNA duplexes, and RNA-LNA duplexes. Hybridization experiments are usually used for detecting the sequence complementarity and therefore the identity of two different nucleic acid molecules.

Within the scope of the present invention, processing is understood to denote purification, labeling, amplification, hybridization, and/or washing and rinsing steps as well as further procedure steps performed when detecting targets with the aid of substance libraries.

Within the scope of the present invention, a substrate substantially consisting of ceramic materials or a substrate substantially comprising ceramic materials is understood to denote a substrate comprising at least 75%, preferably at least 85%, and particularly preferably at least 90% ceramic materials.

Within the scope of the present invention, a substrate substantially consisting of aluminum oxide ceramics or a substrate substantially comprising aluminum oxide ceramics is understood to denote a substrate comprising at least 75%, preferably at least 85%, and particularly preferably at least 90% aluminum oxide ceramics.

Within the scope of the present invention, aluminum ceramics is understood to denote a ceramic material substantially consisting of aluminum oxide. Within the scope of the present invention, a ceramic material substantially consisting of aluminum oxide is understood to denote a ceramic material comprising at least 75%, preferably at least 85%, and particularly preferably at least 90% aluminum oxide.

Therefore, a first object of the present invention is, in particular, a device for amplifying and detecting nucleic acids, which comprises the following elements:

a) a temperature controlling and/or regulating unit;

b) a reaction chamber containing a support with a detection area, on which a compound library is immobilized, wherein the temperature in the reaction chamber can be controlled and/or regulated by means of the temperature controlling and regulating unit; and

c) an optical system, by means of which the time-dependent behavior of precipitate formations on the detection area is detectable.

In this aspect of the present invention, the integration of a temperature controlling and/or regulating unit, of a temperature-adjustable reaction chamber, and of an optical system, which ensures dynamic measurement of signal enhancement reactions by means of precipitation formation, in one device is an essential feature of the device according to the present invention.

In this aspect of the present invention, the device according to the present invention is characterized in that a detection of molecular interaction is also possible in manual operation due to the integrated optical system or reader system. This is particularly advantageous in fields like medical diagnostics. An exact determination of the relative quantitative amount of nucleic acids bound to the substance library is ensured by the fact that, in this embodiment, the device according to the present invention contains an integrated optical system, by means of which the time-dependent behavior of precipitation formations on the detection area is detectable.

The optical system ensures imaging of the substance library during or after completion of the amplification and/or detection reactions on a suitable detector, which is for example implemented in the form of a two-dimensional electrically readable detection element. In one embodiment of the device according to the present invention, the sample is illuminated by means of an illumination module or a light source of the optical system and the emerging signals are imaged in a filtered manner correspondingly to the labels used.

Furthermore, the optical system ensures a kinetic, i.e., dynamic, recording of the reaction results. In particular, the optical system of the device according to the present invention is suitable for recording the time-dependent behavior of a silver precipitation for the enhancement of hybridization signals between gold-labeled target molecules and the substance library. The highly integrated setup of the device according to the present invention allows the transfer of several images during the course of the reaction for processing in a suitable data processing module or controller.

The alterations originating from the time-dependent behavior of precipitation formation on the respective array elements can be evaluated in the device according to the present invention as is described in detail, inter alia, in the International Patent Application WO 02/02810. The respective contents of the International Patent Application WO 02/02810 are hereby also explicitly referred to.

For example, the device according to the present invention can be implemented in such a manner that the chamber body of the reaction chamber containing the chip with the detection area is sealingly applied to a chamber support in such a way that a sample space having a capillary gap between the chamber support and the detection area or the substrate of the chip is formed, whose temperature is adjustable and whose volume flow rate is controllable. This type of construction allows the performance of reactions, which only run efficiently within a particular range of temperature, and the preferably simultaneous detection of the reaction products by means of chip-based experiments.

Thus, the device according to the present invention can, for example, be used for amplifying the nucleic acid molecules by means of PCR and almost simultaneously detecting the PCR products by means of chip-based experiments. The sample liquid for such reactions can be efficiently heated or cooled by corresponding means for temperature regulation.

The device according to the present invention can also be used for performing a reverse transcriptase reaction and thereby transferring mRNA to cDNA and characterizing the reaction products by means of hybridization to the chip. In this manner, a so-called “gene profiling” can be performed. As both the reverse transcription and the hybridization are performed inside a chamber, this method is highly time-efficient and exhibits a low fault liability.

With the device according to the present invention, for example, a restriction digestion at desired temperatures can furthermore be performed inside the reaction chamber and the reaction products can be characterized by means of hybridization to a chip. Denaturation of the enzymes can be performed by means of heat deactivation. Thus, the device according to the present invention allows a time-efficient restriction-fragment-length-polymorphism mapping (RFLP mapping).

Furthermore, with the device according to the present invention, for example a ligation can also be performed.

With the device according to the present invention, the temperature-dependent melting behavior of nucleic acid target/nucleic acid probe complexes can furthermore be examined.

Furthermore, devices according to the present invention can be used for performing the temperature-dependent binding behavior of proteins. In this manner, it can for example be tested if antibodies are still capable of binding their respective antigens after a long period of heating. In this case, it is a prerequisite that the chip is not functionalized by nucleic acid molecules, but by the respective proteins or peptides.

The chamber body of the reaction chamber preferably consists of materials like glass, synthetic material and/or metals like high-grade steel, aluminum, and brass. For its manufacturing, for example synthetic materials suitable for injection molding can be used. Inter alia, synthetic materials like macrolon, nylon, PMMA, and teflon are conceivable. Alternatively, the reaction space between substance library support and chamber support can be closed by means of septa, which for example allow filling of the reaction space by means of syringes. In a preferred embodiment, the chamber body consists of optically transparent materials like glass, PMMA, polycarbonate, polystyrene, and/or topaz. Herein, the selection of materials is to be adjusted to the intended use of the device. For example, the temperatures the device will be exposed to are to be considered when selecting the materials. If, for example, the device shall be used for performing a PCR, for example only synthetic materials may be used, which remain stable for longer periods at temperatures like 95° C.

The chamber support preferably consists of glass, synthetic materials, silicon, metals, and/or ceramic materials. The chamber support can, for example, consist of aluminum oxide ceramics, nylon, and/or teflon.

In one embodiment, the chamber support consists of transparent materials like glass and/or optically transparent synthetic materials, for example PMMA, polycarbonate, polystyrene, or acrylic.

Preferably, the chamber support and/or the substrate is connected with means for temperature increase, which are integrated into the device according to the present invention, and should then preferably consist of materials having high thermal conductivity. Such thermally conductive materials offer the substantial advantage of ensuring a homogenous temperature profile covering the entire area of the reaction space and therefore temperature-dependent reactions like, for example, a PCR can be performed homogenously, with high yield, and controllably and/or regulably at great exactitude in the entire reaction chamber.

Thus, in a preferred embodiment, the chamber support and/or the substrate consist of materials having a high thermal conductivity, preferably a thermal conductivity in the range of 15 to 500 Wm⁻¹K⁻¹, particularly preferably in the range of 50 to 300 Wm⁻¹K⁻¹and most preferably in the range of 100 to 200 Wm⁻¹K⁻¹, wherein the materials are usually not optically transparent. Examples for suitable thermally conductive materials are silicon, ceramic materials like aluminum oxide ceramics, and/or metals like high-grade steel, aluminum, or brass.

In a particularly preferred embodiment, the substrate consists of materials having a high thermal conductivity, like for example ceramic materials. In this embodiment, the substrate is connected with a means for temperature increase, whereby the opposite side, the chamber support, can be made of a material not having a distinct thermal conductivity, like for example a material, which is also used for the remaining chamber body. Thus, as opposed to an embodiment wherein both the chamber support and the substrate are made of a cost-intensive material, a cost-intensive component is eliminated in this embodiment.

If the substrate or the support of the device according to the present invention substantially consist of ceramic materials, aluminum oxide ceramics are preferably used. Examples for such aluminum oxide ceramics are the ceramics A-473, A-476, and A-493 by Kyocera (Neuss, Germany). The ceramics substantially differ in their respective aluminum oxide content (A-473: 93%, A-476: 96%, and A-493: 99%) as well as in their surface roughness. Aluminum oxide ceramics having a surface roughness as low as possible are most preferably used.

Preferably, the chamber support and/or the substrate is equipped on its reverse side, i.e., the side facing away from the reaction chamber, with optionally miniaturized temperature sensors and/or electrodes or rather has heating structures in this place, so that tempering of the sample liquid as well as mixing of the sample liquid by means of an induced electro-osmotic flow is possible.

The temperature sensors can, for example, be implemented in the form of nickel-chromium thin film resistance temperature sensors.

The electrodes can, for example, be implemented in the form of gold-titanium electrodes and, in particular, in the form of a quadrupole.

The means for temperature increase can preferably be selected in such a way that fast heating and cooling of the liquid in the capillary gap is possible. Herein, fast heating and cooling is understood to signify that temperature alterations in a range of 0.2 K/s to 30 K/s, preferably of 0.5 K/s to 15 K/s, particularly preferably of 2 K/s to 15 K/s and most preferably of 8 K/s to 12 K/s or about 10 K/s can be mediated by the means for temperature increase. Preferably, temperature alterations of 1 K/s to 10 K/s can also be mediated by the means for temperature increase.

The means for temperature increase, for example in the form of heaters, can also be implemented in the form of nickel-chromium thin film resistance heaters, for example.

For further details on the specification and dimension of the temperature sensors, means for temperature increase, and the electrodes, it is referred to the contents of the International Patent Application WO 01/02094.

The chip or the substrate can preferably consist of borofloat glasses, silica glass, single-crystal CaF₂, sapphire discs, topaz, PMMA, polycarbonate, and/or polystyrene. The selection of materials is also to be adjusted according to the intended use of the device and/or the chip. If, for example, the chip is used for the characterization of PCR products, only materials, which can resist a temperature of 95° C., may be used.

Preferably, the chips are functionalized by nucleic acid molecules, in particular by DNA or RNA molecules. However, they can also be functionalized by peptides and/or proteins, like for example antibodies, receptor molecules, pharmaceutically active peptides, and/or hormones.

If the detection of the time-dependent behavior of precipitation formations on the detection area is performed in the dark field, i.e., if alterations of the dispersion properties of the detection area are detected, suitable materials for the substance library support are optically transparent materials like glass, particularly preferably borosilicate glass, and transparent polymers, like for example PMMA, polycarbonate, and/or acrylic; suitable materials for the chamber support are optically transparent materials like glass and/or synthetic materials and, in particular, optically not transparent materials like silicon, ceramic materials; suitable materials for the reaction chamber are synthetic materials like macrolon, PMMA, polycarbonate, teflon and the like, metals like high-grade steel, aluminum, and/or brass as well as glass. In the performance of dark field measurements of the device according to the present invention, the chamber support can alternatively consist of optically transparent materials, while the substance library support consists of optically not transparent materials.

In one embodiment, the device according to the present invention additionally comprises at least one fluid container, which is connected with the reaction chamber, and optionally a unit for controlling the loading and unloading of the reaction chamber with fluids. Within the scope of the present invention, fluids are understood to denote liquids and gases. The connection of the fluid containers with the reaction chamber can, for example, be implemented as is described in the International Patent Application WO 01/02094.

In a further embodiment, the device according to the present invention comprises a unit, which is connected with the optical system, for processing the signals recorded by the optical system. This coupling of detection unit and processing unit, which ensures the conversion of the reaction results into the analysis result, allows, inter alia, the use of the device according to the present invention as hand-held unit, for example in medical diagnostics.

Preferably, the device furthermore comprises an interface for external computers. This allows the transfer of data for storage purposes outside the device.

The optical system, by means of which the time-dependent behavior of precipitation formations on the detection area of the chip is detectable, preferably comprises a two-dimensionally readable detector. Preferably, the detector is a camera, in particular a CCD or CMOS camera or a similar camera. The use of cameras having electric image converters, like for example CCD or CMOS chips, allows the realization of high local resolutions.

The cameras used in the optical system of the device according to the present invention ensure that the illumination intensity is dispersed homogenously on the area to be imaged and that the signals to be detected can be imaged by means of reflection, transmission modulation, dispersion, polarization modulation and the like by means of the applied detection technique within the scope of the available dynamics. Such illumination methods are described, for example, in the International Patent Application WO 00/72018 and are commercially available (for example by Vision & Control GmbH (Suhl, Germany) for dark field illuminations and by Edmund Industrieoptik GmbH (Karlsruhe, Germany) for LED circular light).

A high local resolution of the area to be detected can, for example, also be achieved by imaging on detectors like mirror arrays or LCD elements and their adjustment according to a pattern to be detected or an area to be defined, as is for example described for fluorescence uses in the German laid-open patent application DE 199 14 279. The advantage of such a detector in measurement of reflection or transmission modulations is the integration of thermal, electric, and fluidic control and/or regulation, the possibility of optical signal processing and thus the lower technical demands made on the computer technology involved.

Normally, the detectors record the entire area of the probe array.

Alternatively, scanning detectors can also be used for reading out the chip. In the use of scanning point light sources and/or scanning detectors, the device according to the present invention comprises movable optical components for the direction of light and/or movable mechanical components for the attachment of the reaction chambers, so that directing the respective components across the individual positions to be scanned, i.e., the respective measurement points, is ensured. In this embodiment, image recording is performed by means of computational reconstruction of the image from the respective measurement points. In particular, the camera in this embodiment is a movable line camera.

Furthermore, the optical system preferably comprises in addition a light source, particularly preferably a multispectral or a coherent light source. Examples for light sources within the scope of the present invention are lasers, light emitting diodes (LED), and/or high pressure lamps. The light source of the optical system preferably ensures a homogenous illumination of the support.

In addition to point light sources, light sources in the form of illumination arrays may also be used in the device according to the present invention. In this implementation, a homogenous illumination of the support can, for example, also be ensured by the light source comprising several diffusely radiating light sources, whose overlay results in a homogenous illumination. Thus, for example diffusely dispersing LEDs, which are aligned in the form of a matrix, allow a homogenous illumination at short distances from the sample.

As already mentioned above, the device according to the present invention can be implemented in such a way that the detection area can be scanned in lines by the light source. If a raster-like or rather scanning direction of the light beam across the detection area is desired, the following embodiments of the device according to the present invention are conceivable:

For example, the detection area and accordingly the reaction chamber can be implemented in a movable manner and can be directed past a stationary light source. If the light source is a laser, the laser is in inoperative position herein. Furthermore, the detection area can be in inoperative position and a movable laser beam can be directed across the detection area. Finally, it is also possible that the light source is moved in an axis and the detection area is moved in another axis.

In another advantageous embodiment of the device according to the present invention, the device additionally comprises lenses, mirrors, and/or filters. The use of filters on the one hand allows spectral limitation of the homogenous illumination and on the other hand illumination of the samples with different wavelengths. In another variant, the device according to the present invention comprises filter changers. By means of said filter changers, the optical filters can be changed quickly and therefore possibly incorrect information, which for example occurs due to impurities, can be recognized unambiguously and can be eliminated.

As already mentioned above, the optical system is preferably developed in such a way that the detection area can be illuminated homogenously, preferably with an illumination intensity homogeneity of at least 50%, particularly preferably of at least 60% and most preferably of at least 70%.

In another preferred embodiment of the device according to the present invention, the optical system is developed in such a way that the time-dependent behavior of the alteration of transmission properties of the detection area is detectable. This can, for example, be ensured by light source and detector being arranged on opposite sides inside the reaction chamber and the reaction chamber including the support for the detection area being optically transparent at least in the region of the optical path leading from the light source to the detector.

In a further embodiment, the optical system is arranged in such a way that the time-dependent behavior of the alteration of reflection properties of the detection area is detectable. In a preferred embodiment for measuring the reflectivity, a surface mirror resides on the lower side of the support element. In this embodiment, the disadvantage of the poor reflection of the sample is supplemented by transmission effects, wherein the illumination light reflects via a mirror layer behind the sample, either in the form of an independent mirror or in the form of a layer applied to the back side of the sample support. Herein, for example, a planar emitter can be arranged on the side opposite of the support element and therefore also towards the sensor of, for example, a CCD camera. In this manner, a very compact adjustment is rendered possible. In a further preferred embodiment, in particular if the reflectivity of the support element is measured, the device additionally comprises a semi-transparent mirror between light source and support element. In this embodiment, the light of the light source reaches the sample through a semi-transparent mirror and the image is displayed on a camera in reflection through the semi-transparent mirror and, optionally, through an optical read-out system.

In a particularly preferred embodiment of the device according to the present invention, the optical system is arranged in such a way that the time-dependent behavior of the alteration of dispersion properties of the detection area is detectable. Herein, light source and detector are preferably arranged on the same side of the area to be detected. In this embodiment, the optical system can, for example, be arranged in such a way that the sample and/or the chip can be illuminated in a particular angle, which preferably is smaller than 45° and particularly preferably smaller than 30°. The illumination angle is selected in such a way that the incoming irradiated light, in the absence of local dispersion centers, i.e., before a precipitation formation on the detection area, is not directly reflected into the optical detection path and therefore no signal is detectable. If local dispersion centers occur on the detection area, for example due to formation of a precipitation, a part of the irradiated light reaches the optical detection path and therefore leads to a measurable signal in the optical system of the device according to the present invention.

Particularly preferably, the chamber support or the substance library support is optically not transparent in this embodiment, at least in the region of the detection area. Suitable optically not transparent materials are, for example, silicon, ceramic materials, or metals. The use of an optically not transparent chamber support has the advantage that, due to advantageous physical properties of the support materials, an easier, more exact and more homogenous temperature control of the reaction chamber is ensured, so that a successful performance of temperature-sensitive reactions like a PCR is warranted.

In a further preferred embodiment of the device according to the present invention, the optical excitation path of the light source is designed in such a way that regions of parallel light are present and therefore interference filters can be inserted into the optical system without displacing their transmission windows.

In a further preferred embodiment of the device according to the present invention, the optical detection path is designed in such a way that regions of parallel light are present, and therefore interference filters can be inserted into the optical system without displacing their transmission windows.

In non-parallel optical paths, interference filters strongly alter their spectral selectivity. If the optical system, due to the presence of regions of parallel light, allows the insertion or arrangement of interference filters in the device according to the present invention without altering their spectral selectivity, the device according to the present invention is also suitable for the detection of molecular interactions of substances labeled with fluorochromes. Thus, a universal CCD-based reaction and detection device can be implemented.

In this embodiment of the device according to the present invention, which is also suitable for the detection of fluorescence labeling, the optical system, with the use of white or multispectral light like, for example, halogen illumination, xenon, white light LED and the like, can for example have two filters in the optical illumination and detection path or, with the use of monochrome light sources like, for example, LED or laser, it can have, for example, one filter in the optical detection path.

In a further preferred embodiment, the reaction chamber is individually marked via a data matrix. To this end, when manufacturing the device according to the present invention, a data record containing information on the substance library, the performance of the detection reaction, and the like is stored in a database. Thus, the data record can in particular contain information on the layout of the probes on the array as well as information on how the evaluation is to be conducted in the most advantageous manner. The data record or the data matrix can further contain information on the temperature-time regime of a PCR, which is optionally to be performed for the amplification of the target molecules. The data record compiled in that manner is preferably equipped with a number, which is attached to the support in the form of the data matrix. By means of the number registered in the data matrix, the compiled data record can then optionally be accessed when reading out the substance library. Finally, the data matrix can be read out by the temperature controlling and/or regulating unit and by other controllers, like for example a control for loading and unloading of the reaction chamber, via the fluid containers and thus an automatic performance of amplification and detection reactions can be ensured.

In a further aspect of the present invention, a device for the amplification and detection of nucleic acids is provided, which also contains a temperature controlling and/or regulating unit as described above as well as a reaction chamber as described above comprising a support having a detection area, whereon a substance library is immobilized, wherein the temperature in the reaction chamber can be controlled and/or regulated by means of the temperature controlling and regulating unit.

In this aspect of the present invention, the device has electric contacts at the respective array spots instead of an optical system, however. These electric contacts can be contacted, for example, via electrodes. Due to the formation of a metallic precipitation on the array elements for signal enhancement of the, for example, gold-labeled targets bound to the substance library, a conductive material grows at those array spots, at which such a binding has occurred, which leads to an alteration of the local resistance. Thus, a modulation of particular electric parameters, like for example conductivity, resistance, and permeability, is possible via the electric contacts at the array spots.

Preferably, in this aspect of the present invention, the substance library support of the device according to the present invention has a three-dimensional structure, which is, for example, formed by bumps, base and/or through holes, whereby the effect in the melting of the growing conductive material is supported by the forming precipitate with the electric contacts and the alteration of electric parameters resulting therefrom.

The device according to the present invention based on optical detection preferably has a fluidics unit for the exchange of solutions in the reaction chamber, a temperature controlling and/or regulating unit as well as an optical system suitable for dynamic measurements. In a device-related sense, the above-mentioned units can optionally also be developed separately by means of implementation of corresponding interfaces.

In particular, substance libraries immobilized on the microarrays or chips are protein libraries like antibody, receptor protein, or membrane protein libraries, peptide libraries like receptor ligand libraries, libraries of pharmacologically active peptides or libraries of peptide hormones, and nucleic acid libraries like DNA or RNA molecule libraries. Particularly preferably, they are nucleic acid libraries.

As already mentioned above, the substance library preferably is immobilized on the substance library support or the detection area in the form of a microarray, particularly preferably having a density of 2 to 10,000 array spots per cm², most preferably having a density of 50 to 5,000 array spots per cm².

In all of the above-mentioned embodiments, the reaction chamber of the device according to the present invention is preferably developed in the form of a capillary gap. The capillary gap preferably has a thickness within a range of 10 μm to 200 μm, particularly preferably within a range of 25 μm to 150 μm and most preferably within a range of 50 μm to 100 μm. In special embodiments, the capillary gap has a thickness of 60 μm, 70 μm, 80 μm, or 90 μm.

In an alternative embodiment of the device according to the present invention, the reaction space or the reaction chamber has, for example, a thickness of 0.7 mm to 2.5 mm, preferably of 1.0 mm to 2.0 mm, and particularly preferably of 1.2 mm to 1.8 mm. In a special embodiment, the thickness of the reaction space is 1.5 mm.

Furthermore, in all of the above-described embodiments of the device according to the present invention, a pre-amplification of the material to be analyzed is not required. From the sample material extracted from bacteria, blood, or other cells, specific partitions can be amplified and hybridized to the support with the aid of a PCR (polymerase chain reaction), in particular in the presence of the device according to the present invention or the substance library support as described in DE 102 53 966. This signifies a substantial reduction of labor expenditure.

Thus, the device according to the present invention is particularly suitable for the use in parallel performance of amplification of the target molecules to be analyzed by means of PCR and the detection by means of hybridization of the target molecules with the substance library support. Herein, the nucleic acid to be detected is first amplified by means of a PCR, wherein preferably at least one competitor inhibiting the formation of one of the two template strands amplified by means of the PCR is added to the reaction in the beginning. In particular, a DNA molecule, which competes against one of the primers used for the PCR amplification of the template for binding to the template and which can not be extended enzymatically, is added to the PCR. The single-stranded nucleic acid molecules amplified by means of the PCR are then detected by means of hybridization with a complementary probe. Alternatively, the nucleic acid to be detected is first amplified in single strand surplus by means of a PCR and is detected by means of a subsequent hybridization with a complementary probe, wherein a competitor, which is a DNA molecule or a molecule of a nucleic acid analog capable of hybridizing to one of the two strands of the template but not to the region detected by means of probe hybridization and which cannot be extended enzymatically, is added to the PCR reaction at the beginning.

Every molecule causing a preferred amplification of only one of the two template strands present in the PCR reaction can be used as competitor in the PCR. According to the present invention, competitors can be proteins, peptides, DNA ligands, intercalators, nucleic acids, or analogs thereof. According to the present invention, proteins or peptides, which are capable of binding single-stranded nucleic acids with sequence specificity and which have the above-defined properties, are preferably used as competitors. Particularly preferably, nucleic acid molecules and nucleic acid analog molecules are used as secondary structure breakers.

The formation of one of the two template strands is substantially inhibited by initial addition of the competitor to the PCR during the amplification. “Substantially inhibited” means that within the scope of the PCR a single strand surplus and an amount of the other template strand are produced, which suffice to allow an efficient detection of the amplified strand by means of the hybridization. Therefore, the amplification does not follow exponential kinetics of the form 2ⁿ (with n=number of cycles), but rather attenuated amplification kinetics of the form <2ⁿ.

The single strand surplus obtained by means of the PCR in relation to the non-amplified strand has the factor 1.1 to 1,000, preferably the factor 1.1 to 300, also preferably the factor 1.1 to 100, particularly preferably the factor 1.5 to 100, also particularly preferably the factor 1.5 to 50, in particular preferably the factor 1.5 to 20, and most preferably the factor 1.5 to 10.

Typically, the function of a competitor will be to bind selectively to one of the two template strands and therefore to inhibit the amplification of the corresponding complementary strand. Therefore, competitors can be single-stranded DNA- or RNA-binding proteins having specificity for one of the two template strands to be amplified in a PCR. They can also be aptamers sequence-specifically binding only to specific regions of one of the two template strands to be amplified.

Nucleic acids or nucleic acid analogs are preferably used as competitors in the method according to the present invention. Conventionally, the nucleic acids or nucleic acid analogs will act as competitors of the PCR by either competing against one of the primers used for the PCR for the primer binding site or by being capable of hybridizing with a region of a template strand to be detected due to a sequence complementarity. This region is not the sequence detected by the probe. Such nucleic acid competitors are enzymatically not extendable.

The nucleic acid analogs can be e.g., so-called peptide nucleic acids (PNA). However, nucleic acid analogs can also be nucleic acid molecules, in which the nucleotides are linked to one another via a phosphothioate bond instead of a phosphate bond. They can also be nucleic acid analogs, wherein the naturally occurring sugar components ribose or deoxyribose have been replaced with alternative sugars like e.g., arabinose or trehalose. Furthermore, the nucleic acid derivative can be “locked nucleic acid” (LNA). Further conventional nucleic acid analogs are known to the person skilled in the art.

DNA or RNA molecules, in particular preferably DNA or RNA oligonucleotides or analogs thereof, are preferably used as competitors.

Depending on the sequence of the nucleic acid molecules or nucleic acid analogs used as competitors, the inhibition of the amplification of one of the two template strands within the scope of the PCR reaction is based on different mechanisms. By way of the example of a DNA molecule, this is discussed in the following.

If, for example, a DNA molecule is used as competitor, it can have a sequence, which is at least partially identical to the sequence of one of the primers used for the PCR in such a way that a specific hybridization of the DNA competitor molecule with the corresponding template strand is possible under stringent conditions. As, according to the present invention, the DNA molecule used for competition in this case is not extendable by means of a DNA polymerase, the DNA molecule competes for binding to the template against the respective primer during the PCR reaction. Depending on the ratio of the DNA competitor molecule and the primer, the amplification of the template strand defined by the primer can thus be inhibited in such a way that the production of this template strand is significantly reduced. Herein, the PCR proceeds according to exponential kinetics higher than would be expected with respect to the amounts of competitors used. In this manner, a single strand surplus emerges in an amount, which is sufficient for the efficient detection of the amplified target molecules by means of hybridization.

In this embodiment, the nucleic acid molecules or nucleic acid analogs used for competition must not be enzymatically extendable. “Enzymatically not extendable” means that the DNA or RNA polymerase used for the amplification cannot use the nucleic acid competitor as primer, i.e., it is not capable of synthesizing the corresponding opposite strand of the template 3′ from the sequence defined by the competitor.

Alternatively to the above-depicted possibility, the DNA competitor molecule can also have a sequence complementary to a region of the template strand to be detected, which is not addressed by one of the primer sequences and which is enzymatically not extendable. Within the scope of the PCR, the DNA competitor molecule will then hybridize to this template strand and correspondingly block the amplification of this strand.

The person skilled in the art knows that the sequences of DNA competitor molecules or generally nucleic acid competitor molecules can be selected correspondingly. If the nucleic acid competitor molecules have a sequence, which is not substantially identical to the sequence of one of the primers used for the PCR, but is complementary to another region of the template strand to be detected, this sequence is to be selected in such a way that it does not fall within the region of the template sequence, which is detected with a probe within the scope of the hybridization. This is necessary because there is no need to have a processing reaction between the PCR and the hybridization reaction. If a nucleic acid molecule, which falls within the region to be detected, were used as competitor, it would compete for binding to the probe against the single-stranded target molecule.

Such competitors preferably hybridize near the template sequence detected by the probe. Herein, the position specification “near” is to be understood according to the present invention, in the same way as given for secondary structure breakers. However, the competitors according to the present invention can also hybridize in the immediate proximity of the sequence to be detected, i.e., in exactly one nucleotide's distance from the target sequence to be detected.

If enzymatically not extendable nucleic acids or nucleic acid analogs are used as competing molecules, they are to be selected according to their sequence and structure in such a way that they cannot be enzymatically extended by DNA or RNA polymerases. Preferably, the 3′-end of a nucleic acid competitor is designed in such a way that it has no complementarity to the template and/or has at its 3′-end another substituent instead of the 3-OH group.

If the 3′ end of the nucleic acid competitor has no complementarity to the template, regardless of whether the nucleic acid competitor binds to one of the primer binding sites of the template or to one of the sequences of the template to be amplified by means of the PCR, the nucleic acid competitor cannot be extended by the conventional DNA polymerases due to the lack of base complementarity at its 3′-end. This type of non-extensibility of nucleic acid competitors by DNA polymerases is known to the person skilled in the art. Preferably, the nucleic acid competitor has no complementarity to its target sequence at its 3′-end with respect to the last 4 bases, particularly preferably to the last 3 bases, in particular preferably to the last 2 bases and most preferably to the last base. In the mentioned positions, such competitors can also have non-natural bases, which do not allow hybridization.

Nucleic acid competitors, which are enzymatically not extendable, can also have a 100%-complementarity to their target sequence, if they are modified in their backbone or at their 3′-end in such a way that they are enzymatically not extendable.

If the nucleic acid competitor has at its 3′-end a group other than the OH group, these substituents are preferably a phosphate group, a hydrogen atom (dideoxynucleotide), a biotin group, or an amino group. These groups cannot be extended by the conventional polymerases.

The use of a DNA molecule, which competes for binding to the template against one of the two primers used for the PCR and which was provided with an amino link at its 3′-end during chemical synthesis, as a competitor in such a method is particularly preferred. Such competitors can have a 100% complementary to their target sequence.

However, nucleic acid competitors, like for example PNAs do not need to have a blocked 3′ OH group or a non-complementary base at their 3′-end as they are not recognized by the DNA polymerases because of the backbone modified by the peptide bond and thus they are not extended. Other corresponding modifications of the phosphate group, which are not recognized by the DNA polymerases, are known to the person skilled in the art. Belonging thereto are, inter alia, nucleic acids having backbone modifications, like for example 2′-5′ amide bonds (Chan et al. (1999) J. Chem. Soc., Perkin Trans. 1, 315-320), sulfide bonds (Kawai et al. (1993) Nucleic Acids Res., 1 (6), 1473-1479), LNA (Sorensen et al. (2002) J. Am. Chem. Soc., 124 (10), 2164-2176) and TNA (Schoning et al. (2000) Science, 290 (5495), 1347-1351).

Several competitors hybridizing to different regions of the template (for example the primer binding site, inter alia) can also simultaneously be used in a PCR. The efficiency of the hybridization can additionally be increased, if the competitors have properties of secondary structure breakers.

In an alternative embodiment, the DNA competitor molecule can also have a sequence complementary to one of the primers. Depending on the ratio of antisense DNA competitor molecule and primer, such for example antisense DNA competitor molecules can then be used to titrate the primer in the PCR reaction, so that it will no longer hybridize with the corresponding template strand and, correspondingly, only the template strand defined by the other primer is amplified. The person skilled in the art is aware of the fact that, in this embodiment of the invention, the nucleic acid competitor can, but does not have to, be enzymatically extendable.

If, within the scope of the present invention, it is talked about nucleic acid competitors, this includes nucleic acid analog competitors, unless a different meaning arises from the respective context. The nucleic acid competitor can bind to the corresponding strand of the template reversibly or irreversibly. The binding can take place by means of covalent or non-covalent interactions.

Preferably, binding of the nucleic acid competitor takes place via non-covalent interactions and is reversible. In particular preferably, binding to the template takes place via formation of Watson-Crick base pairings.

The sequences of the nucleic acid competitors normally adapt to the sequence of the template strand to be detected. In the case of antisense primers, though, they adapt to the primer sequences to be titrated, which are in turn defined by the template sequences, however.

PCR amplification of nucleic acids is a standard laboratory method, whose various possibilities of variation and development are familiar to the person skilled in the art. In principle, a PCR is characterized in that the double-stranded nucleic acid template, usually a double-stranded DNA molecule, is first subjected to heat denaturation for 5 minutes at 95° C., whereby the two strands are separated from each other. After cooling down to the so-called “annealing” temperature (defined by the primer with the lower melting temperature), the “forward” and “reverse” primers present in the reaction solution accumulate at those sites in the respective template strands, which are complementary to their own sequences. Herein, the “annealing” temperature of the primers adapts to the length and base structure of the primers. It can be calculated on the basis of theoretical considerations. Information on the calculation of “annealing” temperatures can be found, for example, in Sambrook et al. (vide supra).

Annealing of the primers, which typically is performed within a range of temperature between 40 to 75° C., preferably between 45 to 72° C. and in particular preferably between 50 to 72° C., is followed by an elongation step, wherein deoxyribonucleotides are linked with the 3′-end of the primers by the activity of the DNA polymerase present in the reaction solution. Herein, the identity of the inserted dNTPs depends on the sequence of the template strand hybridized with the primer. As normally thermostable DNA polymerases are used, the elongation step usually runs at between 68 to 72° C.

In a symmetrical PCR, an exponential increase of the nucleic acid region of the target defined by the primer sequences is achieved by means of repeating this described cycle of denaturation, annealing and elongation of the primers. With respect to the buffer conditions of the PCR, the usable DNA polymerases, the production of double-stranded DNA templates, the design of primers, the selection of the annealing temperature, and variations of the classic PCR, the person skilled in the art has numerous works of literature at his disposal.

It is familiar to the person skilled in the art that also, for example, single-stranded RNA, like for example mRNA, can be used as template. Usually, it is previously transcribed into a double-stranded cDNA by means of a reverse transcription.

In a preferred embodiment, a thermostable DNA-dependent polymerase is used as polymerase. In a particularly preferred embodiment, a thermostable DNA-dependent DNA polymerase is used, which is selected from the group consisting of Taq-DNA polymerase (Eppendorf, Hamburg, Germany and Qiagen, Hilden, Germany), Pfu-DNA polymerase (Stratagene, La Jolla, USA), Tth-DNA polymerase (Biozym Epicenter Technol., Madison, USA), Vent-DNA polymerase, DeepVent-DNA polymerase (New England Biolabs, Beverly, USA), Expand-DNA polymerase (Roche, Mannheim, Germany).

The use of polymerases, which have been optimized from naturally occurring polymerases by means of specific or evolutive alteration, is also preferred. When performing the PCR in the presence of the substance library support, the use of the Taq-polymerase by Eppendorf (Germany) or of the Advantage cDNA Polymerase Mix by Clontech (Palo Alto, Calif., USA) is particularly preferred.

In a further aspect of the present invention, a method for the detection of nucleic acids is provided, which comprises the following steps:

a) providing a device according to the present invention, as described above;

b) interaction of the nucleic acid to be detected with the substance library immobilized on the detection area; and

c) detecting said interaction.

The targets to be examined can be available in every type of sample, preferably in a biological sample.

Preferably, the targets are isolated, purified, copied, and/or amplified by the method of the present invention before their detection and quantification.

Usually, the amplification is performed by means of conventional PCR methods or by means of a method for the parallel performance of amplification of the target molecules to be analyzed by means of PCR and detection by means of hybridization of the target molecules with the substance library support, as is described above.

In a further embodiment, the amplification is performed as a multiplex PCR in a two-step process (see also WO 97/45559). In a first step, a multiplex PCR is performed by way of using fusion primers, whose 3′-ends are gene specific and whose 5′-ends are universal regions. The latter are the same in all forward and reverse primers used in the multiplex reaction. In this first step, the primer amount is limiting. Hereby, all multiplex products can be amplified until a uniform molar level is achieved, given that the number of cycles is adequate for reaching primer limitation for all products. In a second step, universal primers identical to the 5′-regions of the fusion primers are present. Amplification is performed until the desired amount of DNA is obtained.

In the method according to the present invention, the detection is preferably performed in such a way that the bound targets are linked to at least one label, which is detected in step c).

As already mentioned above, the label coupled to the targets or probes preferably is a detectable unit or a detectable unit coupled to the targets or probes via an anchor group. Concerning the possibilities for detection and/or labeling, the method according to the present invention is highly flexible. Thus, the method according to the present invention is compatible with a variety of physical, chemical, or biochemical detection methods. The only prerequisite is that the unit or structure to be detected can directly be coupled, and/or can be linked via an anchor group, which can be coupled with the oligonucleotide, to a probe or a target, for example an oligonucleotide.

The detection of the label can be based on fluorescence, magnetism, charge, mass, affinity, enzymatic activity, reactivity, a gold label, and the like. Thus, the label can, for example, be based on the use of fluorophore-labeled structures or components. In connection with the fluorescence detection, the label can be an arbitrary colorant, which can be coupled to targets or probes during or after their synthesis. Examples are Cy colorants (Amersham Pharmacia Biotech, Uppsala, Sweden), Alexa colorants, Texas Red, Fluorescein, Rhodamin (Molecular Probes, Eugene, Oreg., USA), lanthanides like samarium, ytterbium, and europium (EG&G, Wallac, Freiburg, Germany).

Besides fluorescence markers, also luminescence markers, metal markers, enzyme markers, radioactive markers, and/or polymeric markers can be used within the scope of the present invention as labeling and/or detection unit, which is coupled to the targets or the probes.

Likewise, a nucleic acid can be used as label (tag), which can be detected by means of hybridization with a labeled reporter (sandwich hybridization). Diverse molecular biological detection reactions like primer extension, ligation, and RCA are used for the detection of the tag.

In an alternative embodiment of the method according to the present invention, the detectable unit is coupled with the targets or probes via an anchor group. Preferably used anchor groups are biotin, digoxigenin, and the like. In a subsequent reaction, the anchor group is converted with specifically binding components, for example streptavidin conjugates or antibody conjugates, which in turn are detectable or trigger a detectable reaction. With the use of anchor groups, the conversion of the anchor groups to detectable units can be performed before, during, or after the addition of the sample comprising the targets, or, optionally, before, during, or after the cleavage of the selectively cleavable bond in the probes.

According to the present invention, the labeling can also be performed by means of interaction of a labeled molecule with the probe molecules. For example, the labeling can be performed by means of hybridization of a labeled oligonucleotide with an oligonucleotide probe or an oligonucleotide target, as described above.

Further labeling methods and detection systems suitable within the scope of the present invention are described, for example, in Lottspeich and Zorbas, Bioanalytik, Spektrum Akademischer Verlag, Heidelberg, Berlin, 1998, chapter 23.3 and 23.4.

In a preferred embodiment of the method according to the present invention, detection methods are used, which in result yield an adduct having a particular solubility product, which leads to a precipitation. For labeling, in particular substrates or educts are used, which can be converted to a hardly soluble, usually stained product. In this labeling reaction, for example, enzymes can be used, which catalyze the conversion of a substrate to a hardly soluble product. Reactions suitable for leading to a precipitation at the array elements as well as possibilities for the detection of the precipitation are, for example, described in the International Patent Application WO 00/72018 and in the International Patent Application WO 02/02810, whose contents are hereby explicitly referred to.

In a particularly preferred embodiment of the method according to the present invention, the bound targets are equipped with a label catalyzing the reaction of a soluble substrate or educt to form a hardly soluble precipitation at the array element, where a probe/target interaction has occurred or acting as a crystal nucleus for the conversion of a soluble substrate or educts to a hardly soluble precipitation at the array element, where a probe/target interaction has occurred.

In this manner, the use of the method according to the present invention allows the simultaneous qualitative and quantitative analysis of a variety of probe/target interactions, wherein individual array elements having a size of ≦1000 μm, preferably of ≦100 μm, and particularly preferably of ≦50 μm can be implemented.

The use of enzymatic labels is known in immunocytochemistry and in immunological tests based on microwell plates (see E. Lidell and I. Weeks, Antibody Technology, BIOS Scientific Publishers Limited, 1995). Thus, for example, enzymes catalyze the conversion of a substrate to a hardly soluble, usually stained product.

Particularly preferably, the reaction leading to precipitation formation at the array elements is a conversion of a soluble substrate or educt to a hardly soluble product catalyzed by an enzyme. In a special embodiment, the reaction leading to precipitation formation at the array elements is an oxidation of 3,3′,5,5′-tetramethylbenzidine catalyzed by a peroxidase.

Horseradish peroxidase is preferably used for the oxidation of 3,3′,5,5′-tetramethylbenzidine. However, the person skilled in the art knows further peroxidases, which can be used for the oxidation of 3,3′,5,5′-tetramethylbenzidine.

It is assumed that 3,3′,5,5′-tetramethylbenzidine, under the catalytic effect of a peroxidase, is oxidized in a first step to form a blue-stained radical cation (see for example Gallati and Pracht, J. Clin. Chem. Clin. Biochem. 1985, 23, 8, 454). This blue-stained radical cation is precipitated in the form of a complex by means of a polyanion, like for example dextran sulfate. The precipitation reaction by means of peroxidase-catalyzed oxidation of 3,3′,5,5′-tetramethylbenzidine is, for example, described in EP 0 456 782.

Without claiming to be complete, the following Table 1 gives a survey of several reactions possibly suitable for leading to a precipitation at array elements, where an interaction between target and probe has occurred:

TABLE 1

| catalyst or | |
|---|---|
| crystal nucleus | substrate or educt |
| horseradish peroxidase | DAB (3,3′-diaminobenzidine) |
| | 4-CN (4-chloro-1-naphthol) |
| | AEC (3-amino-9-ethylcarbazole) |
| | HYR (p-phenylenediamine-HCl and |
| | pyrocatechol) |
| | TMB (3,3′,5,5′-tetramethylbenzidine) |
| | naphthol/pyronin |
| alkaline phosphatase | brom-chlor-indoyl-phosphate (BCIP) and |
| | nitroblue tetrazolium (NBT) |
| glucose oxidase | t-NBT and m-PMS |
| | (nitroblue tetrazolium chloride and |
| | phenazine methosulfate) |
| gold particles | silver nitrate |
| | silver tartrate |

The labeling of biological samples with enzymes or gold, in particular nanocrystalline gold, has been sufficiently described (see, inter alia, F. Lottspeich and H. Zorbas, Bioanalytik, Spektrum Akademischer Verlag, Heidelberg, Berlin, 1998; E. Lidell and I. Weeks, Antibody Technology, BIOS Scientific Publishers Limited, 1995).

Further possibilities for the detection of the probe/target interactions via insoluble precipitates in the method according to the present invention are described in: Immunogold-Silver Staining, Principles, Methods and Applications, Hrsg.: M. A. Hayat, 1995, CRC Press; Eur J Immunogenet February-April 1991; 18(1-2):33-55 HLA-DR, DQ and DP typing using PCR amplification and immobilized probes. Erlich H, Bugawan T, Begovich A B, Scharf S, Griffith R, Saiki R, Higuchi R, Walsh P S. Department of Human Genetics, Cetus Corp., Emeryville, Calif. 94608; Mol Cell Probes June 1993; 7(3):199-207 A combined modified reverse dot-blot and nested PCR assay for the specific non-radioactive detection of Listeria monocytogenes. Bsat N, Batt C A. Department of Food Science, Cornell University, Ithaca, N.Y. 14853. Immunogenetics 1990;32(4):231-41 Erratum in: Immunogenetics 1991;34(6):413 Rapid HLA-DPB typing using enzymatically amplified DNA and nonradioactive sequence-specific oligonucleotide probes. Bugawan T L, Begovich A B, Erlich HA. Department of Human Genetics, Cetus Corporation, Emeryville, Calif. 94608. Hum Immunol December 1992; 35(4):215-22 Generic HLA-DRB1 gene oligotyping by a nonradioactive reverse dot-blot methodology. Eliaou J F, Palmade F, Avinens O, Edouard E, Ballaguer P, Nicolas J C, Clot J. Laboratory of Immunology, Saint Eloi Hospital, CHU Montpellier, France. J Immunol Methods Nov. 30, 1984;74(2):353-60 Sensitive visualization of antigen-antibody reactions in dot and blot immune overlay assays with immunogold and immunogold/silver staining. Moeremans M, Daneels G, Van Dijck A, Langanger G, De Mey J. Histochemistry 1987;86(6):609-15 Non-radioactive in situ hybridization. A comparison of several immunocytochemical detection systems using reflection-contrast and electron microscopy. Cremers A F, Jansen in de Wal N, Wiegant J, Dirks R W, Weisbeek P, van der Ploeg M, Landegent J E.

Within the scope of the present invention, the following variants, inter alia, are conceivable for the detection of the probe/target interactions via insoluble precipitates in the methods according to the present invention.

In one embodiment of the methods according to the present invention, the targets are equipped with a catalyst, preferably an enzyme, which catalyzes the conversion of a soluble substrate or educt to an insoluble product. In this case, the reaction leading to precipitation formation at the array elements is a conversion of a soluble substrate or educt to an insoluble product in the presence of a catalyst coupled with one of the targets, preferably an enzyme. Preferably, the enzyme is selected from the group consisting of horseradish peroxidase, alkaline phosphatase, and glucose oxidase. The soluble substrate or educt is preferably selected from the group consisting of 3,3′-diaminobenzidine, 4-chloro-1-naphthol, 3-amino-9-ethylcarbazole, p-phenylenediamine-HCl/pyrocatechol, 3,3′,5,5′-tetramethylbenzidine, naphthol/pyronin, brom-chlor-indoyl-phosphate, nitroblue tetrazolium, and phenazine methosulfate. For example, a colorless soluble hydrogen donor, for example 3,3′-diaminobenzidine, is converted to an insoluble stained product in the presence of hydrogen peroxide. The enzyme horseradish peroxidase transfers hydrogen ions from the donors to hydrogen peroxide while forming water.

Further possibilities of the detection of probe/target interactions via insoluble precipitates are, in particular, described in WO 02/02810.

In a preferred embodiment of the present invention, the reaction leading to precipitation formation at the array elements is the formation of a metallic precipitation. Particularly preferably, the reaction leading to precipitation formation at the array elements is the chemical reduction of a silver compound, preferably silver nitrate, silver lactate, silver acetate, or silver tartrate, to form elemental silver. Formaldehyde and/or hydroquinone are preferably used as reducing agents.

Thus, a further possibility for the detection of molecular interaction on arrays is the use of metal labels. Herein, for example colloidal gold or defined gold clusters are coupled with the targets, optionally via particular mediator molecules like streptavidin. The staining resulting from gold labeling is preferably enhanced by the subsequent reaction with less precious metals, like for example silver, wherein the gold label coupled with the targets acts as crystal nucleus or catalyst, for example, for the reduction of silver ions to a silver precipitate. The targets coupled with gold labels are also referred to as gold conjugates in the following.

In this embodiment of the method according to the present invention, a relative quantification of the probe/target interaction can also be performed. The relative quantification of the concentration of the bound targets on a probe array by means of detection of a precipitate is performed via the concentration of the labels coupled with the targets, which catalyze the reaction of a soluble substrate to form a hardly soluble precipitate on the array element, where a probe/target interaction has occurred or which act as crystal nucleus for such reactions. For example in the case of oligonucleotide probes labeled with nanogold and purified with HPLC, the ratio of bound target to gold particles is 1: 1. In other embodiments of the present invention, the ratio can be a multiplicity or also a fraction thereof.

Thus, in this embodiment of the detection method according to the present invention, the detection is performed by means of measuring the transmission alteration, reflection, or dispersion caused by the precipitate, which is generated by the catalytic effect of the label coupled with the bound targets on the array elements, where a probe/target interaction has occurred.

In the case of coupling colloidal gold or defined gold clusters with the targets, light absorption is already evoked by the presence of these metallic labels. In order to enhance the light absorption, however, a non-transparent precipitate is precipitated preferably catalytically by such interactive hybrids, i.e., targets equipped with a label like, for example, colloidal gold or defined gold clusters. In the case of gold conjugates, the use of silver as precipitate has turned out to be particularly preferable.

Thus, in a further preferred embodiment of the method according to the present invention, the time-dependent behavior of the precipitation formation at the array elements is detected in the form of signal intensities in step c). In this manner, an exact determination of the relative quantitative amount of targets bound can be ensured. Such a procedure is described in detail in the International Patent Application WO 02/02810, whose contents are hereby explicitly referred to.

In the following, the qualitative and/or quantitative detection of the probe/target interaction by means of measuring the absorption of transmitted light is explained by way of an example. Of course, the procedure described in the following is not limited to the above-described silver/gold staining, but can correspondingly be applied to all detection reactions, wherein the bound targets are equipped with a label catalyzing the reaction of a soluble substrate or educt to form a hardly soluble precipitation on the array element, where a probe/target interaction has occurred, or rather which acts as a crystal nucleus for the conversion of a soluble substrate to a hardly soluble precipitation on the array element, where a probe/target interaction has occurred.

At first, the target molecule is biotinilated, for example, by means of PCR. The PCR product is hybridized against a substance library, for example a DNA library. Subsequently, streptavidin-functionalized gold beads, which react with the biotinilated hybrids, for example DNA hybrids, are added to the reaction chamber. By means of, for example, reducing silver nitrate with hydroquinone under the catalytic effect of gold, a silver precipitate can be generated at the gold beads, which are now specifically bound at the surface (see, inter alia, WO 00/72018, DE 100 33 334.6, M. A. Hayat, Immunogold-Silver Staining, CRC Press, New York, 1995).

Light absorption by the silver precipitate depends on the amount of silver precipitated. Thus, the light intensity, which radiates through the precipitate, can be calculated according to a function similar to the Lambert-Beer law:
I=I₀*exp (−a*b) (I)

Herein, I is the light intensity after the absorption, I₀ is the light intensity before the absorption, a is an absorption coefficient multiplied by the shading per area unit b by the silver precipitate. Intensity I and time t are available as measured quantities. These measured quantities are obtained by means of illuminating the support element with the substance library and recording the transmitted light by means of a camera. This recording is repeated at regular intervals, while the silver precipitation is performed. The brightness values of the individual library regions (spots) are evaluated for each recording, whereby the intensity I of each spot is maintained. By means of standard software, like for example IconoClust® (Clondiag, Jena, Germany), these brightness values can be calculated automatically. Measurement curves are obtained by means of plotting I/I₀ against the time t. In the following manner, the Lambert-Beer law, corresponding to equation (I), can be brought in connection with the time-dependent silver precipitation sequences obtained in this way.

The area F, which is shaded by one silver bead, is:
F=π*r² (II)

As the precipitation speed per area element for one spot can be assumed constant, the radius r of the silver beads also grows at a constant speed:
r=dr/dt*t (III)

The shading per area unit b by the silver precipitate is proportional to the number of silver beads per area unit N, to the shading area per silver bead F, and to a constant k.
b=N*F*k (III)

Thus, the function for the intensity I in dependency on the time t is calculated with
I=I₀*exp(−a′*t²) (IV)

wherein a′ is an unknown composite silver absorption constant.

For each silver precipitation reaction an individual precipitation rate must be assumed; likewise also for the unspecific reaction at the surface:
I=I₀*exp(−a′_{H}*t²), (V)

wherein a′_{H} is the unspecific silver absorption constant.

At each spot i, both a specific precipitation reaction and an unspecific precipitation reaction occur:
Iᵢ=I₀ᵢ*(exp(−a′ᵢ*t²)+exp (−a′_{H}*t²) )+Oᵢ, (VI)

wherein O is a device-relevant offset value.

The number of gold beads being precipitated per area unit depends, on the one hand, on the amount of targets labeled with gold beads and, on the other hand, on the binding strength of the targets, for example the target DNA with the probes, for example the spot DNA. If no target, which interacts with a probe on the respective array element, is present in the sample, the precipitation of gold beads on the surface of this array element or spot will not occur. If the bond between probe and target is weak, only very few gold beads will deposit at the surface of this array element.

As b and therefore a′ are directly proportional to the number of gold beads and therefore silver beads N, a′ is a measure for the concentration of the target DNA and the binding strength of the target DNA at the array element or spot i.

By means of non-linear regression with the aid of equation (VI), the silver absorption constant a′ can be calculated from the measurement curves obtained. The calculation of the constant a′ can be used as significant measured quantity for the binding strength and the concentration of the target DNA at a spot. Furthermore, the time constant T of the precipitation reaction can be determined from the constant a′:
τₗ=(1/α′ᵢ)^{0.5} (VII)

I₀ and O can be determined as further parameters by means of regression. In homogeneities of illumination of the DNA library can be corrected by means of the light intensity I₀. Alternatively, a flat field correction of all further images, which were recorded for the time-dependent sequence at a later point in time, can be conducted via the image of the entire DNA library at a point in time t=0. By means of the parameter O, the validity of a measurement can be estimated.

As the correct adaptation of an exponential function to the measurement values requires a non-linear regression algorithm, for example a non-linear regression according to Marquardt (H. R. Schwarz, Numerische Mathematik, Teubner Verlag, Stuttgart, Germany, 1998), it can be advantageous to fall back upon a less exact, but instead more robust and therefore more linear method for determining the measurement values from the time-dependent sequences. To this end, the time values are squared and the intensity measurement values are logarithmized. The values obtained in this way are then adapted to a linear equation. The regression parameters obtained this way can be used as measurement value and for testing the validity. When using a linear method, illumination in homogeneities cannot be corrected anymore via I₀, there remains, however, the possibility of a flat field correction.

A further variant of evaluation is to directly use the gray values of the individual spots as measurement values after a determined period. However, this method has the disadvantages that it cannot be assessed in advance which point in time is optimal for evaluation and that the measurement values exhibit a lower statistical certainty. Moreover, a possible illumination in homogeneity can only be performed via a flat field correction.

A further aspect of the present invention relates to a method for the amplification and the qualitative and quantitative detection of nucleic acids in a sample, comprising the following steps:

a) inserting the sample into a reaction chamber, which is formed between a chamber support and a microarray, wherein the microarray comprises a substrate, on which nucleic acid probes are immobilized on array elements;

b) amplifying the nucleic acids to be detected in the reaction chamber by means of a cyclic amplification reaction;

c) detecting a hybridization between the nucleic acids to be detected and the nucleic acid probes immobilized on the substrate, without removing molecules, which are not hybridized with the nucleic acid probes immobilized on the substrate, from the reaction chamber.

In this aspect of the present invention, it is an essential feature of a preferred embodiment of the method according to the present invention that the detection of a hybridization between the nucleic acids to be detected and the nucleic acids immobilized on the substrate of the microarray is performed without removing those molecules, which are not hybridized with the nucleic acids immobilized on the substrate, from the reaction chamber. Such molecules can, for example, be primers equipped with a detectable marker, which have not been converted during the amplification reaction, or nucleotides or nucleic acid molecules equipped with a detectable marker, for which no complementary nucleic acid probe is present on the array, which specifically hybridizes with said nucleic acid,i.e., the detection of the interaction and/or hybridization between nucleic acid targets and nucleic acid probes can be performed without requiring washing or rinsing steps subsequently to the hybridization.

Although in this aspect of the present invention, in the method according to the present invention, preferably no washing step for removing molecules, which are not hybridized with the nucleic acids immobilized on the substrate, is performed, it was surprisingly revealed that an exact and sensitive detection of the specific hybridization between target nucleic acids and nucleic acid probes immobilized on the substrate is ensured despite the background of the solution caused by said molecules, which have not been removed.

Thus, in this aspect of the present invention, the method according to the present invention allows the amplification and the qualitative and quantitative detection of nucleic acids in a reaction chamber, wherein the detection of molecular interactions or hybridizations can be performed immediately after completion of a cyclic amplification reaction, preferably without requiring an exchange of the sample or reaction liquids. Furthermore, the method according to the present invention also ensures a cyclic detection of hybridization events in the amplification, i.e., a detection of the hybridization also during the cyclic amplification reaction. Finally, with the aid of the method according to the present invention, the amplification products can be quantified during the amplification reaction as well as after completion of the amplification reaction.

Thus, in a preferred embodiment of the method according to the present invention, the detection is performed during the cyclic amplification reaction or after completion of the cyclic amplification reaction. Preferably, the detection is performed during the amplification reaction with each amplification cycle. Alternatively, the detection can also be determined with every second cycle or every third cycle or arbitrarily in other intervals, however.

Preferably, the cyclic amplification reaction is a PCR. In a PCR, three temperatures for each PCR cycle are usually passed through. Preferably, the hybridized nucleic acids detach from the microarray at the highest temperature, i.e., the denaturation temperature. A preferred value for the denaturation temperature is 95° C. Therefore, a measurement value, which serves as zero value or rather reference value for the nucleic acids detected in the respective PCR cycle, can be determined at this denaturation temperature.

At the temperature following in the PCR cycle, an annealing temperature of, for example, about 60° C., a hybridization between the nucleic acids to be detected and the nucleic acids immobilized on the substrate of the microarray is facilitated. Therefore, in one embodiment of the method according to the present invention, the detection of oligonucleotides present in a PCR cycle is performed at the annealing temperature.

In order to enhance the sensitivity of the method according to the present invention, it can further be advantageous to lower the temperature below the annealing temperature, so that the detection is preferably performed at a temperature below the annealing temperature of an amplification cycle. For example, the detection can be performed at a temperature within a range of 25° C. to 50° C. and preferably within a range of 30° C. to 40° C.

In a further alternative embodiment of the method according to the present invention, the hybridization between nucleic acids to be detected and the nucleic acids immobilized on the substrate of the microarray is at first performed at a low temperature, in order to subsequently increase the hybridization temperature. Such an embodiment has the advantage that the hybridization time is decreased compared to hybridization at temperatures of below 50° C. without losing specificity in the interactions.

If the zero value or reference value determined at denaturation temperature is subtracted from the measurement value determined at or below the annealing temperature, a measurement result free of disturbances, in which fluctuation and drift are eliminated, can be obtained.

Within the scope of the present invention, the detection of an interaction between the probe and the target molecule is usually performed as follows: After fixing the probe or the probes at a specific matrix in the form of a microarray in a given manner, the targets are contacted with the probes in a solution and are incubated under defined conditions. As a result of the incubation, a specific interaction or hybridization occurs between probe and target. The bond occurring herein is significantly more stable than the bond of target molecules to probes not specific for the target molecule.

The detection of the specific interaction between a target and its probe can be performed by means of a variety of methods, which normally depend on the type of marker, which has been inserted into target molecules before, during, or after the interaction of the target molecule with the microarray. Typically, such markers are fluorescent groups, so that specific target/probe interactions can be read out fluorescence-optically at high local resolution and, compared to other conventional detection methods, in particular mass-sensitive methods, with low effort (see for example A. Marshall, J. Hodgson, DNA chips: An array of possibilities, Nature Biotechnology 1998, 16, 27-31; G. Ramsay, DNA Chips: State of the art, Nature Biotechnology 1998, 16, 40-44).

Depending on the substance library immobilized on the microarray and the chemical nature of the target molecules, interactions between nucleic acids and nucleic acids, between proteins and proteins, and between nucleic acids and proteins can be examined by way of this test principle (for survey see F. Lottspeich, H. Zorbas, 1998, Bioanalytik, Spektrum Akademischer Verlag, Heidelberg/Berlin).

Herein, antibody libraries, receptor libraries, peptide libraries, and nucleic acid libraries can be used as substance libraries, which are immobilized on microarrays or chips.

The nucleic acid libraries play the most important role by far. They are microarrays, whereon deoxyribonucleic acid (DNA) molecules or ribonucleic acid (RNA) molecules are immobilized.

Preferably, the nucleic acids to be detected are equipped with a detectable marker. Particularly preferably, the detectable marker is a fluorescence marker. The signal of the molecules in solution, which are not hybridized with the nucleic acid probes of the array, i.e., the background, can be kept low, in comparison with the signal of the nucleic acids hybridized with the nucleic acid probes, in particular by means of using especially narrow reaction chambers, in particular in the form of a capillary gap, in the method according to the present invention. The enrichment of target molecules caused by the specific binding of probe and target allows the imaging of the signals on the microarray, for example, also by means of a fluorescence-optical system imaging the entire volume of the reaction chamber, provided that the reaction chamber is designed in a sufficiently narrow manner, preferably in the form of a capillary gap.

Thus, in a preferred embodiment of the method according to the present invention, the sample is inserted into a reaction chamber, which is designed in the form of a capillary gap between the chamber support and the microarray. Preferably, the capillary gap has a thickness in the range of 10 μm to 200 μm, particularly preferably in the range of 25 μm to 150 μm, and most preferably in the range of 50 μm to 100 μm. In special embodiments, the capillary gap has a thickness of 60 μm, 70 μm, 80 μm, or 90 μm.

In an alternative embodiment, the method according to the present invention is performed in a reaction space or a reaction chamber having, for example, a thickness of 0.7 mm to 2.5 mm, preferably of 1.0 mm to 2.0 mm, and particularly preferably of 0.8 mm to 1.8 mm. In a special embodiment, the thickness of the reaction space is 1.1 mm.

It has surprisingly shown that, in the method according to the present invention, a signal-to-noise ratio is obtained, which allows an exact and sensitive detection despite the background caused by labeled molecules in solution, which might not have been removed. The following hypothesis is assumed, without intending to be bound by it. As the concentration of the nucleic acids to be detected in solution increases, the surface of the chip or microarray is saturated. This correspondingly influences the saturation of the signal to be detected. It results from the dependency of the concentration of target molecules in solution on the thickness of the thickness of the reaction chamber that, in particular with the reaction chamber designed in the form of a capillary gap, a sensible detection of the amplification products against the background can be performed with, for example, a chamber thickness of 200 μm or less. The use of such narrow reaction chambers allows the use of a simple detection technique without requiring washing or rinsing steps subsequently to the hybridization between nucleic acid probes and target nucleic acids. Furthermore, the performance of dynamic experiments, like they are, for example, required in examinations of the melting behavior of probe/target complexes or in the performance of a real-time detection, is also facilitated.

Due to their simple structure, epifluorescent technical setups having an imaging detection system, for example on the basis of CCD, CMOS, JFIT, or scanning PMT, as well as wavelength-selected planar or point scanning illumination, for example by means of white light sources, LED, organic LED (OLED), and the like, are particularly suitable for the detection of a hybridization, in particular with the use of reaction chambers designed in the form of a capillary gap. Besides, foci-selective detection methods can of course also be used in the method according to the present invention, like for example confocal techniques or methods based on the use of a depth-selective illumination on the basis of, for example, methods based on evanescent decoupling of excitation light (TIRF) in the sample substrate based on total reflection or the use of waveguides. Such foci-selective methods are to be preferred, in particular in cases when a further exclusion of the background signals caused by the fluorescence molecules present in the liquid, i.e., not hybridized, in order to increase the sensitivity.

Thus, with the use of very narrow reaction spaces or reaction chambers, in particular in the form of a capillary gap, all conventional devices and/or methods for imaging fluorescence detection, which are described for the measurements of fluorescence signals on planar surfaces, can be used.

Examples for this are CCD-based detectors, which implement the excitation of the fluorophores in the dark field by means of incident light or transmitted light for the purpose of discriminating optical effects like dispersion and reflections (see for example C. E. Hooper et al., Quantitative Photone Imaging in the Life Sciences Using Intensified CCD Cameras, Journal of Bioluminescence and Chemoluminescence (1990), 337-344). Further alternatives for fluorescence detection systems, which can be used in the method according to the present invention, are white light setups, like for example described in WO 00/12759, WO 00/25113 and WO 96/27025; confocal systems, like for example described in U.S. Pat. No. 5,324,633, U.S. Pat. No. 6,027,880, U.S. Pat. No. 5,585,639 and WO 00/12759; confocal excitation systems based on Nipkow discs in the case of confocal imaging, as for example described in U.S. Pat. No. 5,760,950; systems based on structured excitation distribution, as for example described in WO 98/57151; highly integrated fluorescence detection systems using micro optics, like for example described in WO 99/27140; and laser scanning systems, like for example described in WO 00/12759. A general progression of fluorescence detection methods using such conventional fluorescence detection systems is, for example, described in U.S. Pat. No. 5,324,633.

The detection of the hybridization between target nucleic acids and the nucleic acids immobilized on the substrate during the cyclic amplification reaction allows a continuous detection of the signal increase on the probe array or microarray. In a further embodiment of the method according to the present invention, the initial concentration of the nucleic acids to be detected in the sample is determined by means of correlating it with the number of amplification cycles required in order to render the hybridization between the nucleic acids to be detected and the nucleic acid probes immobilized on the substrate detectable.

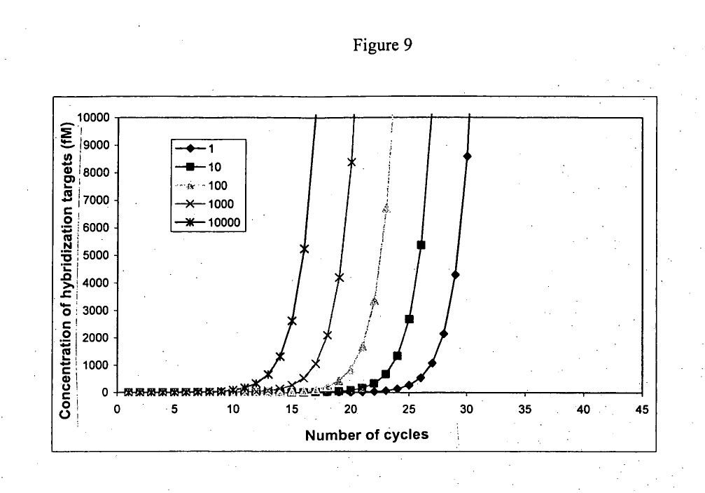

FIG. 9 shows the progression of the exponential amplification of a target with varying initial concentrations of target molecules in the sample. In performing the method according to the present invention, using conventional fluorescence detection, a typical detection limit is a target concentration in the range of 1 pM to 10 pM. FIG. 9 shows that this range is reached in dependency on the initial concentration of nucleic acids to be detected in the sample after a varying number of amplification cycles. From the number of amplification cycles required to reach this detection limit, the initial concentration of the nucleic acids to be detected in the sample can therefore be concluded.

In a particularly preferred embodiment of the method according to the present invention, the sample contains a nucleic acid, which interacts or hybridizes with a nucleic acid probe of the microarray, in known concentration. Within the scope of the present invention, such a nucleic acid of known concentration is also referred to as control nucleic acid or control.

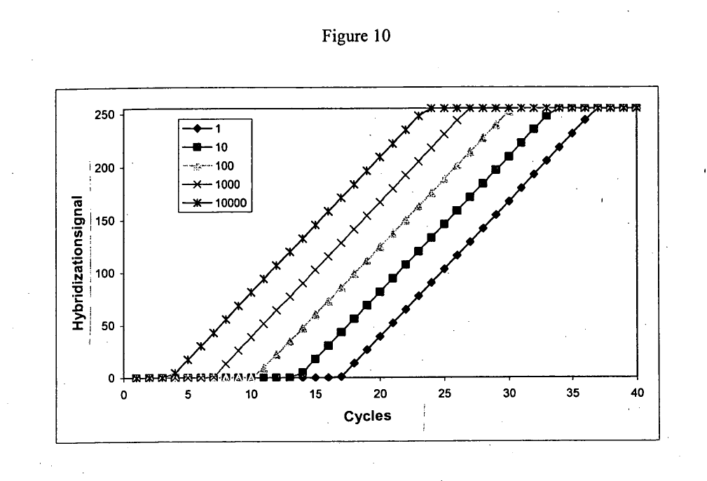

FIG. 10 shows the development of a hybridization signal in dependency on the number of amplification cycles and on the initial concentration of target nucleic acids in solution as a result of the exponential amplification. From FIG. 10 it also follows that a quantification of the target amount is possible by means of merely determining the point in time of reaching the detection limit, in particular if a corresponding control nucleic acid in known concentration is also present in the sample and a corresponding calibration is performed.

The targets to be examined can be present in every type of sample, preferably in a biological sample.

Preferably, the targets are isolated, purified, copied, and/or amplified before their detection and quantification.

Usually, the amplification is performed by means of conventional PCR methods or by means of a method for the parallel performance of amplification of the target molecules to be analyzed by means of PCR and detection by means of hybridization of the target molecules with the substance library support, as is described above.

In a further embodiment, the amplification is performed as a multiplex PCR in a two-step process (see also WO 97/45559). In a first step, a multiplex PCR is performed by way of using fusion primers, whose 3′-ends are gene specific and whose 5′-ends are universal regions. The latter are the same in all forward and reverse primers used in the multiplex reaction. In this first step, the primer amount is limiting. Hereby, all multiplex products can be amplified until a uniform molar level is achieved, given that the number of cycles is adequate for reaching primer limitation for all products. In a second step, universal primers identical to the 5′-regions of the fusion primers are present. Amplification is performed until the desired amount of DNA is obtained.

In the method according to the present invention, the detection is preferably performed in such a way that the bound targets are equipped with at least one label, which is detected in step c).

As already mentioned above, the label coupled to the targets or probes preferably is a detectable unit or a detectable unit coupled to the targets or probes via an anchor group. Concerning the possibilities for detection and/or labeling, the method according to the present invention is highly flexible. Thus, the method according to the present invention is compatible with a variety of physical, chemical, or biochemical detection methods. The only prerequisite is that the unit or structure to be detected can directly be coupled, and/or can be linked via an anchor group, which can be coupled with the oligonucleotide, to a probe or a target, for example an oligonucleotide.

The detection of the label can be based on fluorescence, magnetism, charge, mass, affinity, enzymatic activity, reactivity, a gold label, and the like. Thus, the label can, for example, be based on the use of fluorophore-labeled structures or components. In connection with the fluorescence detection, the label can be an arbitrary colorant, which can be coupled to targets or probes during or after their synthesis. Examples are Cy colorants (Amersham Pharmacia Biotech, Uppsala, Sweden), Alexa colorants, Texas Red, Fluorescein, Rhodamin (Molecular Probes, Eugene, Oreg., USA), lanthanides like samarium, ytterbium, and europium (EG&G, Wallac, Freiburg, Germany).

Besides fluorescence markers, also luminescence markers, metal markers, enzyme markers, radioactive markers, and/or polymeric markers can be used within the scope of the present invention as labeling and/or detection unit, which is coupled to the targets or the probes.

Likewise, a nucleic acid can be used as label (tag), which can be detected by means of hybridization with a labeled reporter. This is meant by “sandwich hybridization”. Diverse molecular biological detection reactions like primer extension, ligation, and RCA are used for the detection of the tag.

In an alternative embodiment of the method according to the present invention, the detectable unit is coupled with the targets or probes via an anchor group. Preferably used anchor groups are biotin, digoxigenin, and the like. In a subsequent reaction, the anchor group is converted with specifically binding components, for example streptavidin conjugates or antibody conjugates, which in turn are detectable or trigger a detectable reaction. With the use of anchor groups, the conversion of the anchor groups to detectable units can be performed before, during, or after the addition of the sample comprising the targets, or, optionally, before, during, or after the cleavage of the selectively cleavable bond in the probes.

According to the present invention, the labeling can also be performed by means of interaction of a labeled molecule with the probe molecules. For example, the labeling can be performed by means of hybridization of a labeled oligonucleotide with an oligonucleotide probe or an oligonucleotide target, as described above.

Further labeling methods and detection systems suitable within the scope of the present invention are described, for example, in Lottspeich and Zorbas, Bioanalytik, Spektrum Akademischer Verlag, Heidelberg, Berlin, 1998, chapter 23.3 and 23.4.

In a preferred embodiment of the method according to the present invention, detection methods are used, which in result yield an adduct having a particular solubility product, which leads to a precipitation. For labeling, in particular substrates or rather educts are used, which can be converted to a hardly soluble, usually stained product. In this labeling reaction, for example, enzymes can be used, which catalyze the conversion of a substrate to a hardly soluble product. Reactions suitable for leading to a precipitation at the array elements as well as possibilities for the detection of the precipitation are, for example, described in the International Patent Application WO 00/72018 and in the International Patent Application WO 02/02810, whose contents are hereby explicitly referred to.

In a particularly preferred embodiment of the method according to the present invention, the bound targets are equipped with a label catalyzing the reaction of a soluble substrate or educt to form a hardly soluble precipitation at the array element, where a probe/target interaction has occurred or rather acting as a crystal nucleus for the conversion of a soluble substrate or educts to a hardly soluble precipitation at the array element, where a probe/target interaction has occurred.

In this manner, the use of the method according to the present invention allows the simultaneous qualitative and quantitative analysis of a variety of probe/target interactions, wherein individual array elements having a size of ≦1000 μm, preferably of ≦100 μm, and particularly preferably of ≦50 μm can be implemented.

The use of enzymatic labels is known in immunocytochemistry and in immunological tests based on microwell plates (see E. Lidell and I. Weeks, Antibody Technology, BIOS Scientific Publishers Limited, 1995). Thus, for example, enzymes catalyze the conversion of a substrate to a hardly soluble, usually stained product.

Particularly preferably, the reaction leading to precipitation formation at the array elements is a conversion of a soluble substrate or educt to a hardly soluble product catalyzed by an enzyme. In a special embodiment, the reaction leading to precipitation formation at the array elements is an oxidation of 3,3′,5,5′-tetramethylbenzidine catalyzed by a peroxidase.

Horseradish peroxidase is preferably used for the oxidation of 3,3′,5,5′-tetramethylbenzidine. However, the person skilled in the art knows further peroxidases, which can be used for the oxidation of 3,3′,5,5′-tetramethylbenzidine.

It is assumed that 3,3′,5,5′-tetramethylbenzidine, under the catalytic effect of a peroxidase, is oxidized in a first step to form a blue-stained radical cation (see for example Gallati and Pracht, J. Clin. Chem. Clin. Biochem. 1985, 23, 8, 454). This blue-stained radical cation is precipitated in the form of a complex by means of a polyanion, like for example dextran sulfate. The precipitation reaction by means of peroxidase-catalyzed oxidation of 3,3′,5,5′-tetramethylbenzidine is, for example, described in EP 0 456 782.

Without claiming to be complete, the preceding Table 1 gives a survey of several reactions possibly suitable for leading to a precipitation at array elements, where an interaction between target and probe has occurred.

The labeling of biological samples with enzymes or gold, in particular nanocrystalline gold, has been sufficiently described (see, inter alia, F. Lottspeich and H. Zorbas, Bioanalytik, Spektrum Akademischer Verlag, Heidelberg, Berlin, 1998; E. Lidell and I. Weeks, Antibody Technology, BIOS Scientific Publishers Limited, 1995).

Further possibilities for the detection of the probe/target interactions via insoluble precipitates in the method according to the present invention are described in: Immunogold-Silver Staining, Principles, Methods and Applications, Hrsg.: M. A. Hayat, 1995, CRC Press; Eur J Immunogenet February-April 1991;18(1-2):33-55 HLA-DR, DQ and DP typing using PCR amplification and immobilized probes. Erlich H, Bugawan T, Begovich A B, Scharf S, Griffith R, Saiki R, Higuchi R, Walsh P S. Department of Human Genetics, Cetus Corp., Emeryville, Calif. 94608; Mol Cell Probes June 1993;7(3):199-207 A combined modified reverse dot-blot and nested PCR assay for the specific non-radioactive detection of Listeria monocytogenes. Bsat N, Batt C A. Department of Food Science, Cornell University, Ithaca, N.Y. 14853. Immunogenetics 1990;32(4):231-41 Erratum in: Immunogenetics 1991;34(6):413 Rapid HLA-DPB typing using enzymatically amplified DNA and nonradioactive sequence-specific oligonucleotide probes. Bugawan T L, Begovich A B, Erlich H A. Department of Human Genetics, Cetus Corporation, Emeryville, Calif. 94608. Hum Immunol December 1992;35(4):215-22 Generic HLA-DRB1 gene oligotyping by a nonradioactive reverse dot-blot methodology. Eliaou J F, Palmade F, Avinens O, Edouard E, Ballaguer P, Nicolas J C, Clot J. Laboratory of Immunology, Saint Eloi Hospital, CHU Montpellier, France. J Immunol Methods Nov. 30, 1984;74(2):353-60 Sensitive visualization of antigen-antibody reactions in dot and blot immune overlay assays with immunogold and immunogold/silver staining. Moeremans M, Daneels G, Van Dijck A, Langanger G, De Mey J. Histochemistry 1987;86(6):609-15 Non-radioactive in situ hybridization. A comparison of several immunocytochemical detection systems using reflection-contrast and electron microscopy. Cremers A F, Jansen in de Wal N, Wiegant J, Dirks R W, Weisbeek P, van der Ploeg M, Landegent J E.

Within the scope of the present invention, the following variants, inter alia, are conceivable for the detection of the probe/target interactions via insoluble precipitates in the methods according to the present invention.

In one embodiment of the methods according to the present invention, the targets are equipped with a catalyst, preferably an enzyme, which catalyzes the conversion of a soluble substrate or educt to an insoluble product. In this case, the reaction leading to precipitation formation at the array elements is a conversion of a soluble substrate or educt to an insoluble product in the presence of a catalyst coupled with one of the targets, preferably an enzyme. Preferably, the enzyme is selected from the group consisting of horseradish peroxidase, alkaline phosphatase, and glucose oxidase. The soluble substrate or educt is preferably selected from the group consisting of 3,3′-diaminobenzidine, 4-chloro-1-naphthol, 3-amino-9-ethylcarbazole, p-phenylenediamine-HCl/pyrocatechol, 3,3′,5,5′-tetramethylbenzidine, naphthol/pyronin, brom-chlor-indoyl-phosphate, nitroblue tetrazolium, and phenazine methosulfate. For example, a colorless soluble hydrogen donor, for example 3,3′-diaminobenzidine, is converted to an insoluble stained product in the presence of hydrogen peroxide. The enzyme horseradish peroxidase transfers hydrogen ions from the donors to hydrogen peroxide while forming water.

In a preferred embodiment of the present invention, the reaction leading to precipitation formation at the array elements is the formation of a metallic precipitation. Particularly preferably, the reaction leading to precipitation formation at the array elements is the chemical reduction of a silver compound, preferably silver nitrate, silver lactate, silver acetate, or silver tartrate, to form elemental silver. Formaldehyde and/or hydroquinone are preferably used as reducing agents.

Thus, a further possibility for the detection of molecular interaction on arrays is the use of metal labels. Herein, for example colloidal gold or defined gold clusters are coupled with the targets, optionally via particular mediator molecules like streptavidin. The staining resulting from gold labeling is preferably enhanced by the subsequent reaction with less precious metals, like for example silver, wherein the gold label coupled with the targets acts as crystal nucleus or catalyst, for example, for the reduction of silver ions to a silver precipitate. The targets coupled with gold labels are also referred to as gold conjugates in the following.

Further possibilities of the detection of probe/target interactions via insoluble precipitates are, in particular, described in WO 02/02810.

In this embodiment of the method according to the present invention, a relative quantification of the probe/target interaction can also be performed. The relative quantification of the concentration of the bound targets on a probe array by means of detection of a precipitate is performed via the concentration of the labels coupled with the targets, which catalyze the reaction of a soluble substrate to form a hardly soluble precipitate on the array element, where a probe/target interaction has occurred or which act as crystal nucleus for such reactions. For example in the case of oligonucleotide probes labeled with nanogold and purified with HPLC, the ratio of bound target to gold particles is 1:1. In other embodiments of the present invention, the ratio can be a multiplicity or also a fraction thereof.

Thus, in this embodiment of the detection method according to the present invention, the detection is performed by means of measuring the transmission alteration, reflection, or dispersion caused by the precipitate, which is generated by the catalytic effect of the label coupled with the bound targets on the array elements, where a probe/target interaction has occurred.

In the case of coupling colloidal gold or defined gold clusters with the targets, light absorption is already evoked by the presence of these metallic labels. In order to enhance the light absorption, however, a non-transparent precipitate is precipitated preferably catalytically by such interactive hybrids, i.e., targets equipped with a label like, for example, colloidal gold or defined gold clusters. In the case of gold conjugates, the use of silver as precipitate has turned out to be particularly preferable.

Thus, in a further preferred embodiment of the method according to the present invention, the time-dependent behavior of the precipitation formation at the array elements is detected in the form of signal intensities in step c). In this manner, an exact determination of the relative quantitative amount of targets bound can be ensured. Such a procedure is described in detail in the International Patent Application WO 02/02810, whose contents are hereby explicitly referred to.

In this aspect of the present invention, the qualitative and/or quantitative detection of the probe/target interaction by means of measuring the absorption of transmitted light can be performed analogously to the above-described example for a method according to the present invention using a device according to the present invention comprising an optical system, by means of which the time-dependent behavior of precipitation formations on the detection area is detectable.

It is a prerequisite for binding a target molecule, which is for example labeled with a fluorescence group, in the form of a DNA or RNA molecule to a nucleic acid of the microarray that both the target molecule and the probe molecule are present in the form of a single-stranded nucleic acid. An efficient and specific hybridization can only occur between such molecules. Single-stranded nucleic acid target and nucleic acid probe molecules are normally obtained by means of heat denaturation and optimal selection of parameters like temperature, ionic strength, and concentration of helix-destabilizing molecules. Therefore, it is guaranteed that only probes having sequences of almost perfect complementarity, i.e., closely matching one another, remain paired with the target sequence (A. A. Leitch, T. Schwarzacher, D. Jackson, I. J. Leitch, 1994, In vitro Hybridisierung, Spektrum Akade-mischer Verlag, Heidelberg/Berlin/Oxford).

In a further preferred embodiment, if the nucleic acid to be detected is at first amplified by means of a PCR, at least one competitor inhibiting the formation of one of the template strands amplified by means of the PCR is added to the reaction at the beginning. In the PCR, in particular a DNA molecule is added, which competes against one of the primers used for the PCR amplification of the template for binding to the template and which cannot be enzymatically extended. The single-stranded nucleic acid molecules amplified by means of the PCR are then detected by means of hybridization with a complementary probe. Alternatively, the nucleic acid to be detected is first amplified in single strand surplus by means of a PCR and is then detected by means of a subsequent hybridization with a complementary probe, wherein a competitor, which is a DNA molecule or a molecule of a nucleic acid analog capable of hybridizing to one of the two strands of the template but not to the region detected by means of probe hybridization and which cannot be extended enzymatically, is added to the PCR reaction at the beginning.

Every molecule causing a preferred amplification of only one of the two template strands present in the PCR reaction can be used as competitor in the PCR. According to the present invention, competitors can be proteins, peptides, DNA ligands, intercalators, nucleic acids, or analogs thereof. According to the present invention, proteins or peptides, which are capable of binding single-stranded nucleic acids with sequence specificity and which have the above-defined properties, are preferably used as competitors. Particularly preferably, nucleic acid molecules and nucleic acid analog molecules are used as secondary structure breakers.

The formation of one of the two template strands is substantially inhibited by initial addition of the competitor to the PCR during the amplification. “Substantially inhibited” means that within the scope of the PCR a single strand surplus and an amount of the other template strand are produced, which suffice to allow an efficient detection of the amplified strand by means of the hybridization. Therefore, the amplification does not follow exponential kinetics of the form 2ⁿ (with n=number of cycles), but rather attenuated amplification kinetics of the form <2ⁿ.

The single strand surplus obtained by means of the PCR in relation to the non-amplified strand has the factor 1.1 to 1,000, preferably the factor 1.1 to 300, also preferably the factor 1.1 to 100, particularly preferably the factor 1.5 to 100, also particularly preferably the factor 1.5 to 50, in particular preferably the factor 1.5 to 20, and most preferably the factor 1.5 to 10.

Typically, the function of a competitor will be to bind selectively to one of the two template strands and therefore to inhibit the amplification of the corresponding complementary strand. Therefore, competitors can be single-stranded DNA- or RNA-binding proteins having specificity for one of the two template strands to be amplified in a PCR. They can also be aptamers sequence-specifically binding only to specific regions of one of the two template strands to be amplified.

Nucleic acids or nucleic acid analogs are preferably used as competitors in the method according to the present invention. Conventionally, the nucleic acids or nucleic acid analogs will act as competitors of the PCR by either competing against one of the primers used for the PCR for the primer binding site or by being capable of hybridizing with a region of a template strand to be detected due to a sequence complementarity. This region is not the sequence detected by the probe. Such nucleic acid competitors are enzymatically not extendable.

The nucleic acid analogs can be e.g., so-called peptide nucleic acids (PNA). However, nucleic acid analogs can also be nucleic acid molecules, in which the nucleotides are linked to one another via a phosphothioate bond instead of a phosphate bond. They can also be nucleic acid analogs, wherein the naturally occurring sugar components ribose or deoxyribose have been replaced with alternative sugars like e.g., arabinose or trehalose. Furthermore, the nucleic acid derivative can be “locked nucleic acid” (LNA). Further conventional nucleic acid analogs are known to the person skilled in the art.

DNA or RNA molecules, in particular preferably DNA or RNA oligonucleotides or analogs thereof, are preferably used as competitors.

Depending on the sequence of the nucleic acid molecules or nucleic acid analogs used as competitors, the inhibition of the amplification of one of the two template strands within the scope of the PCR reaction is based on different mechanisms. By way of the example of a DNA molecule, this is discussed in the following.

If, for example, a DNA molecule is used as competitor, it can have a sequence, which is at least partially identical to the sequence of one of the primers used for the PCR in such a way that a specific hybridization of the DNA competitor molecule with the corresponding template strand is possible under stringent conditions. As, according to the present invention, the DNA molecule used for competition in this case is not extendable by means of a DNA polymerase, the DNA molecule competes for binding to the template against the respective primer during the PCR reaction. According to the ratio of the DNA competitor molecule and the primer, the amplification of the template strand defined by the primer can thus be inhibited in such a way that the production of this template strand is significantly reduced. Herein, the PCR proceeds according to exponential kinetics higher than would be expected with respect to the amounts of competitors used. In this manner, a single strand surplus emerges in an amount, which is sufficient for the efficient detection of the amplified target molecules by means of hybridization.

In this embodiment, the nucleic acid molecules or nucleic acid analogs used for competition must not be enzymatically extendable. “Enzymatically not extendable” means that the DNA or RNA polymerase used for the amplification cannot use the nucleic acid competitor as primer, i.e., it is not capable of synthesizing the corresponding opposite strand of the template 3′ from the sequence defined by the competitor.

Alternatively to the above-depicted possibility, the DNA competitor molecule can also have a sequence complementary to a region of the template strand to be detected, which is not addressed by one of the primer sequences and which is enzymatically not extendable. Within the scope of the PCR, the DNA competitor molecule will then hybridize to this template strand and correspondingly block the amplification of this strand.

The person skilled in the art knows that the sequences of DNA competitor molecules or generally nucleic acid competitor molecules can be selected correspondingly. If the nucleic acid competitor molecules have a sequence, which is not substantially identical to the sequence of one of the primers used for the PCR, but is complementary to another region of the template strand to be detected, this sequence is to be selected in such a way that it does not fall within the region of the template sequence, which is detected with a probe within the scope of the hybridization. This is necessary because there does not have to occur a processing reaction between the PCR and the hybridization reaction. If a nucleic acid molecule, which falls within the region to be detected, were used as competitor, it would compete for binding to the probe against the single-stranded target molecule.

Such competitors preferably hybridize near the template sequence detected by the probe. Herein, according to the present invention, the position specification “near” is to be understood in the same way as given for secondary structure breakers. However, the competitors according to the present invention can also hybridize in the immediate proximity of the sequence to be detected, i.e., in exactly one nucleotide's distance from the target sequence to be detected.

If enzymatically not extendable nucleic acids or nucleic acid analogs are used as competing molecules, they are to be selected according to their sequence and structure in such a way that they cannot be enzymatically extended by DNA or RNA polymerases. Preferably, the 3′-end of a nucleic acid competitor is designed in such a way that it has no complementarity to the template and/or has at its 3′-end another substituent instead of the 3-OH group.

If the 3′ end of the nucleic acid competitor has no complementarity to the template, regardless of whether the nucleic acid competitor binds to one of the primer binding sites of the template or to one of the sequences of the template to be amplified by means of the PCR, the nucleic acid competitor cannot be extended by the conventional DNA polymerases due to the lack of base complementarity at its 3′-end. This type of non-extensibility of nucleic acid competitors by DNA polymerases is known to the person skilled in the art. Preferably, the nucleic acid competitor has no complementarity to its target sequence at its 3′-end with respect to the last 4 bases, particularly preferably to the last 3 bases, in particular preferably to the last 2 bases and most preferably to the last base. In the mentioned positions, such competitors can also have non-natural bases, which do not allow hybridization.

Nucleic acid competitors, which are enzymatically not extendable, can also have a 100%-complementarity to their target sequence, if they are modified in their backbone or at their 3′-end in such a way that they are enzymatically not extendable.

If the nucleic acid competitor has at its 3′-end a group other than the OH group, these substituents are preferably a phosphate group, a hydrogen atom (dideoxynucleotide), a biotin group, or an amino group. These groups cannot be extended by the conventional polymerases.

The use of a DNA molecule, which competes for binding to the template against one of the two primers used for the PCR and which was provided with an amino link at its 3′-end during chemical synthesis, as a competitor in such a method is particularly preferred. Such competitors can have a 100% complementary to their target sequence.

However, nucleic acid analogue competitors, like for example PNAs do not need to have a blocked 3′ OH group or a non-complementary base at their 3′-end as they are not recognized by the DNA polymerases because of the backbone modified by the peptide bond and thus they are not extended. Other corresponding modifications of the phosphate group, which are not recognized by the DNA polymerases, are known to the person skilled in the art. Belonging thereto are, inter alia, nucleic acids having backbone modifications, like for example 2′-5′ amide bonds (Chan et al. (1999) J. Chem. Soc., Perkin Trans. 1, 315-320), sulfide bonds (Kawai et al. (1993) Nucleic Acids Res., 1 (6), 1473-1479), LNA (Sorensen et al. (2002) J. Am. Chem. Soc., 124 (10), 2164-2176) and TNA (Schoning et al. (2000) Science, 290 (5495), 1347-1351).

Several competitors hybridizing to different regions of the template (for example the primer binding site, inter alia) can also simultaneously be used in a PCR. The efficiency of the hybridization can additionally be increased, if the competitors have properties of secondary structure breakers.

In an alternative embodiment, the DNA competitor molecule can also have a sequence complementary to one of the primers. Depending on the ratio of antisense DNA competitor molecule and primer, such for example antisense DNA competitor molecules can then be used to titrate the primer in the PCR reaction, so that it will no longer hybridize with the corresponding template strand and, correspondingly, only the template strand defined by the other primer is amplified. The person skilled in the art is aware of the fact that, in this embodiment of the invention, the nucleic acid competitor can, but does not need to, be enzymatically extendable.

If, within the scope of the present invention, it is talked about nucleic acid competitors, this includes nucleic acid analog competitors, unless a different meaning arises from the respective context. The nucleic acid competitor can bind to the corresponding strand of the template reversibly or irreversibly. The binding can take place by means of covalent or non-covalent interactions.

Preferably, binding of the nucleic acid competitor takes place via non-covalent interactions and is reversible. In particular preferably, binding to the template takes place via formation of Watson-Crick base pairings.

The sequences of the nucleic acid competitors normally adapt to the sequence of the template strand to be detected. In the case of antisense primers, though, they adapt to the primer sequences to be titrated, which are in turn defined by the template sequences, however.

PCR amplification of nucleic acids is a standard laboratory method, whose various possibilities of variation and development are familiar to the person skilled in the art. In principle, a PCR is characterized in that the double-stranded nucleic acid template, usually a double-stranded DNA molecule, is first subjected to heat denaturation for 5 minutes at 95° C., whereby the two strands are separated from each other. After cooling down to the so-called “annealing” temperature (defined by the primer with the lower melting temperature), the forward and reverse primers present in the reaction solution accumulate at those sites in the respective template strands, which are complementary to their own sequences. Herein, the “annealing” temperature of the primers adapts to the length and base structure of the primers. It can be calculated on the basis of theoretical considerations. Information on the calculation of “annealing” temperatures can be found, for example, in Sambrook et al. (vide supra).

Annealing of the primers, which typically is performed within a range of temperature between 40 to 75° C., preferably between 45 to 72° C. and in particular preferably between 50 to 72° C., is followed by an elongation step, wherein deoxyribonucleotides are linked with the 3′-end of the primers by the activity of the DNA polymerase present in the reaction solution. Herein, the identity of the inserted dNTPs depends on the sequence of the template strand hybridized with the primer. As normally thermostable DNA polymerases are used, the elongation step usually runs at between 68 to 72° C.

In a symmetrical PCR, an exponential increase of the nucleic acid region of the target defined by the primer sequences is achieved by means of repeating this described cycle of denaturation, annealing and elongation of the primers. With respect to the buffer conditions of the PCR, the usable DNA polymerases, the production of double-stranded DNA templates, the design of primers, the selection of the annealing temperature, and variations of the classic PCR, the person skilled in the art has numerous works of literature at his disposal.

It is familiar to the person skilled in the art that also, for example, single-stranded RNA, like for example mRNA, can be used as template. Usually, it is previously transcribed into a double-stranded cDNA by means of a reverse transcription.

In a preferred embodiment, a thermostable DNA-dependent polymerase is used as polymerase. In a particularly preferred embodiment, a thermostable DNA-dependent DNA polymerase is used, which is selected from the group consisting of Taq-DNA polymerase (Eppendorf, Hamburg, Germany and Qiagen, Hilden, Germany), Pfu-DNA polymerase (Stratagene, La Jolla, USA), Tth-DNA polymerase (Biozym Epicenter Technol., Madison, USA), Vent-DNA polymerase, DeepVent-DNA polymerase (New England Biolabs, Beverly, USA), Expand-DNA polymerase (Roche, Mannheim, Germany).

The use of polymerases, which have been optimized from naturally occurring polymerases by means of specific or evolutive alteration, is also preferred. When performing the PCR in the presence of the substance library support, the use of the Taq-polymerase by Eppendorf (Germany) or of the Advantage cDNA Polymerase Mix by Clontech (Palo Alto, Calif., USA) is in particular preferred.

In another aspect of the present invention a device for the amplification and for the qualitative and quantitative detection of nucleic acids by means of a method according to the present invention, as described above, is provided.

In this aspect of the present invention, the device comprises a temperature controlling and/or regulating unit; a reaction chamber formed between a chamber support and a microarray, wherein the microarray comprises a substrate with nucleic acid probes immobilized on array elements thereon and wherein the temperature in the reaction chamber can be controlled and/or regulated by means of the temperature controlling and regulating unit. The device is developed in such a manner, that a hybridization between the nucleic acids can be detected and the nucleic acid probes immobilized on the substrate can be detected by means of the device without removing those molecules from the reaction chamber, which are not hybridized with the nucleic acids immobilized on the substrate.

A chip or microarray inside the reaction chamber, wherein the chip or microarray comprises a support with a detection area, whereon a substance library is immobilized, ensures the possibility of providing a very high probe density in the reaction chamber.

In this aspect, the reaction chamber of the device according to the present invention is preferably developed in the form of a capillary gap. The capillary gap preferably has a thickness within a range of 10 μm to 200 μm, particularly preferably within a range of 25 μm to 150 μm and most preferably within a range of 50 μm to 100 μm. In special embodiments, the capillary gap has a thickness of 60 μm, 70 μm, 80 μm, or 90 μm.

In an alternative embodiment of the device according to the present invention, the reaction space or the reaction chamber has, for example, a thickness of 0.7 mm to 2.5 mm, preferably of 1.0 mm to 2.0 mm, and particularly preferably of 0.8 mm to 1.8 mm. In a special embodiment, the thickness of the reaction space is 1.1 mm.

The electrocaloric control and/or regulation by means of the temperature controlling and/or regulating unit allows the setting of defined temperatures both during processing of the sample to be examined in the reaction chamber and during the detection of the hybridization events. Thus, both an improved control and an optimization of the detection reaction are ensured. Furthermore, the setting of defined temperatures by means of the temperature controlling and/or regulating unit allows the performance of complex reactions, like for example of amplification reactions by means of PCR.

In general, the devices according to the present invention allow a performance of processing and/or conditioning reactions, which is almost simultaneous, time-efficient and exhibits a low fault liability as well as the chip-based characterization of nucleic acids. Herein, according to the present invention, a processing and/or conditioning reaction is understood to denote a reaction, whose reaction products can be characterized by means of chip-based experiments.

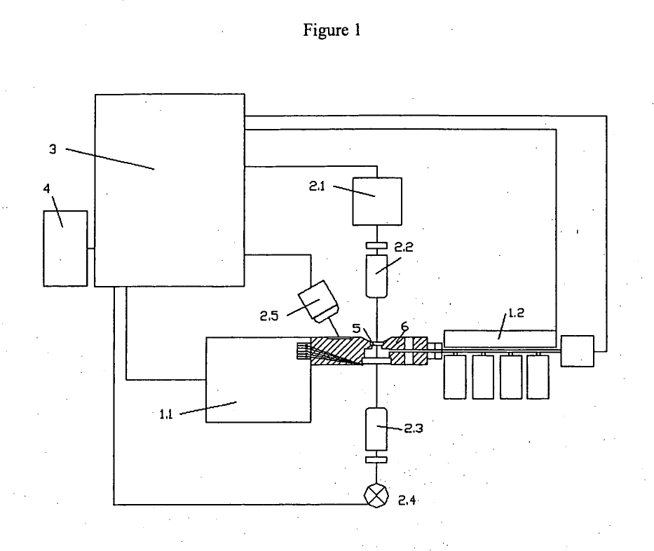

A device according to the present invention for the detection of molecular interactions in closed reaction chambers preferably consists of four principal functional elements (see FIG. 1). The mechanical, electrical, and fluidic recording of the reaction chamber is performed in a recording module (1). In the following, the reaction chamber is also referred to as microreactor. For the detection of the reaction results, an optical system (2) is provided. The processing of the reaction results to an analysis result can be performed in a controller (3). Optionally, the analysis result is made available for storage and/or further processing by means of suitable connecting elements (4).

A reaction chamber, which can be used as component of the device according to the present invention in an advantageous manner, is described in detail in the International Patent Application WO 01/02094, whose contents are hereby explicitly referred to.

The reaction chamber, which can optionally be identified by means of a bar code, is integrated in a fluidic recording module, where it can be filled with one or more reaction solutions. Optionally, the reaction chamber further has electrical contacts, whereby a thermal control and/or regulation of reactions in the reaction chamber, for example by means of integrated sensor and/or heating elements, is ensured. In particular, this is advantageous for the performance of thermally sensitive amplification reactions for DNA or RNA, hybridizations of DNA or RNA, or reactions for the enhancement of signals, like for example by means of metal precipitations at target molecules, which are correspondingly labeled and bound to the substance library.

The solutions optionally required for the performance of the amplification and detection reactions, like reaction and/or rinsing solutions, can be inserted into the reaction chamber via suitable connecting elements, like for example channels. Suitable controllers can be used for the supervision of the course of the reaction.

The devices according to the present invention further ensure the transfer of the raw data or analysis results to external computers or computer networks, for example for storage of said data, via optionally existing electronic interfaces.

Preferably, the device comprises a detection system, in particular an optical system, particularly preferably a fluorescence optical system.

In a further preferred embodiment, the fluorescence-optical system is a system imaging the entire volume of the reaction chamber. Compared to confocal fluorescence systems or systems for the detection by means of evanescent field excitation (see for example Biosensors and Bioelectronics, 18 (2003) 489-497), such systems have the advantage of being more easily to operate and more cost-efficient.

Examples for fluorescence-optical systems imaging the entire volume of the reaction chamber are epifluorescent technical setups having an imaging detection system, for example on the basis of CCD, CMOS, JFIT, or scanning PMT, as well as wavelength-selected plane or point scanning illumination, for example by means of white light sources, LED, organic LED (OLED), and the like.

Besides, foci-selective detection methods can of course also be used in the method according to the present invention, like for example confocal techniques or methods based on the use of a depth-selective illumination on the basis of, for example, methods based on evanescent decoupling of excitation light (TIRF) in the sample substrate based on total reflection or the use of waveguides. Such foci-selective methods are to be preferred, in particular in cases when a further exclusion of the background signals caused by the fluorescence molecules present in the liquid, i.e., not hybridized, in order to increase the sensitivity.

Thus, with the use of very narrow reaction spaces or reaction chambers, in particular in the form of a capillary gap, all conventional devices and/or methods for imaging fluorescence detection, which are described for the measurements of fluorescence signals on planar surfaces, can be used.

In another preferred embodiment of the device according to the present invention comprises a device for the amplification and for detection of nucleic acids, a optical system, preferably a fluorescent optical system or a optical system developed in such a way, that the time-dependent behavior of the alteration of transmission properties of the detection area is detectable.

In this aspect of the present invention, the device according to the present invention is characterized in that a detection of molecular interaction is also possible in manual operation due to the integrated optical system or reader system. This is particularly advantageous in fields like medical diagnostics. An exact determination of the relative quantitative amount of nucleic acids bound to the substance library is ensured by the fact that, in this embodiment, the device according to the present invention contains an integrated optical system, by means of which the time-dependent behavior of precipitation formations on the detection area is detectable.

The optical system ensures imaging of the substance library during or after completion of the amplification and/or detection reactions on a suitable detector, which is for example implemented in the form of a two-dimensional electrically readable detection element. In one embodiment of the device according to the present invention, the sample is illuminated by means of an illumination module or a light source of the optical system and the emerging signals are imaged in a filtered manner correspondingly to the labels used.

Furthermore, the optical system ensures a kinetic, i.e., dynamic, recording of the reaction results. In particular, the optical system of the device according to the present invention is suitable for recording the time-dependent behavior of a silver precipitation for the enhancement of hybridization signals between gold-labeled target molecules and the substance library. The highly integrated setup of the device according to the present invention allows the transfer of several images during the course of the reaction for processing in a suitable data processing module or controller.

The alterations originating from the time-dependent behavior of precipitation formation on the respective array elements can be evaluated in the device according to the present invention as is described in detail, inter alia, in the International Patent Application WO 02/02810. The respective contents of the International Patent Application WO 02/02810 are hereby also explicitly referred to.

The optical system, by means of which the time-dependent behavior of precipitation formations on the detection area of the chip is detectable, preferably comprises a two-dimensionally readable detector. Preferably, the detector is a camera, in particular a CCD or CMOS camera or a similar camera. The use of cameras having electric image converters, like for example CCD or CMOS chips, allows the realization of high local resolutions.

The cameras used in the optical system of the device according to the present invention ensure that the illumination intensity is dispersed homogenously on the area to be imaged and that the signals to be detected can be imaged by means of reflection, transmission modulation, dispersion, polarization modulation and the like by means of the applied detection technique within the scope of the available dynamics. Such illumination methods are described, for example, in the International Patent Application WO 00/72018 and are commercially available (for example by Vision & Control GmbH (Suhl, Germany) for dark field illuminations and by Edmund Industrieoptik GmbH (Karlsruhe, Germany) for LED circular light).

A high local resolution of the area to be detected can, for example, also be achieved by imaging on detectors like mirror arrays or LCD elements and their adjustment according to a pattern to be detected or an area to be defined, as is for example described for fluorescence uses in the German laid-open patent application DE 199 14 279. The advantage of such a detector in measurement of reflection or transmission modulations is the integration of thermal, electric, and fluidic control and/or regulation, the possibility of optical signal processing and thus the lower technical demands made on the computer technology involved.

Normally, the detectors record the entire area of the probe array.

Alternatively, scanning detectors can also be used for reading out the chip. In the use of scanning point light sources and/or scanning detectors, the device according to the present invention comprises movable optical components for the direction of light and/or movable mechanical components for the attachment of the reaction chambers, so that directing the respective components across the individual positions to be scanned, i.e., the respective measurement points, is ensured. In this embodiment, image recording is performed by means of computational reconstruction of the image from the respective measurement points. In particular, the camera in this embodiment is a movable line camera.

Furthermore, the optical system preferably comprises in addition a light source, particularly preferably a multispectral or a coherent light source. Examples for light sources within the scope of the present invention are lasers, light emitting diodes (LED), and/or high pressure lamps. The light source of the optical system preferably ensures a homogenous illumination of the support.

In addition to point light sources, light sources in the form of illumination arrays can also be used in the device according to the present invention. In this implementation, a homogenous illumination of the support can, for example, also be ensured by the light source comprising several diffusely radiating light sources, whose overlay results in a homogenous illumination. Thus, for example diffusely dispersing LEDs, which are aligned in the form of a matrix, allow a homogenous illumination at short distances from the sample.

As already mentioned above, the device according to the present invention can be implemented in such a way that the detection area can be scanned in lines by the light source. If a raster-like or scanning direction of the light beam across the detection area is desired, the following embodiments of the device according to the present invention are conceivable:

For example, the detection area and/or the reaction chamber can be implemented in a movable manner and can be directed past a stationary light source. If the light source is a laser, the laser is in inoperative position herein. Furthermore, the detection area can be in inoperative position and a movable laser beam can be directed across the detection area. Finally, it is also possible that the light source is moved in an axis and the detection area is moved in another axis.

In another advantageous embodiment of the device according to the present invention, the device additionally comprises lenses, mirrors, and/or filters. The use of filters on the one hand allows spectral limitation of the homogenous illumination and on the other hand illumination of the samples with different wavelengths. In another variant, the device according to the present invention comprises filter changers. By means of said filter changers, the optical filters can be changed quickly and therefore possibly incorrect information, which for example occurs due to impurities, can be recognized unambiguously and can be eliminated.

As already mentioned above, the optical system is preferably developed in such a way that the detection area can be illuminated homogenously, preferably with an illumination intensity homogeneity of at least 50%, particularly preferably of at least 60% and most preferably of at least 70%.

In another preferred embodiment of the device according to the present invention, the optical system is developed in such a way that the time-dependent behavior of the alteration of transmission properties of the detection area is detectable. This can, for example, be ensured by light source and detector being arranged on opposite sides inside the reaction chamber and the reaction chamber including the support for the detection area being optically transparent at least in the region of the optical path leading from the light source to the detector.

In a further embodiment, the optical system is arranged in such a way that the time-dependent behavior of the alteration of reflection properties of the detection area is detectable. In a preferred embodiment for measuring the reflectivity, a surface mirror is added on the lower side of the support element. In this embodiment, the disadvantage of the poor reflection of the sample is supplemented by transmission effects, wherein the illumination light reflects via a mirror layer behind the sample, either in the form of an autonomous mirror or in the form of a layer applied to the back side of the sample support.

Herein, for example, a planar emitter can be arranged on the side opposite of the support element and therefore also towards the sensor of, for example, a CCD camera. In this manner, a very compact layout is rendered possible. In a further preferred embodiment, in particular if the reflectivity of the support element is measured, the device additionally comprises a semi-transparent mirror between light source and support element. In this embodiment, the light of the light source reaches the sample through a semi-transparent mirror and the image is imaged on a camera in reflection through the semi-transparent mirror and, optionally, through an optical read-out system.

In a particularly preferred embodiment of the device according to the present invention, the optical system is arranged in such a way that the time-dependent behavior of the alteration of dispersion properties of the detection area is detectable. Herein, light source and detector are preferably arranged on the same side of the area to be detected. In this embodiment, the optical system can, for example, be arranged in such a way that the sample and/or the chip can be illuminated in a particular angle, which preferably is smaller than 45° and particularly preferably smaller than 30°. The illumination angle is selected in such a way that the irradiated light, in the absence of local dispersion centers, i.e., before a precipitation formation on the detection area, is not directly reflected into the optical detection path and therefore no signal is detectable. If local dispersion centers occur on the detection area, for example due to formation of a precipitation, a part of the irradiated light reaches the optical detection path and therefore leads to a measurable signal in the optical system of the device according to the present invention.

Particularly preferably, the chamber support or the substance library support is optically not transparent in this embodiment, at least in the region of the detection area. Suitable optically not transparent materials are, for example, silicon, ceramic materials, or metals. The use of an optically not transparent chamber support has the advantage that, due to advantageous physical properties of the support materials, an easier, more exact and more homogenous temperature control of the reaction chamber is ensured, so that a successful performance of temperature-sensitive reactions like a PCR is guaranteed.

In a further preferred embodiment of the device according to the present invention, the optical excitation path of the light source is designed in such a way that regions of parallel light are present and therefore interference filters can be inserted into the optical system without displacing their transmission windows.

In a further preferred embodiment of the device according to the present invention, the optical detection path is designed in such a way that regions of parallel light are present, and therefore interference filters can be inserted into the optical system without displacing their transmission windows.

In non-parallel optical paths, interference filters strongly alter their spectral selectivity. If the optical system, due to the presence of regions of parallel light, allows the insertion or arrangement of interference filters in the device according to the present invention without altering their spectral selectivity, the device according to the present invention is also suitable for the detection of molecular interactions of substances labeled with fluorochromes. Thus, a universal CCD-based reaction and detection device can be implemented.

In this embodiment of the device according to the present invention, which is also suitable for the detection of fluorescence labeling, the optical system, with the use of white or multispectral light like, for example, halogen illumination, xenon, white light LED and the like, can for example have two filters in the optical illumination and detection path or, with the use of monochrome light sources like, for example, LED or laser, it can have, for example, one filter in the optical detection path.

For example, the device according to this aspect of the present invention can be implemented in such a manner that the chamber body of the reaction chamber containing the chip with the detection area is sealingly applied to a chamber support in such a way that a sample space having a capillary gap between the chamber support and the detection area or rather the substrate of the chip is formed, whose temperature is adjustable and whose volume flow rate is controllable. This type of construction allows the performance of reactions, which only run efficiently within a particular range of temperature, and the preferably simultaneous detection of the reaction products by means of chip-based experiments.

Thus, the device according to the present invention can, for example, be used for amplifying the nucleic acid molecules by means of PCR and almost simultaneously detecting the PCR products by means of chip-based experiments. The sample liquid for such reactions can be efficiently heated or cooled by corresponding means for temperature regulation.

The device according to the present invention can also be used for performing a reverse transcriptase reaction and thereby transferring mRNA to cDNA and characterizing the reaction products by means of hybridization to the chip. In this manner, a so-called “gene profiling” can be performed. As both the reverse transcription and the hybridization are performed inside a chamber, this method is highly time-efficient and exhibits a low fault liability.

With the device according to the present invention, for example, a restriction digestion at desired temperatures can furthermore be performed inside the reaction chamber and the reaction products can be characterized by means of hybridization to a chip. Denaturation of the enzymes can be performed by means of heat deactivation. Thus, the device according to the present invention allows a time-efficient restriction-fragment-length-polymorphism mapping (RFLP mapping).

Furthermore, with the device according to the present invention, for example a ligation can also be performed.

With the device according to the present invention, the temperature-dependent melting behavior of nucleic acid target/nucleic acid probe complexes can furthermore be examined.

Furthermore, devices according to the present invention can be used for performing the temperature-dependent binding behavior of proteins. In this manner, it can for example be tested if antibodies are still capable of binding their respective antigens after a long period of heating. In this case, it is a prerequisite that the chip is not functionalized by nucleic acid molecules, but by the respective proteins or peptides.

The chamber body of the reaction chamber preferably consists of materials like glass, synthetic material and/or metals like high-grade steel, aluminum, and brass. For its manufacturing, for example synthetic materials suitable for injection molding can be used. Inter alia, synthetic materials like macrolon, nylon, PMMA, and teflon are conceivable. Alternatively, the reaction space between substance library support and chamber support can be closed by means of septa, which for example allow filling of the reaction space by means of syringes. In a preferred embodiment, the chamber body consists of optically transparent materials like glass, PMMA, polycarbonate, polystyrene, and/or topaz. Herein, the selection of materials is to be adjusted to the intended use of the device. For example, the temperatures the device will be exposed to are to be considered when selecting the materials. If, for example, the device shall be used for performing a PCR, for example only synthetic materials may be used, which remain stable for longer periods at temperatures like 95° C.

The chamber support preferably consists of glass, synthetic materials, silicon, metals, and/or ceramic materials. The chamber support can, for example, consist of aluminum oxide ceramics, nylon, and/or teflon.

In one embodiment, the chamber support consists of transparent materials like glass and/or optically transparent synthetic materials, for example PMMA, polycarbonate, polystyrene, or acrylic.

Preferably, the chamber support and/or the substrate is connected with means for temperature increase, which are integrated into the device according to the present invention, and should then preferably consist of materials having high thermal conductivity. Such thermally conductive materials offer the substantial advantage of ensuring a homogenous temperature profile covering the entire area of the reaction space and therefore temperature-dependent reactions like, for example, a PCR can be performed homogenously, with high yield, and controllably and/or regulably at great exactitude in the entire reaction chamber.

Thus, in a preferred embodiment, the chamber support and/or the substrate consist of materials having a high thermal conductivity, preferably a thermal conductivity in the range of 15 to 500 Wm⁻¹K⁻¹, particularly preferably in the range of 50 to 300 Wm⁻¹K⁻¹ and most preferably in the range of 100 to 200 Wm⁻¹K⁻¹, wherein the materials are usually not optically transparent. Examples for suitable thermally conductive materials are silicon, ceramic materials like aluminum oxide ceramics, and/or metals like high-grade steel, aluminum, or brass.

In a particularly preferred embodiment, the substrate consists of materials having a high thermal conductivity, like for example ceramic materials. In this embodiment, the substrate is connected with a means for temperature increase, whereby the opposite side, the chamber support, can be made of a material not having a distinct thermal conductivity, like for example a material, which is also used for the remaining chamber body. Thus, as opposed to an embodiment wherein both the chamber support and the substrate are made of a cost-intensive material, a cost-intensive component is eliminated in this embodiment.

If the substrate or the support of the device according to the present invention substantially consist of ceramic materials, aluminum oxide ceramics are preferably used. Examples for such aluminum oxide ceramics are the ceramics A-473, A-476, and A-493 by Kyocera (Neuss, Germany). The ceramics substantially differ in their respective aluminum oxide content (A-473: 93%, A-476: 96%, and A-493: 99%) as well as in their surface roughness. Aluminum oxide ceramics having a surface roughness as low as possible are most preferably used.

Preferably, the chamber support and/or the substrate is equipped on its reverse side, i.e., the side facing away from the reaction chamber, with optionally miniaturized temperature sensors and/or electrodes or rather has heating structures in this place, so that tempering of the sample liquid as well as mixing of the sample liquid by means of an induced electro-osmotic flow is possible.

The temperature sensors can, for example, be implemented in the form of nickel-chromium thin film resistance temperature sensors.

The electrodes can, for example, be implemented in the form of gold-titanium electrodes and, in particular, in the form of a quadrupole.

The means for temperature increase can preferably be selected in such a way that fast heating and cooling of the liquid in the capillary gap is possible. Herein, fast heating and cooling is understood to signify that temperature alterations in a range of 0.2 K/s to 30 K/s, preferably of 0.3 K/s to 15 K/s, particularly preferably of 0.5 K/s to 12 K/s and most preferably of 2 K/s to 10 K/s can be mediated by the means for temperature increase. Preferably, temperature alterations of 5 K/s to 11 K/s can also be mediated by the means for temperature increase.

The means for temperature increase, for example in the form of heaters, can also be implemented in the form of nickel-chromium thin film resistance heaters, for example.

For further details on the specification and dimension of the temperature sensors, means for temperature increase, and the electrodes, it is referred to the contents of the International Patent Application WO 01/02094.

The chip or rather the substrate can preferably consist of borofloat glasses, silica glass, single-crystal CaF₂, sapphire discs, topaz, PMMA, polycarbonate, and/or polystyrene. The selection of materials is also to be adjusted according to the intended use of the device and/or the chip. If, for example, the chip is used for the characterization of PCR products, only materials, which can resist a temperature of 95° C., may be used.

Preferably, the chips are functionalized by nucleic acid molecules, in particular by DNA or RNA molecules. However, they can also be functionalized by peptides and/or proteins, like for example antibodies, receptor molecules, pharmaceutically active peptides, and/or hormones.

If the detection of the time-dependent behavior of precipitation formations on the detection area is performed in the dark field, i.e., if alterations of the dispersion properties of the detection area are detected, suitable materials for the substance library support are optically transparent materials like glass, particularly preferably borosilicate glass, and transparent polymers, like for example PMMA, polycarbonate, and/or acrylic; suitable materials for the chamber support are optically transparent materials like glass and/or synthetic materials and, in particular, optically not transparent materials like silicon, ceramic materials; suitable materials for the reaction chamber are synthetic materials like macrolon, PMMA, polycarbonate, teflon and the like, metals like high-grade steel, aluminum, and/or brass as well as glass. In the performance of dark field measurements of the device according to the present invention, the chamber support can alternatively consist of optically transparent materials, while the substance library support consists of optically not transparent materials.

In one embodiment, the device according to the present invention additionally comprises at least one fluid container, which is connected with the reaction chamber, and optionally a unit for controlling the loading and unloading of the reaction chamber with fluids. Within the scope of the present invention, fluids are understood to denote liquids and gases. The connection of the fluid containers with the reaction chamber can, for example, be implemented as is described in the International Patent Application WO 01/02094.

In a further embodiment, the device according to the present invention comprises a unit, which is connected with the optical system, for processing the signals recorded by the optical system. This coupling of detection unit and processing unit, which ensures the conversion of the reaction results into the analysis result, allows, inter alia, the use of the device according to the present invention as hand-held unit, for example in medical diagnostics.

Preferably, the device furthermore comprises an interface for external computers. This allows the transfer of data for storage purposes outside the device.

In a further preferred embodiment, the reaction chamber is individually marked via a data matrix. To this end, when manufacturing the device according to the present invention, a data record containing information on the substance library, the performance of the detection reaction, and the like is stored in a database. Thus, the data record can in particular contain information on the layout of the probes on the array as well as information on how the evaluation is to be conducted in the most advantageous manner. The data record or the data matrix can further contain information on the temperature-time regime of a PCR, which is optionally to be performed for the amplification of the target molecules. The data record compiled in that manner is preferably equipped with a number, which is attached to the support in the form of the data matrix. By means of the number registered in the data matrix, the compiled data record can then optionally be accessed when reading out the substance library. Finally, the data matrix can be read out by the temperature controlling and/or regulating unit and by other controllers, like for example a control for loading and unloading of the reaction chamber, via the fluid containers and thus an automatic performance of amplification and detection reactions can be ensured.

In a further aspect of the present invention, a device for the amplification and detection of nucleic acids is provided, which also contains a temperature controlling and/or regulating unit as described above as well as a reaction chamber as described above comprising a support having a detection area, whereon a substance library is immobilized, wherein the temperature in the reaction chamber can be controlled and/or regulated by means of the temperature controlling and/or regulating unit.

In this aspect of the present invention, the device has electric contacts at the respective array spots instead of an optical system, however. These electric contacts can be contacted, for example, via electrodes. Due to the formation of a metallic precipitation on the array elements for signal enhancement of the, for example, gold-labeled targets bound to the substance library, a conductive material epitaxially grows at those array spots, at which such a binding has occurred, which leads to an alteration of the local resistance. Thus, a modulation of particular electric parameters, like for example conductivity, resistance, and permeability, is possible via the electric contacts at the array spots.

Preferably, in this aspect of the present invention, the substance library support of the device according to the present invention has a three-dimensional structure, which is, for example, formed by bumps, base and/or through holes, whereby the effect in the melting of the epitaxially growing conductive material is supported by the forming precipitate with the electric contacts and the alteration of electric parameters resulting therefrom.

The device according to the present invention based on optical detection preferably has a fluidics unit for the exchange of solutions in the reaction chamber, a temperature controlling and/or regulating unit as well as an optical system suitable for dynamic measurements. In a device-related sense, the above-mentioned units can optionally also be developed separately by means of implementation of corresponding interfaces.

In particular, substance libraries immobilized on the microarrays or chips are protein libraries like antibody, receptor protein, or membrane protein libraries, peptide libraries like receptor ligand libraries, libraries of pharmacologically active peptides or libraries of peptide hormones, and nucleic acid libraries like DNA or RNA molecule libraries. Particularly preferably, they are nucleic acid libraries.

As already mentioned above, the substance library preferably is immobilized on the substance library support or the detection area in the form of a microarray, particularly preferably having a density of 2 to 10,000 array spots per cm², most preferably having a density of 50 to 5,000 array spots per cm².

Furthermore, in all of the above-described embodiments of the device according to the present invention, a pre-amplification of the material to be analyzed is not required. From the sample material extracted from bacteria, blood, or other cells, specific partitions can be amplified and hybridized to the support with the aid of a PCR (polymerase chain reaction), in particular in the presence of the device according to the present invention or the substance library support as described in DE 102 53 966. This signifies a substantial reduction of labor expenditure.

Thus, the device according to the present invention is particularly suitable for the use in parallel performance of amplification of the target molecules to be analyzed by means of PCR and the detection by means of hybridization of the target molecules with the substance library support.

In another independent aspect of the present invention, a microarray comprising a substrate or a support, whereon molecular probes are immobilized on predetermined regions, is provided, wherein the substrate or the support essentially comprises ceramic materials.

Within the scope of the present invention, a support element, or support, or substance library support, or substrate is understood to denote a solid body, on which the probe array is set up.

The entirety of molecules laid out in array layout on the substrate or on the detection area, or of the substance library laid out in array layout on the substrate or on the detection area, and of the support or substrate is also referred to as microarray or probe array.

In particular, substance libraries immobilized on the substrates according to the present invention are protein libraries like antibody, receptor protein, or membrane protein libraries, peptide libraries like receptor ligand libraries, libraries of pharmacologically active peptides or libraries of peptide hormones, and nucleic acid libraries like DNA or RNA molecule libraries. Particularly preferably, they are nucleic acid libraries.

As already mentioned above, the substance library preferably is immobilized on the substrate or the substance library support or the detection area in the form of a microarray, particularly preferably having a density of 2 to 10,000 array spots or array elements per cm², most preferably having a density of 50 to 5,000 array spots or array elements per cm².

A substrate preferably essentially consisting of ceramic materials has the considerable advantage that such substrates exhibit a high thermal conductivity and thus, in the performance of temperature-dependent reactions like for example a PCR, ensure a homogenous temperature profile covering the entire area of the reaction space, which is usually limited at one side by the substrate, whereon the substance library is arranged. Thus, temperature-dependent reactions can be performed with high yield and controllably and/or regulably at high exactitude. Herein, it is preferred that the substrate of the microarray is connected with means for increasing the temperature, as have already been described in the above in connection with the devices according to the present invention.

Thus, the substrate of the microarray according to the present invention preferably essentially consists of ceramic materials having a high thermal conductivity, preferably a thermal conductivity in the range of 15 to 500 Wm⁻¹K⁻¹, particularly preferably in the range of 50 to 300 Wm⁻¹K⁻¹ and most preferably in the range of 100 to 200 Wm⁻¹K⁻¹

Within the scope of the present invention, ceramic materials are in particular materials which have been manufactured by means of annealing or firing of fine-particle, mostly wet, molded clays at temperatures of, for example, 1,000 to 1,500° C.

Preferably, the ceramic materials comprise one component, i.e., one ceramic material. In alternative embodiments, however, blends of ceramic materials, which can for example be used as laminates, are also conceivable.

Preferably, the substrate essentially comprises one or more aluminum oxide ceramics. In a particularly preferred embodiment, at least 90%, preferably at least 95%, and most preferably at least 99,5% of the substrate consists of one or more aluminum oxide ceramics. Examples for such aluminum oxide ceramics are the ceramics A-473, A-476, and A-493 by Kyocera (Neuss, Germany).

Further preferably, the substrate has a surface roughness of 0.04 μm to 0.12 μm, preferably of 0.06 μm to 0.1 μm and particularly preferably of about 0.08 μm.

Furthermore, depending on the construction of the device, which the microarray is fit into, as well as depending on the detection method used, it can be preferred that the substrate is optically transparent.

On the one hand, the transparency of the substrate can be ensured by means of the substrate having a correspondingly low thickness, regardless of the material.

On the other hand, optically transparent materials like glass ceramics can be used. The use of glass ceramics not exhibiting too great a difference regarding their indices of refraction between glass and crystal phase is preferred. An example for an optically transparent material is Ceran® (Schott, Germany). The use of lithium-alumosilicate glass ceramics is also possible.

In a further preferred embodiment, the molecular probes are immobilized on the substrate surface via a polymeric linker, for example a modified silane layer. Such a polymeric linker can serve for the derivative preparation of the substrate surface and therefore for the immobilization of the molecular probe. In the case of covalent binding of the probes, polymers, for example silanes, are used, which have been functionalized and/or modified by means of reactive functionalities like epoxides or aldehydes. Furthermore, the person skilled in the art is also familiar with the activation of a surface by means of isothiocyanate, succinimide and imido esters. To this end, amino-functionalized surfaces are often correspondingly derivatized. Furthermore, the addition of coupling reagents, like for example dicyclohexylcarbodiimide, can ensure corresponding immobilizations of the molecular probes.

In particular, the molecular probes are selected from antibodies, protein receptors, peptides, and nucleic acids.

For the setup of the microarray, it is referred to the above-mentioned description in connection with the devices according to the present invention.

In a further aspect of the present invention, a method is provided for producing a microarray according to the present invention, as is described above, comprising immobilizing molecular probes on predetermined regions of the substrate surface of a substrate essentially consisting of ceramic materials.

Preferably, the substrate essentially consists of aluminum oxide ceramics.

In a further preferred embodiment of the method according to the present invention for producing a microarray, the substrate surface is coated with a polymeric linker and the molecular probes are immobilized on the substrate surface via the polymeric linker. Particularly preferably, the polymeric linker is a modified silane layer.

The microarray according to the present invention can be used in arbitrary methods for the qualitative and/or quantitative detection of target molecules in a sample by means of molecular interaction between target molecules and the molecular probes on the microarray. In particular, the array according to the present invention is used for examining the genotypic and/or physiological state of cells.

Furthermore, the microarray according to the present invention can be used for amplifying and/or detecting nucleic acids, in particular in the above-described methods according to the present invention.

A further aspect of the present invention relates to the use of a substrate for producing a microarray, whereon molecular probes are immobilized on predetermined regions, wherein the substrate essentially comprises ceramic materials. In this aspect of the present invention, the ceramic materials can be implemented as mentioned above within the scope of the description of the microarray according to the present invention.

Of course, embodiments described for particular aspects of the present invention are also possible alternatives for corresponding other aspects of the present invention, even if it is not explicitly referred thereto.

In the following, particularly preferable embodiments of the present invention are described:

FIG. 1 schematically shows a preferred configuration of a device according to the present invention. A reaction chamber (6) or a microreactor (6) having a substance library (5) laid out on a detection area is suitably fixed in the device. The chamber (6) is electrically and fluidically connected with a temperature-processing unit (1.1) and a fluid-processing unit (1.2), so that a control and/or regulation of the temperature and/or the exchange of liquids or gases between chamber (6) and the containers of the fluid processing unit (1.2) can be implemented. The reaction chamber (6) is assigned to a process progression defined in a process controller (3) by means of an identification system (2.5), like for example a barcode reader. Thus, the respective parameters can be transferred to the processing units (1.1) and (1.2) for the processing of a test. Depending on the test-specific requirements, the optical system can be activated by means of the process controller (3) during or after the fluidic and/or thermal processing, so that a preferably dynamic optical detection process can be performed.

In the embodiment shown in FIG. 1, the detection is performed by means of detecting the alteration of the transmission properties of the sample regions, in a transmitted light system by means of a light source (2.4), which can be illuminated on the sample by means of an optical illumination system (2.3) with an intensity dispersion homogeneity of preferably at least 30% and can be imaged on a suitable detector (2.1), like for example a camera, by means of an optical detection system (2.2). The images generated in this manner can be digitalized in the detection system or in the process controller (3) and can also be analyzed in the latter. Alternatively, the images generated can also be transferred to external computers or computer networks via a data interface (4).

The modular structure of these components allows the processing of different tests with different temperature, fluid direction, and detection parameters. By means of a connection with external databases via the data interface (4), new process management parameter protocols can be implemented at any time. If specific tests, particularly in the field of medical diagnostics, shall be performed without an external data connection, the interface (4) is preferably not accessible to the user, so that all process management protocols required as well as the necessary analyses are implemented in the process controller (3).

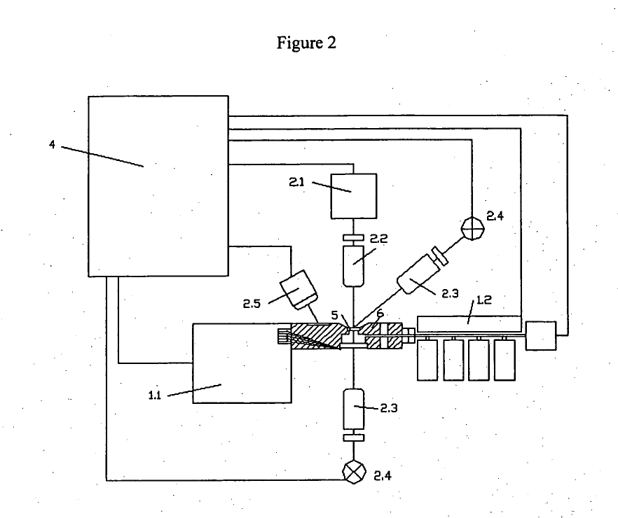

In an alternative embodiment of the device according to the present invention, which is shown in FIG. 2, process management can be performed via an external computer. The distribution of data flows to the individual technical units or modules of the device according to the present invention can be implemented via the interface (4). In this embodiment, optionally different illumination systems (2.3) and (2.4) are furthermore shown, by means of which the detection area can be illuminated from below and diagonally from above. Thus, in this embodiment, different detection methods can be activated, depending on the type of the chip determined by means of a detector for identification (2.5), for example a data matrix reader. In this embodiment, for example, the time-dependent behavior of the alteration of optical transmission properties as well as of the alteration of dispersion properties, which can be induced in the dark field in incident light, can be detected.

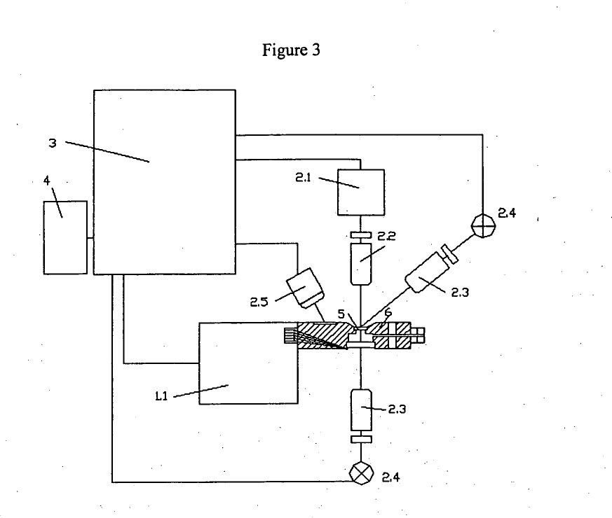

In a further embodiment of the present invention, the device according to the present invention has no fluid processing unit (see FIG. 3). This embodiment is advantageous, if it is not required for particular tests to exchange liquids during processing and if filling of the reaction space can also be performed before inserting the reaction chamber (6) into the device according to the present invention in an external manual or automated filling station.

The following examples serve the purpose of illustrating the invention and are not to be interpreted restrictively.

### EXAMPLES

In the following examples, it is essentially dealt with the detection methods, which can be performed by means of the device according to the present invention. The control of thermal parameters and the exchange of liquids can be technically implemented by means of modules, which are known to the person skilled in the art and which are also commercially available. In the technical implementation, in particular activation and data transfer, which can be performed in particular by means of weak-current connections like TTL levels as well as by means of described transfer protocols, is of particular interest herein.

### Example 1

Optical detection of kinetically proceeding signal enhancement reactions

In the following variants of the example, the optical detection of kinetically proceeding signal enhancement reactions is performed by means of recording the modulations of particular optical parameters, in particular of transmission, reflection, dispersion, diffraction, and interference.

The accumulation of silver during a silver precipitation reaction at, for example, target molecules labeled with gold particles after a specific hybridization with sample molecules immobilized on solid surfaces or synthesized sample molecules grows epitaxial layers, which significantly alter the optical properties of the entire system. The signal enhancement of gold-labeled target molecules is, for example, described in detail in the International Patent Application WO 02/02810, the contents of which are hereby explicitly referred to.

a) Transmission

The detection of the alteration of transmission properties is performed in a transmitted-light arrangement, as is shown in FIG. 1. Herein, the light source (2.4) is implemented as a white light source, like for example a halogen lamp, white light LED, or as a narrow-band light source, like for example LED, laser diode, organic LED. The optical system (2.3) consisting of lenses, mirrors, and filters is implemented in such a way that a uniform illumination of the substrate area to be detected is performed with an illumination intensity homogeneity of at least 70%.

The substrate area is imaged on a detector by means of a further optical system (2.2) consisting of lenses, mirrors, and filters. The detector can be a two-dimensional CCD or CMOS camera or a moving line camera.

The alteration of transmission properties is recorded in the form of a sequence and the curves representing the decrease in transmission are compared at different positions. Depending on the increase of transmission alteration at the respective array spots, the target concentrations can be determined quantitatively, as is described in the International Patent Application WO 02/02810.

The reaction chamber is made of a substrate transparent for the wavelengths used for the detection. For example, with the use of light within the visible range of wavelengths, the reaction chamber is made of glass, and with the use of light within the infrared range, it is made of silicon.

b) Reflection

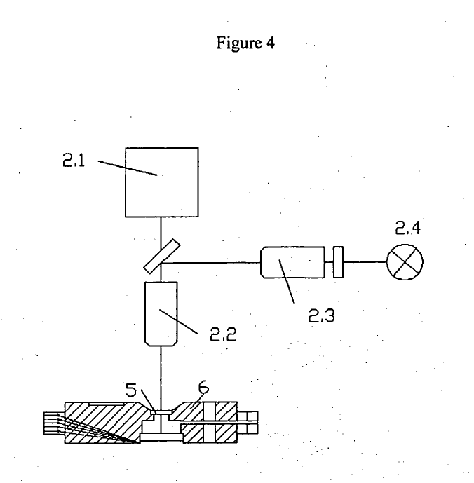

In this example, a detection of the alteration of reflection properties is performed in an incident-light arrangement, as is shown in FIG. 4. Herein, the optical system (2.3) is implemented in such a way that a homogenous illumination of the substrate surface to be detected is ensured with an illumination intensity homogeneity of at least 70%. The substrate surface is imaged on a detector by means of the optical system (2.2). The detector is a two-dimensional CCD or CMOS camera or a moving line camera.

The alteration of reflection properties is recorded in the form of a sequence and the curves representing the increase in reflection are compared at different positions depending on the increase of reflection alteration at the respective array spots, the target concentrations can be determined quantitatively, as is described in the International Patent Application WO 02/02810. Calculation is done inversely to the calculation of the detection of transmission alteration, as local reflectivity increases in the case of enrichment of silver particles at target molecules.

The reaction chamber is made of a substrate transparent for the wavelengths used for the detection. For example, with the use of light within the visible range of wavelengths, the reaction chamber is made of glass, and with the use of light within the infrared range, it is made of silicon.

c) Dispersion

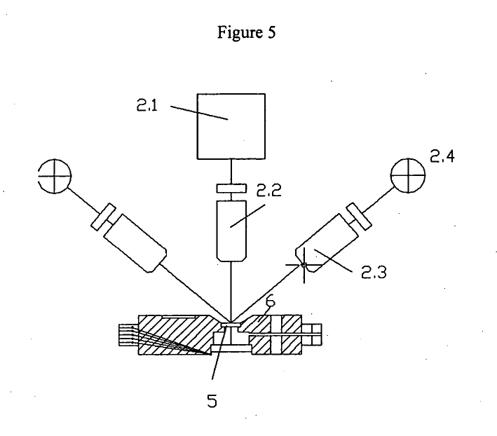

In this example, a detection of the alteration of dispersion properties is performed in a dark-field arrangement, as is shown in FIG. 5. The optical system (2.3) consisting of lenses, mirrors, and filters is implemented in such a way that a homogenous illumination of the substrate surface to be detected is performed with an illumination intensity homogeneity of at least 60%, preferably at least 70%. The substrate area is imaged on a detector by means of the optical system (2.2). The detector can be a two-dimensional camera (for example CCD, CMOS) or a moving line camera.

The alteration of dispersion properties is recorded in the form of a sequence and the curves representing the increase in dispersion are compared at different positions. Depending on the increase of reflection alteration at the respective array spots, the target concentrations can be determined quantitatively, as is described in the International Patent Application WO 02/02810. Calculation is done inversely to the calculation of the detection of transmission alteration, as the number of local dispersion centers and therefore dispersion increases in the case of enrichment of silver particles at target molecules.

When evaluating the increase of sample regions that are correspondingly classified, an exact qualitative and quantitative detection of the hybridized target molecules can be performed, preferably with a homogenous temperature distribution inside the reaction chamber.

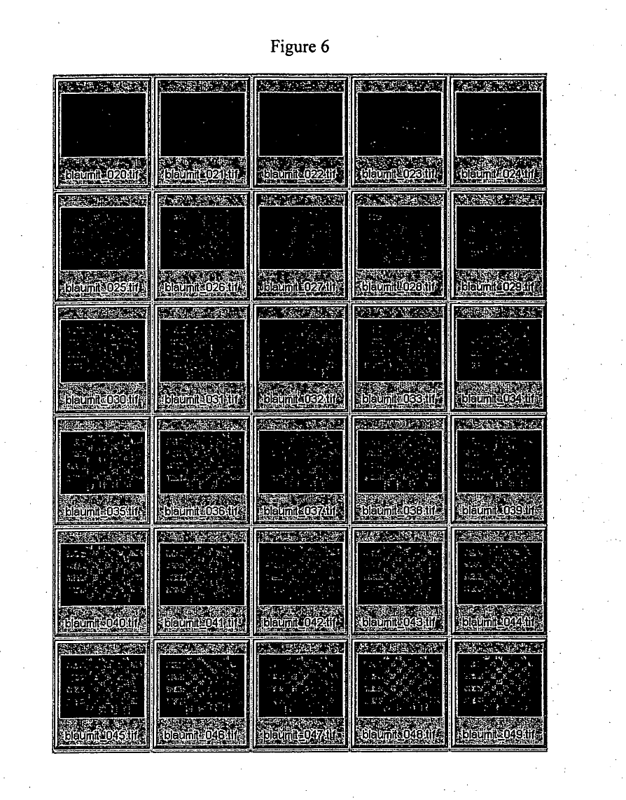

FIG. 6 shows an image sequence typical for the detection of the silver epitaxy reaction after the hybridization of specific gold-labeled target molecules on a DNA-based substance library.

The reaction chamber is made of a substrate transparent for the wavelengths used for the detection. For example, with the use of light within the visible range of wavelengths, the reaction chamber is made of glass, and with the use of light within the infrared range, it is made of silicon.

### Example 2

Electrical detection of kinetically proceeding signal enhancement reactions

In the following variants of the example, the electrical detection of kinetically proceeding signal enhancement reactions is performed by means of recording modulations of specific electrical parameters, in particular of conductivity, resistance alterations, and permeability.

The accumulation of silver during a silver precipitation reaction at, for example, target molecules labeled with gold particles after a specific hybridization with sample molecules immobilized on solid surfaces or synthesized sample molecules grows epitaxial layers, which significantly alter the optical properties of the total system. The signal enhancement of gold-labeled target molecules is, for example, described in detail in the International Patent Application WO 02/02810, the contents of which are hereby explicitly referred to.

In the device described in this example of the present invention, the individual array spots can be contacted electrically and the signals generated there can be dissipated to a measuring device in a parallel manner.

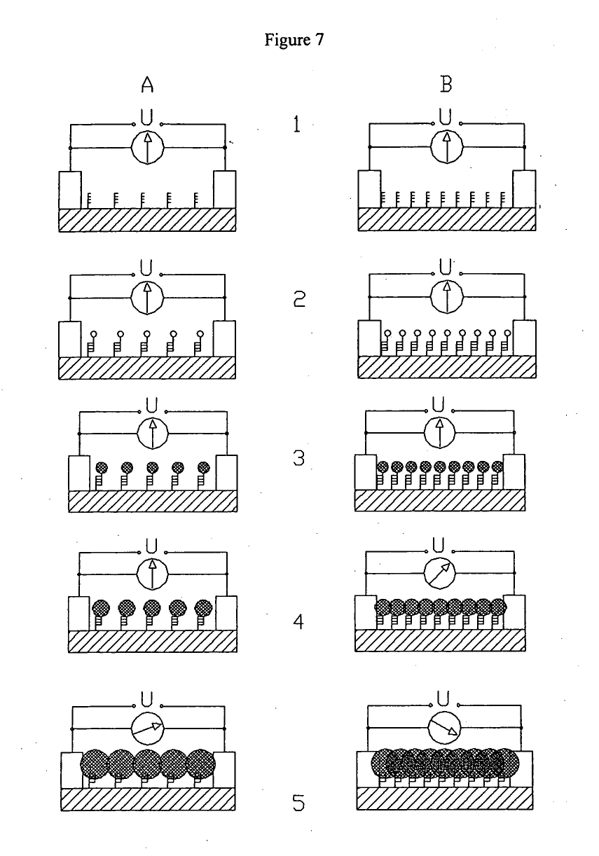

For measuring the resistance alteration at the respective array spots, the array spots are contacted electrically via electrodes, as is shown in FIG. 7. An alteration of the local resistance can be measured due to the fusion with epitaxially growing conductive material, like for example silver. Three-dimensional structures on the detection area, like for example bumps, base and through holes, support the fusion effect and the resistance alteration resulting therefrom.

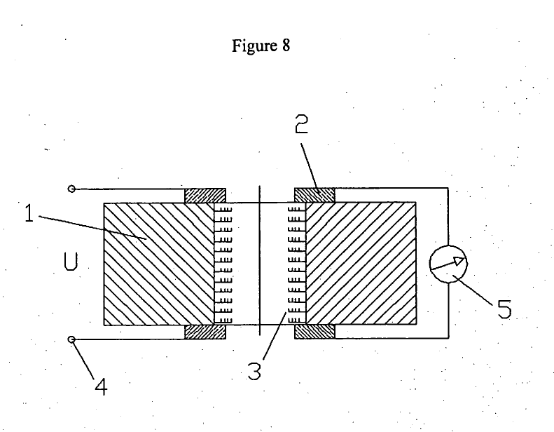

FIG. 8 schematically shows a layout for measuring said resistance alterations at one individual three-dimensional array spot.

### Example 3

Amplification and detection of nucleic acids in narrow reaction chambers

One possibility of keeping the signal in the solution low compared to the signal on or within the surface is the use of particularly narrow reaction chambers. The enrichment of target molecules on the array surface caused by the specific binding of probe and target facilitates imaging the signals on the probe array also by means of a conventional fluorescence-optical system, which illuminates and/or images the entire volume, provided that the reaction chamber is designed in a correspondingly narrow manner.

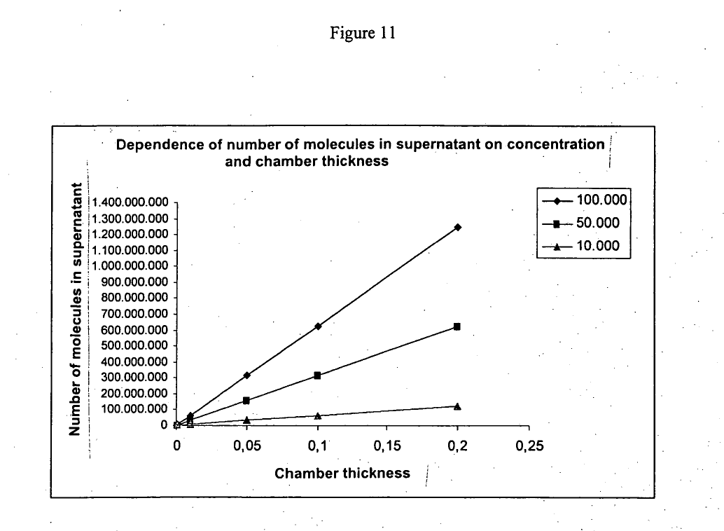

FIG. 11 shows the correlation between layer thickness and/or chamber thickness and the number of molecules labeled with a fluorescence marker, which are located in the supernatant immediately above the spot.

It has been investigated that, using a detector with 8-bit resolution, like for example an epifluorescence microscope (Zeiss, Jena, Germany), the microarrays used within the scope of the present invention exhibit signal development characteristics as depicted in the following table.

| Number of | Signal | Concentration of |
|---|---|---|
| molecules/spot | intensity | solution (pM) |
| 1,000,000,000 | 255 | 10,000,000 |
| 1,000,000,000 | 255 | 1,000,000 |
| 833,333,333 | 212.5 | 100,000 |
| 666,666,667 | 170 | 10,000 |
| 500,000,000 | 127.5 | 1,000 |
| 333,333,333 | 85 | 100 |
| 166,666,667 | 42.5 | 10 |
| 0 | 0 | 1 |
| 0 | 0 | 0.1 |

In the case of a saturated surface, the number of bound molecules present on a spot or array element of typical size can be assumed to be about 10⁹, which corresponds to a space of 10 nm² required for one molecule.

A solution containing fluorescence-labeled target (Cy3) was filled into a device according to the present invention having a reaction chamber thickness of 100 μm and a suitable probe array. The target concentration was 10 nM; 2×SSPE with 0.1% SDS was used as suitable buffer. After incubation for five minutes at 30° C., a fluorescence image was recorded. Herein, an epifluorescence microscope (Zeiss, Jena, Germany) was used, namely an Axioscope equipped with a PCO-CCD camera and having a mercury vapor lamp for fluorescence excitation. The temperature was successively increased in steps from 5° C. up to 95° C. After each increment, there also was a five-minute incubation period and an image was recorded. The image data were evaluated by means of the software Iconoclust® (Clondiag).

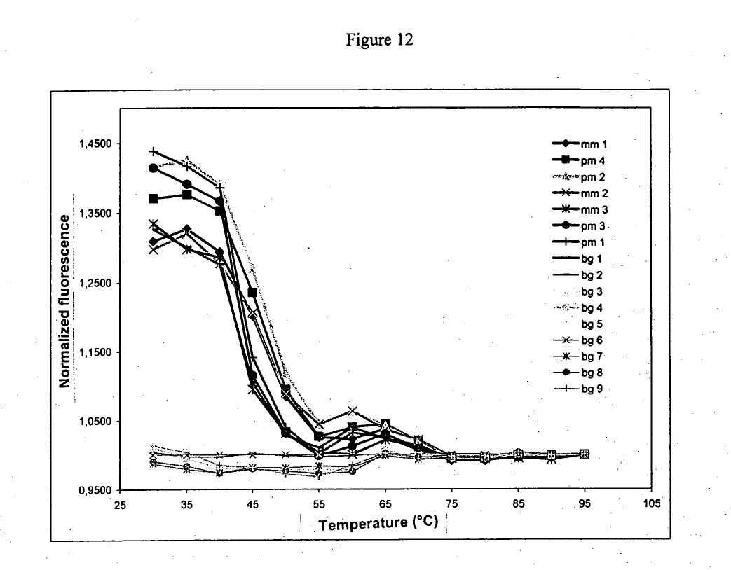

After subtraction of temperature-dependent effects, the data are illustrated in FIG. 12. FIG. 12 shows one characteristic melting curve for each of the different probes. Furthermore, the specific process of duplex dissociation in dependency of temperature and the respective sequence becomes clear.

### Example 4

Detection of nucleic acids during the amplification reaction by means of cyclic detection of hybridization events

As the amplification in the device according to the present invention is preferably performed with the aid of exponential amplification techniques like PCR, it is required for the quantification of the amount of target contained in the sample to facilitate a continuous detection of signal increase on the probe array.

The same assay as described in Example 3 was performed.

Genomic DNA from E. coli in two different dilutions was used as target material to be amplified. After a specific number of cycles, images were recorded in the manner described in Example 3. These images underwent analysis by means of the software Iconoclust® (Clondiag, Jena).

The results are illustrated in FIG. 10. The temporal delay of the amplification in the two different dilutions used is clearly evident. Likewise, the difference between match and mismatch signals is clearly evident.

### Example 5

Hybridization and subsequent detection of the hybridization by means of enzymatic precipitation of an organic molecule on a ceramic surface

Mutations of at least 6 genes of the human genome, which were identified as risk factors for the development of thromboses, have been described in the literature.

The company Ogham Diagnostics (Munster, Germany) has developed a diagnostic assay for the detection of thrombosis-relevant mutations in human DNA. With the help of an oligonucleotide-probe model system used for this test, principle detection of a hybridization and the subsequent detection by means of enzymatic precipitation of an organic colorant on a ceramic surface was to be rendered.

a) Preparation of the probe array

All of the oligonucleotides used for the preparation of the array and for the hybridization in this example have kindly been provided by Ogham Diagnostics (Münster, Germany).

25 oligonucleotides (probes) were laid out at defined positions, the so-called array elements, and covalently immobilized on an epoxidized ceramic surface (A-493, KYOCERA Fineceramics GmbH, Neuss, Germany) having an object support size of 75 mm×25 mm by means of a MicroGrid-II-arrayer (BioRobotics). The probes divide into pairs, wherein, in each case, the first probe represents the wild type and the second represents the mutation. In addition, the array was equipped with marks and control probes.

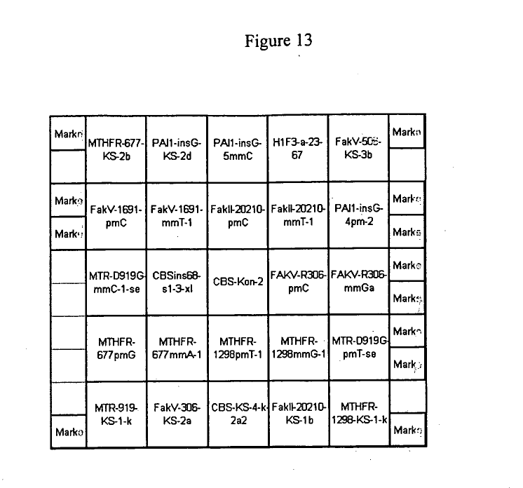

One individual complete (rectangular) probe array on the surface of the object support consisted of 10×10=100 laid-out probes and 12 marks altogether. Herein, each of the 25 oligonucleotide probes was laid out on the probe array in fourfold repetition. The array layout is shown in FIG. 13. The probes were at distances of 0.18 mm; the entire probe array covered an area of 2.16 mm×1.8 mm.

The probes were laid out on the object supports from a 10 μM solution of the oligonucleotides in 0.1 M phosphate buffer/2.2% sodium sulfate in each case. Subsequently the probes were covalently linked with the epoxide groups on the ceramic surface by means of a 30-minute period of baking at 60° C. Then followed a multisectional washing process performed in the following order:
5 min in 600 ml H₂O bidest+600 μl Triton ×100
2×2 min in 600 ml H₂O bidest+60 μl HCL (konz.)
30 min in 100 mM KCl solution
1 min rinsing in H₂O bidest
drying by means of compressed air

b) Hybridization and conjugation of the probe arrays in ArrayTube® (Clondiag)

The oligonucleotides provided for the hybridization by Ogham in the form of a mixture of six different oligonucleotides were taken up in a final concentration of 100 pM and in a final volume of 50 μl in 6×SSPE buffer (52.59 g NaCl, 8.28 g NaH₂PO₄×H₂O, 2.22 g EDTA×2H₂O in 1 l H₂O bidest, adjusted to pH 7.4 with NaOH)/0,005% Triton.

The probe array was covered with a Hybri-slip (Z36.590-4, Sigma, Taufkirchen, Germany) so that a reaction space having a volume of about 20 μl was formed above the probe array.

After denaturation of the hybridization solution (5 min 95° C.), the solution was filled into the reaction space above the probe array and subsequently the probe array was incubated for 60 minutes at 50° C. while being slightly shaken.

The Hybri-slip was then withdrawn from the ceramic surface. Two washing steps of 5 min at room temperature in 2×SSC/0.01% Triton and 2×SSC followed subsequently. Then, 200 μl of a prepared blocking solution (milk powder, Ogham, Munster, Germany in 6×SSPE/0.005% Triton) were put on the probe array; the probe array was covered with a glass cover and subsequently incubated for 10 min at room temperature. Subsequently, the glass cover was removed and the blocking solution was rinsed with 500 μl of a prepared conjugation solution (Streptavidin-poly HRP, N200, Pierce, dilution 1:10,000 in 6×SSPE/0.005% Triton). Again, 200 μl of the conjugation solution were put on the probe array; the probe array was again covered with a glass cover and incubated for 15 min at room temperature. After this period, the glass cover was removed and the array was washed for 5 min with RT in SSC/0.01% Triton, 2×SSC and 0.2×SSC in each case while being slightly shaken and subsequently dried in airflow.

c) Enzymatic precipitation, detection and evaluation

For detecting the hybridization, 100 μl of a HRP substrate solution (TrueBlue, 71-00-64, KPL, Gaithersburg, USA; HRP=horseradish peroxidase) were put on the probe array; the array was covered with a glass cover and subsequently incubated for 5 min at RT. Then, the glass cover was cautiously removed, the array was rinsed with bidest and dried in airflow.

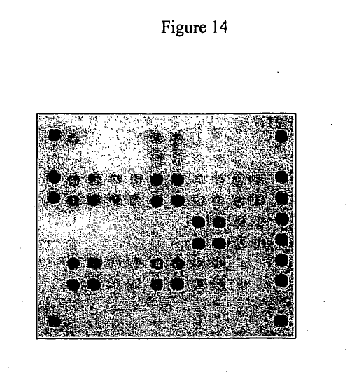

The detection was performed in transmitted light by means of a microscope (Axioskop 2, Zeiss, Jena, Germany). A picture of the stained array is shown in FIG. 14.

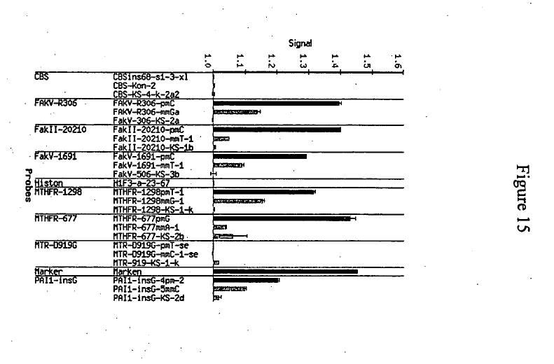

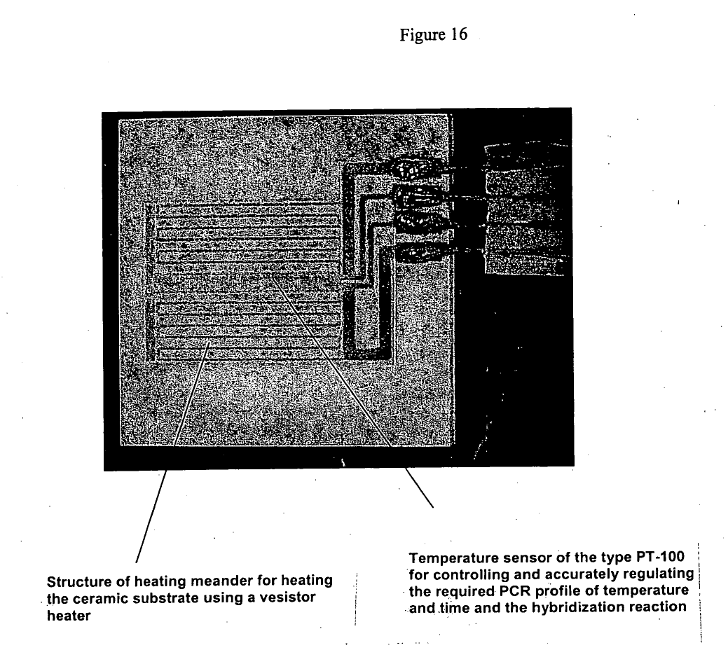

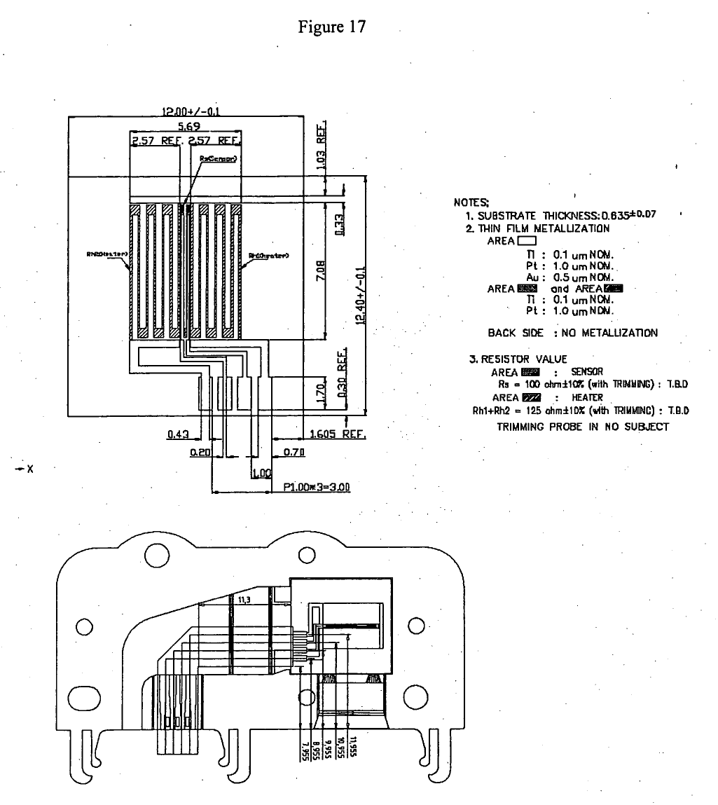

The recorded image was evaluated by means of the image evaluating software IconoClust® (Clondiag). The background-adjusted results are illustrated in FIG. 15. The specific hybridization of all six oligonucleotides used could be successfully detected.

1. A device for the amplification and detection of nucleic acids comprising:
a) a temperature controlling and/or regulating unit;
b) a reaction chamber containing a chamber support having a detection area, on which a compound library is immobilized, wherein temperature in said reaction chamber can be controlled and/or regulated by means of said temperature controlling unit and/or said regulating unit; and
c) an optical system that detects time-dependent behavior of precipitate formations on the detection area.

2. The device of claim 1, further comprising at least one fluid container which is connected with said reaction chamber.

3. The device of claim 1, further comprising a unit for controlling loading fluids and unloading fluids in said reaction chamber.

4. The device of claim 1, further comprising a unit connected with said optical system for processing signals recorded by said optical system.

5. The device of claim 1, further comprising an interface for external computers.

6. The device of claim 1, wherein said optical system further comprises a detector having a two-dimensional read out.

7. The device of claim 1, wherein said optical system further comprises a camera.

8. The device of claim 6, further comprising a light source.

9. The device of claim 1, wherein said optical system further comprises lenses, mirrors, filters, or a combination thereof.

10. The device of claim 1, wherein said optical system homogeneously illuminates said detection area.

11. The device of claim 1, wherein said optical system detects time-dependent behavior of an alteration of transmission properties of said detection area.

12. The device of claim 1, wherein said optical system detects time-dependent behavior of an alteration of reflection properties of said detection area.

13. The device of claim 8, wherein said reaction chamber and said chamber support are optically transparent in an optical path extending from said light source to said detector.

14. The device of claim 1, wherein said optical system detects time-dependent behavior of an alteration of diffusion properties of said detection area.

15. The device of claim 14, wherein said chamber support is not optically transparent in said detection area.

16. The device of claim 1, wherein said chamber support comprises a material having a thermal conductivity of 15 to 500 Wm⁻¹K⁻¹.

17. The device of claim 16, wherein said material having a thermal conductivity of 15 to 500 Wm⁻¹K⁻¹ comprises a ceramic material.

18. The device of claim 17, wherein said ceramic material is an aluminum oxide ceramic.

19. The device of claim 8, wherein said light source and said detector are arranged on the same side of said detection area.

20. The device of claim 8, wherein said detector and said light source are arranged on opposite sides of said detection area.

21. The device of claim 1, wherein said reaction chamber further comprises a data matrix containing information on a compound library and/or conduction of amplification, and/or detection reaction.

22. The device of claim 1, wherein said reaction chamber comprises a capillary gap between said chamber support and a microarray.

23. The device of claim 22, wherein said capillary gap is approximately 50 μm to 100 μm thick.

24. The device of claim 1, further comprising an electric detection system.

25. A method for detecting nucleic acids, comprising:
a) providing a device comprising:
1) a temperature controlling and/or regulating unit;
2) a reaction chamber containing a chamber support having a detection area on which a compound library is immobilized, wherein temperature in said reaction chamber can be controlled and/or regulated by means of said temperature controlling and/or regulating unit; and
3) an optical system for detecting time-dependent behavior of precipitate formations on said detection area;
b) initiating interaction of nucleic acids to be detected with a compound library immobilized on said detection area; and
c) detecting said interaction.

26. The method of claim 25, wherein said nucleic acids are labeled with a detectable marker.

27. The method of claim 25, wherein said detecting further comprises detecting interaction and precipitate as a result of a reaction leading to a precipitate on array elements.

28. The method of claim 26, wherein said detectable marker catalyzes a reaction creating conversion of a soluble compound to a precipitate at array elements, where said interaction has occurred.

29. The method of claim 25, wherein said detecting said interaction further comprises detecting single intensities of time-dependent behavior of a precipitate formation on a substrate.

30. The method of claim 28, wherein said reaction comprises a chemical reduction of a silver compound to elemental silver.

31. The method of claim 26, wherein said detectable marker further comprises gold clusters, colloidal gold particles, or a combination thereof.

32. The method of claim 28, wherein said reaction comprises a conversion of a soluble educt to a substantially insoluble product catalyzed by an enzyme.

33. The method of claim 32, wherein said reaction comprises an oxidation of 3,3′,5,5′-tetramethylbenzidine catalyzed by a peroxidase.

34. The method of claim 27, wherein said detecting is conducted by means of reflection, absorption, or diffusion of a light beam, by the precipitate.

35. The method of claim 34, wherein said light beam is selected from the group consisting of a laser beam or a light emitting diode.

36. The method of claim 27, wherein said detecting comprises measuring the alteration of electric parameters at an array element.

37. The method of claim 29, wherein said substrate is a ceramic material.

38. The method of claim 37, wherein said ceramic material is an aluminum oxide ceramic.

39. The method of claim 25, further comprising amplifying said nucleic acids to be detected before said initiating interaction of the nucleic acids to be detected with the compound library.

40. A device for the amplification and qualitative and quantitative detection of nucleic acids, comprising:
a) a temperature controlling and/or regulating unit; and
b) a reaction chamber formed between a chamber support and a microarray, wherein the microarray comprises a substrate, on which nucleic acid probes are immobilized on array elements, and temperature in said reaction chamber can be controlled and/or regulated by means of said temperature controlling and/or regulating unit; and wherein a hybridization between nucleic acids to be detected and said nucleic acid probes immobilized on said substrate is detectable by means of said device without removing molecules, which are not hybridized with nucleic acids immobilized on said substrate, from said reaction chamber.

41. The device of claim 40, wherein said reaction chamber comprises a capillary gap between said chamber support and said microarray.

42. The device of claim 41, wherein said capillary gap is approximately 50 μm to 100 μm thick.

43. The device of claim 40, further comprising a detection system.

44. The device of claim 43, wherein said detection system is an optical system.

45. The device of claim 44, wherein said optical system is a fluorescence optical system.

46. The device of claim 45, wherein said fluorescence optical system is a system depicting the total volume of said reaction chamber.

47. The device of claim 40, wherein said chamber support or said substrate comprises a material having a thermal conductivity of 15 to 500 Wm⁻¹K⁻¹.

48. The device of claim 47, wherein said substrate comprises a ceramic material.

49. The device of claim 48, wherein said ceramic material is an aluminum oxide ceramic.

50. The device of claim 40, further comprising at least one fluid container connected with said reaction chamber, or a unit for controlling loading and unloading of said reaction chamber with fluids, or a combination thereof.

51. The device of claim 43, further comprising a unit connected with said detection system for processing signals recorded by said detection system.

52. The device of claim 40, further comprising an interface for external computers.

53. The device of claim 40, wherein said reaction chamber further comprises a data matrix containing information on a compound library or conduction of amplification or detection reaction, or a combination thereof.

54. A method for the amplification and qualitative and quantitative detection of nucleic acids in a sample, comprising:
a) placing a sample having nucleic acids to be detected into a reaction chamber formed between a chamber support and a microarray, wherein said microarray comprises a substrate on which nucleic acid probes are immobilized on array elements;
b) amplifying said nucleic acids to be detected in said reaction chamber via a cyclic amplification reaction; and
c) detecting hybridization between said nucleic acids to be detected and said nucleic acid probes immobilized on said substrate, without removing molecules which are not hybridized with said nucleic acid probes immobilized on said substrate from said reaction chamber.

55. The method of claim 54, wherein said reaction chamber is a capillary gap between said chamber support and said microarray.

56. The method of claim 55, wherein said capillary gap is approximately 50 μm to 100 μm thick.

57. The method of claim 54, wherein said detecting is conducted during a cyclic amplification reaction and/or after completion of a cyclic amplification reaction.

58. The method of claim 54, wherein said nucleic acids to be detected are labeled with a detectable marker.

59. The method of claim 58, wherein said detectable marker is a fluorescence marker.

60. The method of claim 59, wherein said fluorescence marker is detected by a fluorescence optical system depicting the total volume of said reaction chamber.

61. The method of claim 54, further comprising detecting an initial concentration of nucleic acid in said sample by correlation with the number of amplification cycles.

62. The method of claim 54, wherein said sample contains a nucleic acid which hybridizes with a nucleic acid probe of said microarray in a known concentration.

63. The method of claim 59, wherein said detectable marker catalyzes a reaction creating conversion of a soluble compound to a precipitate at array elements where said interaction occurred.

64. The method of claim 54, wherein said detecting further comprises detecting single intensities of time dependent behavior of precipitate formation on said substrate.

65. The method of claim 63, wherein said reaction comprises a chemical reduction of a silver compound to elemental silver.

66. The method of claim 58, wherein said detectable marker comprises gold clusters, or colloidal gold particles, or a combination thereof.

67. The method of claim 63, wherein said reaction comprises a conversion of a soluble educt to a substantially insoluble product catalyzed by an enzyme.

68. The method of claim 67, wherein said reaction comprises an oxidation of 3,3′,5,5′-tetramethylbenzidine catalyzed by a peroxidase.

69. The method of claim 54, wherein said detecting is conducted via reflection, absorption, or diffusion of a light beam by the precipitate.

70. The method of claim 69, wherein said light beam is selected from the group consisting of a laser beam and a light emitting diode.

71. The method of claim 54, wherein said detecting further comprises measuring the alteration of electric parameters at an array element.

72. The method of claim 71, wherein said electric parameters are selected from the group consisting of conductivity, resistance, and permeability.

73. The method of claim 54, wherein said amplification is conducted using PCR.

74. The method of claim 54, wherein said substrate comprises a ceramic material.

75. The method of claim 74, wherein said ceramic material is an aluminum oxide ceramic.

76. A microarray comprising a substrate, comprising a ceramic material, on which molecular probes are immobilized on predetermined regions.

77. The microarray of claim 76, wherein said ceramic material is an aluminum oxide ceramic.

78. The microarray of claim 76, wherein said ceramic material comprises at least 99.5% aluminum oxide ceramic.

79. The microarray of claim 76, wherein said substrate further comprises a surface roughness of approximately 0.04 μm to 0.12 μm.

80. The microarray of claim 76, wherein said substrate is optically transparent.

81. The microarray of claim 76, wherein said molecular probes are immobilized on said substrate via a polymeric linker.

82. The microarray of claim 81, wherein said polymeric linker is a modified silane layer.

83. The microarray of claim 76, wherein said molecular probes are selected from the group consisting of antibodies, protein receptors, peptides and nucleic acids.

84. A method for producing a microarray, comprising immobilization of molecular probes on predetermined regions of a surface of a substrate comprising a ceramic material.

85. The method of claim 84, wherein said ceramic material comprises an aluminum oxide ceramic.

86. The method of claim 84, wherein said surface of said substrate is coated with a polymeric linker, wherein said polymeric linker immobilizes said molecular probes on said substrate surface.

87. The method of claim 86, wherein said polymeric linker is a modified silane layer.

88. A method for qualitative detection and/or quantitative detection of target molecules in a sample, comprising:
a) contacting a sample comprising target molecules with molecular probes on a microarray comprising a substrate, wherein said substrate comprises a ceramic material on which said molecular probes are immobilized on predetermined regions; and
b) detecting molecular interactions between said target molecules and said molecular probes on said microarray.

89. The method of claim 88, wherein said detecting is indicative of the genotypic state of cells.

90. The method of claim 88, wherein said detecting is indicative of the physiological state of cells.

91. The method of claim 88, wherein said detecting further comprises detecting nucleic acids or amplification and qualitative and quantitative detection of nucleic acids in a sample, or a combination thereof.
